SEQUENCE LISTING (107 sequences)

SEQ ID NO: 1 - protein, 19 aa, Staphylococcus cohnii
IVMPESSGNPNAVNPAGYR

SEQ ID NO: 2 - protein, 19 aa, Staphylococcus epidermidis
IIARESNGQLHARNASGAA

SEQ ID NO: 3 - protein, 19 aa, Staphylococcus epidermidis
NRVYNGPAASPVSEGMPIN

SEQ ID NO: 4 - protein, 19 aa, Staphylococcus warneri
IVMPESGGNPNAVNPAGYR

SEQ ID NO: 5 - protein, 19 aa, Staphylococcus pettenkoferi
IIMPESGGNPNIVNPYGYS

SEQ ID NO: 6 - protein, 19 aa, Staphylococcus petrasii
IVMPESGGNPNAVNPYGYR

SEQ ID NO: 7 - protein, 19 aa, Staphylococcus saprophyticus
IVLPESSGNPNAVNPAGYR

SEQ ID NO: 8 - protein, 19 aa, Staphylococcus gallinarum
IVLPESSGNPNAVNELGYR

SEQ ID NO: 9 - protein, 19 aa, Staphylococcus aureus
IVMPESGGNPNAVNELGYR

SEQ ID NO: 10 - protein, 19 aa, Staphylococcus schleiferi
IVMPESSGNPNAVNELGYR

SEQ ID NO: 11 - protein, 19 aa, Staphylococcus pettenkoferi
IVMPESSGNPDAVNELGYR

SEQ ID NO: 12 - protein, 19 aa, Staphylococcus nepalensis
IAQRESGGDLKAVNPSSGA

SEQ ID NO: 13 - protein, 19 aa, Staphylococcus equorum
IAERESGGDLKAVNPSSGA

SEQ ID NO: 14 - protein, 32 aa, Staphylococcus nepalensis
AMWNSIVMPESSGNPNAVNPAGYRGLGQTKES

SEQ ID NO: 15 - protein, 32 aa, Escherichia coli
QLITAIIAIESGGNPNAVSKANAIGLMQLKAS

SEQ ID NO: 16 - protein, 87 aa, Staphylococcus warneri
SSASTGGSTKAQFLANGGTEEAWNAIVMPESGGNPNAVNPAGYRGLGQTMESWGTGSVASQTKGMLNYANSRYGSLSNAIAFRQSHG

SEQ ID NO: 17 - protein, 87 aa, Streptococcus Pneumoniae; identical to SEQ ID NO: 16

SEQ ID NO: 18 - protein, 87 aa, Streptococcus pneumoniae; identical to SEQ ID NO: 16

SEQ ID NO: 19 - protein, 87 aa, Staphylococcus pasteuri
SSASTGGSTKAQFLANGGTEAAWNAIVMPESGGNPNAVNPAGYRGLGQTKESWGSGSVASQTKGMLNYANSRYGSLSSAIAFRQSHG

SEQ ID NO: 20 - protein, 87 aa, Staphylococcus pettenkoferi
SASSAGGSVKAQFLANGGTEAAWNSIIMPESGGNPNIVNPYGYSGLGQTKEAWGKGSVAQQTKGFLNYVNQRYGSIDNAINFRSTHG

SEQ ID NO: 21 - protein, 87 aa, Staphylococcus argensis
SASTSGGSVKAQFLANGGTEAAWNSIIMPESGGNPNIVNPYGYSGLGQTKEAWGKGSVAQQTKGFLNYVNQRYGSIDNAINFRNNNG

SEQ ID NO: 22 - protein, 87 aa, Staphylococcus auricularis
APASTGGSVKSQFLAAGGNEAMWNAIVLPESGGNPNAVNPAGYRGLGQTMESWGTGSVANQTKGMLNYAQQRYGSVDAAIAFRANHG

SEQ ID NO: 23 - protein, 87 aa, Staphylococcus petrasii
QSASTGGSVKSQFLANGGTEAAWNAIVMPESGGNPNAVNPYGYRGLGQTMESWGTGSVAQQTKGMINYANSRYGSMDNAIAFRASHG

SEQ ID NO: 24 - protein, 87 aa, Staphylococcus carnosus
AASSTGGSVKSQFLANGGTEAAWNAIVMPESGGNPNAVNPYGYRGLGQTMESWGTGSVAQQTKGMINYANNRYGSLDNAIAFRASHG

SEQ ID NO: 25 - protein, 87 aa, Staphylococcus devriesei
SSASTGGSVKAQFLANGGTEAAWNAIVMPESGGNPNAVNPLGYRGLGQTKESWGSGSVASQTKGLINYANSRYGSLSNAIAFRSSHN

SEQ ID NO: 26 - protein, 87 aa, Staphylococcus haemolyticus
SSASTGGSVKAQFLANGGTEAAWNAIVMPESGGNPNAVNPAGYRGLGQTMESWGTGSVASQTKGMINYANSRYGSLDAAIAFRANNG

SEQ ID NO: 27 - protein, 87 aa, Staphylococcus hominis
STASTGGSVKAQFLANGGTEAAWNAIVMPESGGNPNAVNPIGYRGLGQTKEAWGTGSVATQTQGMVNYAKSRYGSWDAAIAFRQGHG

SEQ ID NO: 28 - protein, 87 aa, Staphylococcus saprophyticus
SAASTGGSVKAQFLAAGGSEAMWNSIVLPESSGNPNAVNPAGYRGLGQTKESWGTGSVADQTKGMLNYAEQRYGSVDAALSFRASHG

SEQ ID NO: 29 - protein, 87 aa, Staphylococcus edaphicus
SAASTGGSVKAQFLAAGGSEAMWNSIVLPESSGNPNAVNPAGYRGLGQTKESWGSGSVADQTKGMLNYAKQRYGSEEAALAFRASHG

SEQ ID NO: 30 - protein, 87 aa, Staphylococcus xylosus
SAASTGGSVKAQFLAAGGTEAMWNSIVMPESSGNPNAVNPAGYRGLGQTKESWGTGSVASQTKGMINYGESRYGSMEAAMAFRASNG

SEQ ID NO: 31 - protein, 87 aa, Staphylococcus nepalensis
SAASTGGSVKAQFLAAGGSEAMWNSIVMPESSGNPNAVNPAGYRGLGQTKESWGTGSVADQTKGMLNYAKQRYGSEEAALAFRASHG

SEQ ID NO: 32 - protein, 87 aa, Staphylococcus nepalensis; identical to SEQ ID NO: 31

SEQ ID NO: 33 - protein, 87 aa, Staphylococcus cohnii
SAASTGGSVKAQFLAAGGSEAMWNSIVMPESSGNPNAVNPAGYRGLGQTKESWGTGSVADQTKGMLNYAKQRYGSEAAALEFRKNHG

SEQ ID NO: 34 - protein, 87 aa, Staphylococcus NAM3COL9
SAASTGGSVKAQFLAAGGTEAMWNTIVLPESSGNPNAVNSLGYSGLGQTKESWGTGSVATQTKGMINYAEQRYGSVDEALSFRSQNN

SEQ ID NO: 35 - protein, 87 aa, Staphylococcus OJ82
SAASTGGSVKAQFLAAGGTEAMWNTIVLPESSGNPNAVNPLGYTGLGQTKEAWGTGSVATQTKGMINYAEQRYGSIDEALSFRSQNN

SEQ ID NO: 36 - protein, 87 aa, Staphylococcus equorum
SAAATGGSVKAQFLAAGGTEAMWNTIVLPESSGNPNAVNPAGYSGLGQTKEAWGTGSVSTQTKGMLNYAKERYGSVDNAIAKRQQQG

SEQ ID NO: 37 - protein, 87 aa, Staphylococcus gallinarum
QAASTGGSVKAQFLAAGGTEAMWNTIVLPESSGNPNAVNELGYRGLGQTKESWGTGSVATQTQGMINYAKERYGSIEAALAFRSANN

SEQ ID NO: 38 - protein, 87 aa, Staphylococcus succinus
TAASTGGSVKSQFLAAGGTEAMWNTIVMPESSGNPNAVNELGYQGLGQTKESWGTGSVATQTKGMLNYAKERYGSVDAALAFRAQNN

SEQ ID NO: 39 - protein, 87 aa, Staphylococcus arlettae
SSASTGGSTKAQFLAAGGTEAMWNTIVMPESSGNPNAVNELGYRGLGQTKESWGTGSVAEQTKGMINYANERYGSVEAALDFRSSNN

SEQ ID NO: 40 - protein, 87 aa, Staphylococcus kloosii
SSASTGGSVKAQFLAAGGTEAIWNTIVLPESSGNPNAVNELGYRGLGQTKESWGTGSVAEQTKGLLNYAKQRYGSVDAAVAFRNSNN

SEQ ID NO: 41 - protein, 90 aa, Artificial Sequence
  Exemplary consensus sequence.
  MISC_FEATURE (1)..(7): Xaa is any amino acid.
  MISC_FEATURE (10)..(10): Xaa is any amino acid.
  MISC_FEATURE (12)..(12): Xaa is any amino acid.
  MISC_FEATURE (14)..(16): Xaa is any amino acid.
  MISC_FEATURE (20)..(20): Xaa is any amino acid.
  MISC_FEATURE (22)..(22): Xaa is any amino acid.
  MISC_FEATURE (24)..(25): Xaa is any amino acid.
  MISC_FEATURE (28)..(28): Xaa is any amino acid.
  MISC_FEATURE (41)..(42): Xaa is any amino acid.
  MISC_FEATURE (45)..(45): Xaa is any amino acid.
  MISC_FEATURE (54)..(54): Xaa is any amino acid.
  MISC_FEATURE (58)..(58): Xaa is any amino acid.
  MISC_FEATURE (62)..(63): Xaa is any amino acid.
  MISC_FEATURE (69)..(69): Xaa is any amino acid.
  MISC_FEATURE (73)..(74): Xaa is any amino acid.
  MISC_FEATURE (79)..(80): Xaa is any amino acid.
  MISC_FEATURE (83)..(84): Xaa is any amino acid.
  MISC_FEATURE (87)..(89): Xaa is any amino acid.
XXXXXXXSVXAXFXXXGGTXAXWXXIVXPESSGGPNAVNPXXGYXGLGQTKESXWGTXGSVXXQTKGMXNYAXXRYGSXXAAXXFRXXXG

SEQ ID NO: 42 - protein, 82 aa, Staphylococcus succinus
TSDVYSQFIEAGGTKSLWDNVVMPESSGNPDAVNELGYKGLGQTKEAWGTGSVEEQTKGMINYAEERYGSIDAAVDFHIANG

SEQ ID NO: 43 - protein, 87 aa, Staphylococcus edaphicus
QSTQSTSDVYSQFIEAGGTKALWDNIVMPESSGNPDAVNELGYRGLGQTKEAWGTGSVEEQTKGMIQYAEDRYGSIDAAIDFRLANG

SEQ ID NO: 44 - protein, 84 aa, Staphylococcus aureus
QSTNEVYKAFIQAGGTKAMWDNIVMPESGGNPNAVNELGYRGLGQTKEAWGTGSVETQTKGMINYAKERYGSIDKAIQFRIANG

SEQ ID NO: 45 - protein, 84 aa, Staphylococcus agnetis; identical to SEQ ID NO: 44

SEQ ID NO: 46 - protein, 87 aa, Staphylococcus gallinarum
TASQPTSEVYNEFIQAGGTKSLWDNVVIPESSGNPNAVNELGYRGLGQTKEAWGTGSVEEQTKGMINYAEERYGSIDGAVQFHIANG

SEQ ID NO: 47 - protein, 87 aa, Staphylococcus massiliensis
QGVDQTSTVHDRFIKAGGTEAMWQNIVLPESGGDPNAVSPQGYKGLGQTKEAWGTGTVEEQTKGMINYAEERYGSIDAAVQFRIANG

SEQ ID NO: 48 - protein, 84 aa, Staphylococcus chromogenes
TSTSTVYNEFINAGGTKAMWDHIVMPESGGNPDAVNPIGYRGLGQTKEAWGTGSVTEQTKGMVNYAKERYGSIDNAIQFRMANG

SEQ ID NO: 49 - protein, 85 aa, Staphylococcus simulans
QTSTQSVHDRFIAAGGTEAMWENIVMPESSGNPDAVNELGYRGLGQTKEYWGTGTVEEQTKGMIQYAEERYGSIDNAIQFRMANN

SEQ ID NO: 50 - protein, 85 aa, Staphylococcus EZ-P03
QTSTQSVHDRFIAAGGTEAMWEYIVMPESSGNPDAVNELGYRGLGQTKEYWGTGTVEEQTKGMIQYAEDRYGSIDNAIQFRMANN

SEQ ID NO: 51 - protein, 87 aa, Weissella halotolerans
ASASASGSVHDQFIAAGGTEAMWASIVMPESGGNPNAVNPAGYRGLGQTKEGWGSGSVAQQTQGMLNYATSRYGSVENALSFRAAHN

SEQ ID NO: 52 - protein, 87 aa, Staphylococcus pettenkoferi
TTQQSTDSVYDEFINNGGTKALWDNIVMPESGGDPNAVNELGYRGLGQTKESWGTGSVAEQTQGMVQYAKERYGSIDQAIEFRQSHG

SEQ ID NO: 53 - protein, 87 aa, Staphylococcus argensis
TAQQSTDSVYDEFINNGGTKALWDNVVMPESGGDPNAVNELGYRGLGQTKESWGTGSVAEQTQGMVQYAKERYGSIDQAIEFRQSHG

SEQ ID NO: 54 - protein, 87 aa, Staphylococcus schleiferi
TDSSNTSEVYKEFIAAGGTKALWDNIVMPESSGNPNAVNELGYRGLGQTKESWGTGSVTEQTKGMIKYAKERYGSIEAAIAFRQANN

SEQ ID NO: 55 - protein, 87 aa, Staphylococcus. sciuri
TDSSNKSEVYKEFIAAGGTKALWDNIVMPESSGNPNAVNELGYRGLGQTKESWGTGSVTEQTKGMINYAKERYGSIEAAIAFRQANN

SEQ ID NO: 56 - protein, 87 aa, Staphylococcus fleurettii
ADTSNTNEVYKEFIAAGGTKALWDNIVMPESSGNPNAVNELGYRGLGQTKEAWGTGSVTEQTKGMINYAKERYGSVEAAIAFRQANN

SEQ ID NO: 57 - protein, 87 aa, Staphylococcus stepanovicii
TQAASTDEVYKEFIAAGGTKELWDAIVMPESSGNPNAVNELGYRGLGQTKEAWGTGTVTEQTKGMINYAKERYGSVDAAIQFRLANN

SEQ ID NO: 58 - protein, 85 aa, Staphylococcus lentus
AASTNEVYKEFIAAGGTKALWDNIVMPESSGNPNAVNELGYRGLGQTKESWGTGSVTEQTKGMINYAKERYGSVEAAIAFRQANN

SEQ ID NO: 59 - protein, 87 aa, Staphylococcus kloosii
QASQSTESVHQQFLNAGGTEELWQKVVLPESGGNPNAVNELGYQGLGQTKESWGTGSVEEQTKGMVQYAKERYGSIDAAISFREANG

SEQ ID NO: 60 - protein, 87 aa, Staphylococcus arlettae
TTQQSTDAVYQEFIEAGGTPELWEKVVLPESGGDPNAVNELGYKGLGQTKEDWGTGSVETQTKGMINYAEERYGSIDAAIDFREANG

SEQ ID NO: 61 - protein, 84 aa, Staphylococcus epidermidis
NESTSSVYQEFIDAGGTKALWDSIVIPESGGNPNASNGQYHGLGQTNQSWGYGSVENQTKGMINYAKERYGSIDKAISFREANG

SEQ ID NO: 62 - protein, 86 aa, Staphylococcus nepalensis
TTQANNGVYSEFIAAGGTKALWDHIVMPESSGNPDAVNELGYRGLGQTKEAWGTGSVTEQTKGMIQYAEDRYGSIDAAIEFRVANG

SEQ ID NO: 63 - protein, 86 aa, Staphylococcus nepalensis
TTQVNNDVYSEFIAAGGTKALWDHIVMPESSGNPDAVNELGYRGLGQTKEAWGTGSVTEQTKGMIQYAEDRYGSIDAAIEFRVANG

SEQ ID NO: 64 - protein, 86 aa, Staphylococcus cohnii
TTQANNDVYSEFIAAGGTKALWDNIVMPESSGNPDAVNELGYRGLGQTKEAWGTGSVTEQTKGMIQYAEDRYGSIDAAIEFRIANG

SEQ ID NO: 65 - protein, 85 aa, Staphylococcus NAM3COL9
TQTNNDVYSEFIEAGGTKALWDNIVMPESGGDPEAVNELGYKGLGQTKEAWGSGSVEEQTKGMINYAEDRYGSIDAAIDFRLANG

SEQ ID NO: 66 - protein, 85 aa, Staphylococcus equorum
TQANNDVYSEFIEAGGTKALWDNIVMPESSGDPEAVNELGYKGLGQTKEAWGTGTVEEQTKGMINYAEDRYGSIEAAIDFRLANG

SEQ ID NO: 67 - protein, 86 aa, Staphylococcus xylosus
TTQSTNDVYDQFIEAGGTKALWDNIVMPESSGNPDAVNELGYRGLGQTKESWGTGSVEEQTKGMINYAEERYGSVDAAIDFRVANG

SEQ ID NO: 68 - protein, 85 aa, Staphylococcus saprophyticus
TQSNNDVYSQFIEAGGTKALWDNIVMPESSGNPDAVNELGYRGLGQTKEAWGTGSVEEQTKGMIKYAEERYGSIDAAIDFRLANG

SEQ ID NO: 69 - protein, 88 aa, Staphylococcus nepalensis
ASTQSSSSNEASSGSSVNVNSHLQAIAQRESGGDLKAVNPSSGAAGKYQFLQSTWDSVAPAEYQGVSPTEAPESVQDQAAMDLYNTAG

SEQ ID NO: 70 - protein, 88 aa, Staphylococcus nepalensis
ASTQSSSSNEASSGSSVNVNSHLQAIAQRESGGDLKAVNPSSGAAGKYQFLQSTWDSVAPAEYQGVSPTEAPESVQDAAAVKLYNEVG

SEQ ID NO: 71 - protein, 88 aa, Staphylococcus cohnii
ALTQSSNSNEASSGSSVNVNSHLQAIAQRESGGDLKAVNPSSGAAGKYQFLQSTWDSVAPAQYKGVSPTEAPESVQDAAAVKLYNEVG

SEQ ID NO: 72 - protein, 88 aa, Staphylococcus saprophyticus
STSNESSSSEASEGSSVNVNSHLQAIAQRESGGDLKAVNPSSGAAGKYQFLQSTWDSVAPSEYQGVSPTEAPEAVQDAAAVKLYNTAG

SEQ ID NO: 73 - protein, 88 aa, Staphylococcus xylosus
TSSNESSSSEASEGSSVNVNSHLQAIAQRESGGDLKAVNPSSGAAGKYQFLQSTWDSVAPSQYKGVSPTEAPESVQDAAAVKLYNEVG

SEQ ID NO: 74 - protein, 88 aa, Staphylococcus HMSC034G07
SQQSTGSSSEASSGSSVDVNDHLKQIAQRESGGDLKAVNPSSGAAGKYQFLQSTWDSVAPDEYKGVSPTEAPESVQDQAAMDLYESEG

SEQ ID NO: 75 - protein, 88 aa, Staphylococcus equorum
ESSNESSSSEASSGSSVEVNDHLKQIAERESGGDLKAVNPSSGAAGKYQFLQSTWDSVAPAEYQGQSPTEAPESVQDQAAMDLYESEG

SEQ ID NO: 76 - protein, 86 aa, Staphylococcus succinus
ATTTQSSQASEGSSVNVNAHLQQIAQRESGGDLKAVNPSSGAAGKYQFLQSTWDSVAPAEYKGVSPTEAPESVQDQAAVDLYNSAG

SEQ ID NO: 77 - protein, 88 aa, Staphylococcus gallinarum
QQTSNSSSSEESTGSSVNVNSHLQQIAQRESGGDLKAVNPSSGAAGKYQFLQSTWDSVAPDEYKGVSPTEAPESVQDAAAVKLYNTAG

SEQ ID NO: 78 - protein, 88 aa, Staphylococcus ZWU0021
SSSNESSSSEASESSSSGVNAHLQQIAQRESGGDIHATNPSSGASGKFQFLQSTWDSVAPAEYQGQPAASAPESVQDAAAQKLYDTEG

SEQ ID NO: 79 - protein, 88 aa, Staphylococcus nepalensis
STQSSDSSSEASSGSSVEVNDHLKQIAERESGGDLKAVNPSSGAAGKYQFLQSTWDSVAPAEYQGQSPTEAPESVQDQAAMDLYESEG

SEQ ID NO: 80 - protein, 88 aa, Staphylococcus nepalensis; identical to SEQ ID NO: 79

SEQ ID NO: 81 - protein, 88 aa, Staphylococcus NAM3COL9
DSSNESSSSEASSGSSVQVNDHLKGIAERESGGDLKAVNASSGAAGKYQFLQSTWDSVAPEQYQGQSPTEAPESVQDQAAMDLYESEG

SEQ ID NO: 82 - protein, 88 aa, Staphylococcus edaphicus
SSSNESSSSKASEGSSVNVNSHLQAIAQRESGGDLKAVNPSSGAAGKYQFLQSTWDSVAPAEYQGVSPTEAPEAVQDAAAVKLYNTAG

SEQ ID NO: 83 - protein, 88 aa, Staphylococcus saprophyticus; identical to SEQ ID NO: 78

SEQ ID NO: 84 - protein, 88 aa, Staphylococcus xylosus; identical to SEQ ID NO: 73

SEQ ID NO: 85 - protein, 88 aa, Staphylococcus cohnii
QQSTDSSSSEASSGSSVDVNDHLKQIAQRESGGDLKAVNPSSGAAGKYQFLQSTWDSVAPAEYKGVSPTEAPESVQDQAAMDLYESAG

SEQ ID NO: 86 - protein, 88 aa, Staphylococcus nepalensis
STQSSDSSSEASSGSSVDVNDHLKQIAERESGGDLKAVNPSSGAAGKYQFLQSTWDSVAPAEYQGQSPTEAPESVQDQAAMDLYESEG

SEQ ID NO: 87 - protein, 88 aa, Staphylococcus nepalensis; identical to SEQ ID NO: 86

SEQ ID NO: 88 - protein, 86 aa, Staphylococcus succinus; identical to SEQ ID NO: 76

SEQ ID NO: 89 - protein, 88 aa, Staphylococcus nepalensis; identical to SEQ ID NO: 69

SEQ ID NO: 90 - protein, 88 aa, Staphylococcus nepalensis; identical to SEQ ID NO: 70

SEQ ID NO: 91 - protein, 88 aa, Staphylococcus cohnii
ASTQSSSSNEASSGSSVNVNSHLQAIAQRESGGDLKAVNPSSGAAGKYQFLQSTWDSVAPAEYKGVSPTEAPESVQDAAAVKLYNEVG

SEQ ID NO: 92 - protein, 86 aa, Staphylococcus succinus; identical to SEQ ID NO: 76

SEQ ID NO: 93 - protein, 75 aa, Staphylococcus cohnii
TSSIDAIANQMASRTGVSAAQWKGVIQRESGGNANAVNASSGAYGLFQLLGHGEHSGMSVQDQIDTAVGVYKSQG

SEQ ID NO: 94 - protein, 74 aa, Staphylococcus aureus
AGATGSSAAQIMAQRTGVSASTWAAIIARESNGQVNAYNPSGASGLFQTMPGWGPTNTVDQQINAAVKAYKAQG

SEQ ID NO: 95 - protein, 63 aa, Streptococcus pneumoniae
AGSTGSYAAQEMAKRTGVSASTWEYIIARESNGQANARNASGASGLFQTMPGWGSTASVSDQI

SEQ ID NO: 96 - protein, 19 aa, Staphylococcus Nepalensis
IVMPESSGNPNAVNPAGYR

SEQ ID NO: 97 - protein, 19 aa, Staphylococcus cohnii
VIQRESGGNANAVNASSGA

SEQ ID NO: 98 - protein, 247 aa, Streptococcus pneumoniae
MMKKTFIASTLALTLGATGYAVSGHEAHASETTNVDQAHLVDLAHNHPEQLNAAPVQEGAYDIHFVSGGFEYNFTSDGTNFSWNYQEAGSTSAQTSNTAVQSADYTTSYNQEAGTQSVSSNQQSSNTNVEAVSAPTTSNNGSNHNYSTKTTSYSAPSTSSASTGGSTKAQFLANGGTEEAWNAIVMPESGGNPNAVNPAGYRGLGQTMESWGTGSVASQTKGMLNYANSRYGSLSNAIAFRQSHGWW

SEQ ID NO: 99 - protein, 246 aa, Streptococcus pneumoniae
MKKTFIASTLALTLGAAGYAVSGHEAHASETTNVDQAHLVDLAHNHPEQLNAAPVQEGAYDIHFVSGGFEYNFTSDGTNFSWNYQEVGSTSAQTSNTAVQSADYTTSYNQEAGTQSVSSNQQSSNTNVEAVSAPTTSNNGSNHNYSTKTTSYSAPSTSSASTGGSTKAQFLANGGTEEAWNAIVMPESGGNPNAVNPAGYRGLGQTMESWGTGSVASQTKGMLNYANSRYGSLSNAIAFRQSHGWW

SEQ ID NO: 100 - protein, 246 aa, Staphylococcus warneri
MKKTFIVSTLALTLGAAGYAVSGHEAHASETTNVDQAHLVDLAHNHPEQLNAAPVQEGAYDIHFVSGGFEYNFTSDGTNFSWNYQEAGSTSAQTSNTAVQSADYTTSYNQEAGTQSVSSNQQSSNTNVEAVSAPTTSNNGSNHNYSTKTTSYSAPSTSSASTGGSTKAQFLANGGTEEAWNAIVMPESGGNPNAVNPAGYRGLGQTMESWGTGSVASQTKGMLNYANSRYGSLSNAIAFRQSHGWW

SEQ ID NO: 101 - DNA, 742 bp, Streptococcus pneumoniae
cctaccaccaaccgtggctttgacggaaagcaattgcattgcttaatgaaccgtaacggctattagcatagttaagcatacctttagtttggctagctactgaaccagttccccatgattccatagtttgtcctaaacctctgtaaccagctgggtttactgcgttagggttaccacctgattctggcataacgatagcgttccaagcttcttcagtaccaccattagctaagaattgtgctttagttgatccacctgttgaagcacttgaagttgatggtgctgagtatgaagttgttttagtgctgtagttgtggtttgaaccattgtttgatgtagttggagctgaaacagcttctacattagtgttgcttgattgttggttagagcttactgattgagtaccagcttcttgattgtaagaagttgtgtagtcagctgattgaacagcagtgtttgatgtttgagctgaagtagaaccaacttcttggtagttccaagagaagttagtaccatctgaagtgaagttatattcgaatccaccacttacaaagtgaatgtcataagcgccttcttgaactggtgcagcgtttaattgttctgggtggttatgagctaagtctactaagtgtgcttgatctacgttagtagtttctgaagcgtgtgcttcgtgtcctgatactgcgtaacctgctgcgcctaatgttaatgctaaagttgatgcgataaatgtcttcttcat

SEQ ID NO: 102 - DNA, 742 bp, Staphylococcus warneri
cctaccaccaaccgtggctttgacggaaagcaattgcattgcttaatgaaccgtaacggctattagcatagttaagcatacctttagtttggctagctactgaaccagttccccatgattccatagtttgtcctaaacctctgtaaccagctgggtttactgcgttagggttaccacctgattctggcataacgatagcgttccaagcttcttcagtaccaccattagctaagaattgtgctttagttgatccacctgttgaagcacttgaagttgatggtgctgagtatgaagttgttttagtgctgtagttgtggtttgaaccattgtttgatgtagttggagctgaaacagcttctacattagtgttgcttgattgttggttagagcttactgattgagtaccagcttcttgattgtaagaagttgtgtagtcagctgattgaacagcagtgtttgatgtttgagctgaagtagaaccagcttcttggtagttccaagagaagttagtaccatctgaagtaaagttatattcgaatccaccacttacaaagtgaatgtcataagcgccttcttgaactggtgcagcgtttaattgttctgggtggttatgagctaagtctactaagtgtgcttgatctacgttagtagtttctgaagcgtgtgcttcgtgtcctgatactgcgtaacctgctgcgcctaatgttaatgctaaagttgatgcgataaatgtcttcttcat

SEQ ID NO: 103 - protein, 19 aa, Streptococcus pneumoniae
IVMPESGGNPNAVNPAGYR

SEQ ID NO: 104 - protein, 241 aa, Mycobacterium abscessus
MKKTVIASSLAVTLGLTGYALTNDHSAHASEQTTNYSHLADLAQNNPSELNAHPVQAGAYDISFVKDGFKYNFTSNGNTWSWNYTYTGGADTAQSTTDYTESYNQASTQSVSSNNQASTSNVKAVSAPVQRTSSYNNYSARTTSYSAPKTTSYSTASTGGSVKAQFLANGGTEAAWNAIVMPESSGNPNASNGQYHGLGQTNQSWGTGSVASQTQGMANYAKSRYGSWDAAIAFRNANGWW

SEQ ID NO: 105 - protein, 241 aa, Staphylcoccus hominis
MKKTVIASSLAVTLGLTGYALTNDHSAHASEQTTNYSHLADLAQNNPSELNAHPVQAGAYNISFVKDGFKYNFTSNGNTWSWNYTYTGGADTAQSTTDYTESYNQASTQSVSSNNQASTSNVKAVSAPVQRTSSYNNYSVRTTSYSAPKTTSYSTASTGGSVKAQFLANGGTEAAWNAIVMPESSGNPNASNGQYHGLGQTNQSWGTGSVASQTQGMANYAKSRYGSWDAAIAFRNANGWW

SEQ ID NO: 106 - protein, 18 aa, Mycobacterium abscessus
IVMPESSGNPNASNGQYH

SEQ ID NO: 107 - protein, 18 aa, Staphylococcus hominis
IVMPESSGNPNASNGQYH

(19) United States
(12) Patent Application Publication
(10) Pub. No.: US 2023/0357373 A1
(43) Pub. Date: Nov. 9, 2023

(54) METHODS AND COMPOSITIONS FOR EVALUATING AND TREATING FIBROSIS

(71) Applicants: Mie University, Tsu-shi (JP); The Board of Trustees of the University of Illinois, Urbana, IL (US)

(72) Inventors: Esteban Gabazza, Tsu-shi (JP); Corina D'Alessandro-Gabazza, Tsu-shi (JP); Isaac Cann, Urbana, IL (US)

(73) Assignees: Mie University, Tsu-shi (JP); The Board of Trustees of the University of Illinois, Urbana, IL (US)

(21) Appl. No.: 17/786,449

(22) PCT Filed: Dec. 16, 2020
(86) PCT No.: PCT/US2020/065280

§ 371 Date: Jun. 16, 2022

Related U.S. Application Data
(60) Provisional application No. 62/948,983, filed on Dec. 17, 2019.

(51) Int. Cl. C07K 16/12; G01N 33/68; A61P 11/00

(52) U.S. Cl. CPC C07K 16/1271; G01N 33/6893; A61P 11/00; A61K 2039/505

(57) ABSTRACT

Staphylococcus nepalensis releases corisin, a peptide conserved in diverse Staphylococci, that induces apoptosis of lung epithelial cells. Therefore, methods and apparatus for detecting the presence of corisin in a biological sample of a patient are disclosed, as well as pharmaceutical compositions, such as antibodies, and methods for treating patents having or suspected of having fibrosis.

### CROSS-REFERENCE

This application claims priority to U.S. Patent Application No. 62/948,983 filed on Dec. 17, 2019, the contents of which are fully incorporated herein.

### TECHNICAL FIELD

The present invention generally relates to a Staphylococcus pro-apoptotic peptide (herein called “corisin”) that has been found to induce acute exacerbation of pulmonary fibrosis, as well as to methods, kits and apparatus for diagnosing or evaluating fibrosis in patients and to methods and compositions for ameliorating or treating fibrosis, such as idiopathic pulmonary fibrosis.

### BACKGROUND ART

Idiopathic pulmonary fibrosis (IPF) is a chronic and fatal disease of as yet undetermined etiology; however, apoptosis of lung alveolar epithelial cells is known to play a role in disease progression. This intractable disease is associated with increased abundance of Staphylococcus and Streptococcus in the lungs, yet their roles in disease pathogenesis have remained elusive.

IPF is the most frequent form of idiopathic interstitial pneumonitis characterized by a chronic, progressive and fatal clinical outcome. See NPL1 and NPL2 (the full citations for all Non-Patent Literature Documents identified herein by the designation “NPL” are provided at the end of the present specification). The prognosis of IPF is worse than in many other types of malignancy, with a life expectancy for patients following diagnosis of the disease being only 2 to 3 years. See NPL3 and NPL4. Repetitive injury and/or apoptosis of lung epithelial cells, excessive release of profibrotic factors and enhanced lung recruitment of extracellular matrix-producing myofibroblasts play critical roles in the disease pathogenesis. See NPL2 and NPLS.

NPL6 suggests that the lung microbiome plays a causative role in IPF, with increased lung bacterial burden being associated with acute exacerbation of the disease and high mortality rate. As shown in NPL7, the relative abundance of lung microbes of the Staphylococcus and Streptococcus genera has also been associated with acceleration of the clinical progression of IPF. However, the role of these bacteria in the pathogenesis of pulmonary fibrosis has remained unclear. The capacity to culture the bacteria associated with fibrotic tissues and elucidation of their phenotypic characteristics would be ideal in clearly identifying the organisms involved in the pathogenesis of IPF; however, it is believed there has been no earlier report of bacterial isolates that are relevant to disease pathogenesis.

In NPL8 and NPL9, it was demonstrated that the lung fibrotic tissue from IPF patients and from transforming growth factor (TGF)β1 transgenic (TG) mice with lung fibrosis is characterized by an enrichment of halophilic bacteria. NPL4 substantiated this observation.

### SUMMARY OF THE INVENTION

The results in NPL8 and NPL9 led us to hypothesize that the fibrotic tissue is a salty microenvironment, and that the hypersaline condition of the lung fibrotic tissue facilitates the growth of bacteria that release factors that play a role in IPF disease pathogenesis and its acute exacerbation.

In our research that led to the developments and insights described herein, we used a halophilic medium to enrich for Staphylococcus strains from lung fibrotic tissue samples originating from TGFβ1 TG mice. As a result, we found that the culture supernatants of one of the bacterial strains, namely S. nepalensis strain CNDG, contain a pro-apoptotic peptide that induces apoptosis of lung epithelial cells.

We further found that this pro-apoptotic peptide, designated herein as “corisin”, is a component of a transglycosylase conserved in diverse members of the genus Stapylococcus, and that intratrachael instillation of mice having established lung fibrosis either with corisin or the corisin-encoding S. nepalensis strain CNDG leads to acute exacerbation of the disease.

Furthermore, by performing enhanced detection of corisin in human IPF patients with acute exacerbation and comparing these results to patients without disease exacerbation, we concluded that bacteria carrying and shedding the pro-apoptotic peptide are involved in acute exacerbation of pulmonary fibrosis.

More specifically, we have found that Staphylococcus nepalensis releases corisin, a peptide conserved in diverse Staphylococci, to induce apoptosis of lung epithelial cells. The disease in mice exhibits acute exacerbation after intrapulmonary instillation of corisin or after lung infection with corisin-harboring S. nepalensis compared to untreated mice or mice infected with bacteria lacking corisin. Correspondingly, the lung corisin levels are significantly increased in human IPF patients with acute exacerbation compared to patients without disease exacerbation. This resulted in the conclusion that bacteria, which shed corisin, are involved in acute exacerbation of IPF, yielding insights to the molecular basis for the elevation of Staphylococci in pulmonary fibrosis and for the association of the Staphylococci with the worsening stage of pulmonary fibrosis.

Based on these developments and insights, we developed the following aspects of the present teachings.

In one aspect of the present teaching, methods, kits and apparatus are disclosed that comprise detecting the presence of corisin in a biological sample of the patient, preferably detection that is performed in vitro. The corisin may have, e.g., one of the amino acid sequences of SEQ ID NO: 1, SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, or SEQ ID No: 13 disclosed herein. These methods, kits and/or apparatus may be used in the evaluation and/or diagnosis of fibrosis in the patient, such as idiopathic pulmonary fibrosis (IPF), liver cirrhosis, kidney fibrosis, cystic fibrosis, myelofibrosis, and/or mammary fibrosis. Preferably, these methods, kits and/or apparatus is (are) used in the detection and/or evaluation of idiopathic pulmonary fibrosis (IPF).

In such a method, kit or apparatus, the corisin may be detected by mass spectrometry, Western blotting, and/or enzyme-linked immunosorbent assay (ELISA) and may involve binding of the corisin to an antibody, preferably in vitro. For example, the antibody may recognize (bind to), e.g., one of the amino acid sequences of SEQ ID NO: 1, SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, or SEQ ID No: 13 disclosed herein.

In another aspect of the present teachings, an antibody that binds to corisin is disclosed. The antibody may recognize (bind to) one of the amino acid sequences of SEQ ID NO: 1SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, or SEQ ID No: 13 disclosed herein and it may be a polyclonal antibody.

The antibody may be used as a medicament in preventing, ameliorating and/or treating fibrosis in a patient subject having, or suspected of having or developing, fibrosis. For example, the antibody may be provided in a pharmaceutical composition for use as a medicament to be administered to a patient in need thereof.

Such pharmaceutical compositions optionally may include one or more pharmaceutically acceptable additives, salts and/or excipients, such as preservatives, saccharides, solubilizing agents, stabilizers, carriers, diluents, bulking agents, pH buffering agents, tonicifying agents, antimicrobial agents, wetting agents, and/or emulsifying agents, preferably in an amount (e.g., a combined amount, if two or more are present) of 0.005% to 99% by weight, e.g., 0.5% to 98% by weight.

The antibody may be used in preventing, ameliorating and/or treating idiopathic pulmonary fibrosis (IPF), liver cirrhosis, kidney fibrosis, cystic fibrosis, myelofibrosis, and/or mammary fibrosis. For example, the antibody may be used in preventing, ameliorating and/or treating idiopathic pulmonary fibrosis (IPF). The antibody may be a neutralizing antibody, e.g., an antibody that blocks or inhibits negative effects of corisin in the lungs or other tissue of a patient suffering from fibrosis.

In a further aspect of the present teachings, a method of treating fibrosis in a patient in need thereof may comprise administering a therapeutically effective amount of any of the above-described antibodies the patient. For example, the antibody may be administered to one or both lungs of the patient. In addition or in the alternative, the antibody may be administered intraperitoneally or by intratracheal instillation or by inhalation. Administration of the antibody preferably at least reduces the severity of the fibrosis in the subject.

It is noted that all methods of diagnosis and/or evaluation are preferably performed in vitro on a biological sample that was extracted, collected, obtained, etc. from a patient having, or suspected of having or developing, fibrosis, such as any of the types of fibrosis described above or below.

Other objects, aspects, embodiments and advantages of the present teachings will become apparent to a person skilled in the art upon reading the following detailed description in view of the Figures and appended claims.

### BRIEF DESCRIPTION OF THE FIGURES

FIG. 1A shows chest computed tomography (CT) images of nine wild-type (WT) mice, six TGFβ1 TG mice without fibrosis and six TGFβ1 TG mice with fibrosis; FIG. 1B shows CT scores for these mice; FIG. 1C shows saline contents in the lung tissue of these mice as measured by microwave analysis/inductively coupled plasma mass spectrometry.

FIGS. 2A and 2B respectively show CT images and CT fibrosis scoring of wild-type (WT) mice (n=3) and TGFβ1 transgenic (TG) mice (n=8).

FIG. 2C shows fibrotic lung tissues excised under sterile conditions from wild-type (n=3) and TGFβ1 transgenic (n=8) mice after culturing in hypersaline culture media for 48 h. Analysis of bacterial colonies was performed by transmission electron microscope. Scale bars indicate 100 nm.

FIG. 2D shows a flow cytometry analysis of A549 alveolar epithelal cells cultured for 48 h in DMEM medium containing 1/10 diluted spent culture supernatant of the mixture of Staphylococcus spp. (strain 6; n=9), Staphylococcus nepalensis strain CNDG (n=9), or control medium (n=9).

FIG. 2E shows a flow cytometry analysis of normal human bronchial epithelial cells after culturing for 48 h in DMEM medium containing 1/10diluted spent culture supernatant of the mixture of Staphylococcus spp. (strain 6; n=8), Staphylococcus nepalensis strain CNDG (n=8), or control medium (n=4).

FIGS. 2F and 2G show a TUNEL assay after culturing A549 alveolar epithelial cells in the presence of medium (n=6) or supernatant of Staphylococcus nepalensis strain CNDG (n=6). Scale bars indicate 20 μm.

FIG. 3A shows absorbance of fractions from the culture supernatant of the mixture of Staphylococcus spp. after gel filtration using Sephadex G25 column; FIG. 3B shows cell viability after treating A549 alveolar epithelial cells with the culture supernatant of the mixture of Staphylococcus spp. (each fraction n=3); FIG. 3C shows cells in sub-G1 phase after treating A549 cells with culture supernatant of the mixture of Staphylococcus spp. (each fraction n=3).

FIG. 3D shows representative histograms of A549 cells in sub-G1 phase after treatment with culture supernatant of the mixture of Staphylococcus spp.

FIG. 3E shows absorbance of fractions from the culture supernatant of Staphylococcus nepalensis strain CNDG after gel filtration; FIG. 3F shows cell viability after treating A549 cells with culture supernatant of Staphylococcus nepalensis strain CNDG (each fraction n=3); FIG. 3G shows cells in sub-G1 phase after treating A549 cells with culture supernatant of Staphylococcus nepalensis strain CNDG (each fraction n=3).

FIG. 3H shows representative histograms of A549 cells in sub-G1 phase after treatment with culture supernatant of Staphylococcus nepalensis strain CNDG. (One mL of each sample was applied into the Sephadex G25 column. The material eluted was collected in 2 ml fractions and then absorbance was measured at 280 nm. Cell viability was evaluated by using a commercial cell counting kit and the percentage of cells in sub-G1 by flow cytometry.)

FIGS. 3I, 3J and 3K show bacteria were cultured in medium containing 2% or 8% salt and then the culture supernatants of the mixture of Staphylococcus spp. (n=9), Staphylococcus nepalensis CNDG strain (n=9) or medium (n=9) were prepared by centrifugation and respectively added to a culture medium of A549 alveolar epithelial cells at 1/10 dilution. Flow cytometry of A549 cells was performed after staining with propidium iodide and annexin V.

FIGS. 4A, 4B and 4C show culture supernatant from bacteria was separated into fractions of <10 kDa and >10 kDa by filtration and each fraction was added to A549 alveolar epithelial cells after 1/10 dilution to determine apoptosis by flow cytometry.

FIGS. 5A-5C show a structural alignment analysis for corisin; FIGS. 5D and 5E show that synthetic corisin peptides exhibited a pro-apoptotic effect of the staphylococcal isolate supernatant in a dose dependent manner as a result of a flow cytometry analysis of A549 alveolar epithelial cells performed after culturing for 48 h in DMEM medium containing increasing concentrations of the pro-apoptotic peptide; FIG. 5F shows electron micrographs of A549 alveolar epithelial cells respectively treated with saline or corisin.

FIG. 6A shows a schedule for treating mice with saline, scrambled peptide or corisin.

FIG. 6B shows a counting of bronchoalveolar lavage fluid cells for three WT mice treated with saline (WT/SAL), five TGFβ1 TG mice treated with saline (TGFβ1 TG/SAL), four TGFβ1 TG mice treated with scrambled peptide (TGFβ1 TG/scrambled) and four TGFβ1 TG mice treated with corisin (TGFβ1 TG/corisin), wherein the scale bars indicate 100 μm.

FIGS. 6C and 6D show quantification of collagen area by WinROOF software wherein the scale bars indicate 100 μm.

FIG. 6E shows the concentrations of TGFβ1, monocyte chemoattractant protein (MCP)-1 and collagen I were measured by enzyme immunoassays, wherein n=3 in the WT/SAL group, n=5 in the TGFβ1 TG/SAL and TGFβ1 TG/corisin groups, and n=4 in the TGFβ1 TG/scrambled peptide group.

FIGS. 6F and 6G show DNA fragmentation as evaluated by staining through terminal deoxynucleotidyl transferase dUTP Nick-End Labeling (TUNEL), wherein the scale bars indicate 50 μm and n=3 in the WT/SAL group, n=5 in the TGFβ1 TG/SAL and TGFβ1 TG/corisin groups, and n=4 in the TGFβ1 TG/scrambled peptide group.

FIGS. 7A and 7B show the numbers of cells in bronchoalveolar lavage fluid (BALF) that were counted and then stained with Giemsa on the second day after intratracheal instillation of saline or each bacterium, wherein the scale bars indicate 100 μm.

FIGS. 7A and 7B show DNA fragmentation as evaluated by staining with terminal deoxynucleotidyl transferase dUTP Nick-End Labeling (TUNEL), and then quantifying using the image WinROOF software.

FIGS. 8A and 8B respectively show photographs of Western blotting of corisin in lung tissue from four WT mice and four TGFβ1 TG mice and the respective ratios of corisin to β-actin. Quantification was performed using ImageJ software.

FIG. 8C shows corisin levels as measured using a competitive enzyme immune assay for eight healthy controls, and thirty-four patients with stable idiopathic pulmonary fibrosis (IPF) patients.

FIG. 8D shows an analysis of bronchoalveolar lavage fluid levels of corisin in fourteen of the IPF patients before and after acute exacerbation.

FIGS. 9A and 9B show criteria for scoring lung radiological findings and correlation of CT score with the Ashcroft fibrosis score and with the hydroxyproline content of the lungs.

FIGS. 10A-10D show abnormal immune responses in lung fibrotic tissue and respectively show the percentages of monocytes/macrophages, CD4Cd25 cells, T cells and B cells in lung fibrotic tissue of mice treated in three different ways.

FIG. 11 shows that the level of sodium correlates with the number of immune cells, and with the expression of fibrotic markers and sodium channels, in lung fibrotic tissues.

FIGS. 12A-12D show that the pro-apoptotic factor in culture supernatant from bacteria is heat-stable.

FIG. 13 is a schematic diagram describing sample fractionation steps and the bioactivity of each fraction.

FIG. 14 shows the pro-apoptotic activity of each of the fractions, which were obtained by fractionation of bacterial supernatant from Staphylococcus nepalensis, on A549 alveolar epithelial cells.

FIG. 15 shows that ethanol, methanol and acetonitrile fractions of the culture supernatants of Staphylococcus nepalensis strain CNDG induced apoptosis of lung epithelial cells.

FIGS. 16A, 16B and 16C show that the pro-apoptotic activity of the fractions obtained from the supernatants of cultured Staphylococcus nepalensis strain CNDG is sensitive to proteinase K treatment.

FIG. 17 is a photograph of silver staining of the fraction that exhibited pro-apoptotic activity.

FIGS. 18A-18E show that synthetic corisin peptide prepared by a different manufacturer induced dose-dependent apoptosis of alveolar epithelial cells, and the apoptotic activity of corisin was significantly more potent than an equal concentration of supernatant protein.

FIGS. 19A-19E show that the pro-apoptotic peptide (corisin) induces apoptosis of normal human bronchial epithelial cells, but its scrambled sequence did not.

FIGS. 20A-20E show that the synthetic pro-apoptotic peptide (corisin) is heat-stable.

FIGS. 21A-21F show that the apoptotic peptide (corisin) does not induce apoptosis of fibroblast, vascular endothelial cells or T cells.

FIGS. 22A and 22B each show a band at the corresponding molecular weight of corisin as observed in Western blotting of mouse lung tissue samples and culture supernatant of Staphylococcus nepalensis using a corisin antibody.

FIGS. 23A-23D show that antibody against corisin inhibits both the pro-apoptotic activity of corisin and the pro-apoptotic activity of the supernatant of Staphylococcus nepalensis strain CNDG.

FIGS. 24A-24E show that full-length transglycosylase 351 containing the corisin sequence has no apoptotic activity.

FIGS. 25A and 25B respectively show CT images and findings in mice used for intratracheal instillation of corisin, scrambled peptide or saline.

FIGS. 26A and 26B respectively show CT images and findings in mice used for intratracheal instillation of Staphylococcus nepalensis, Staphylococcus epidermidis or saline.

FIGS. 27A and 27B show the synthetic peptide containing the sequence of the transglycosylase segment (corisin) from Staphylococcus nepalensis strain CNDG, but not its scrambled peptide or a synthetic peptide containing the sequence of the transglycosylase segment from Staphylococcus epidermidis, induces apoptosis of alveolar epithelial cells.

FIGS. 28A and 28B show deterioration of radiological findings in germ-free TGFβ1 TG mice after intratracheal instillation of Staphylococcus nepalensis.

FIGS. 29A-29D shows a phylogenetic analysis of the Staphylococcus nepalensis strain CNDG transglycosylases and their relatives in the genus Staphylococcus.

FIGS. 30A, 30B and 30C show multiple sequence alignment of a conserved sequence of the pro-apoptotic segment of transglycosylases in several species of Staphylococcus and Streptococcus. Corisins shown in FIGS. 30A to 30C include, for example, IVMPESGGNPNAVNPAGYR (SEQ ID NO:4), IIMPESGGNPNIVNPYGYS (SEQ ID NO:5), IVMPESGGNPNAVNPYGYR (SEQ ID NO:6), IVLPESSGNPNAVNPAGYR (SEQ ID NO:7), IVLPESSGNPNAVNELGYR (SEQ ID NO:8), IVMPESGGNPNAVNELGYR (SEQ ID NO.9), IVMPESSGNPNAVNELGYR (SEQ ID NO.10), IVMPESSGNPDAVNELGYR (SEQ ID NO.11), IAQRESGGDLKAVNPSSGA (SEQ ID NO. 12), and IAERESGGDLKAVNPSSGA (SEQ ID NO. 13), which may be used in one or more aspects of the present teachings.

FIGS. 31A-31F show genomic context and multiple sequence alignment for a conserved sequence of the pro-apoptotic segment of transglycosylases in several species of Staphylococcus and Streptococcus; more particularly, FIG. 31A shows the genomic context of transglycosylases containing the peptide IVMPESSGNPNAVNPAGYR (SEQ ID NO:1) or its derivative in Staphylococcus nepalensis strain SNUC 4025 and Staphylococcus cohnii subspecies cohnii.; FIG. 31B shows Streptococcus pneumoniae contains transglycosylases (COE35810 and COE67256) with an almost identical peptide sequence to corisin; FIG. 31C shows the query sequence and the subject sequence in the alignment are from S. pneumoniae strain N and S. warneri, respectively (The complementary nucleotide sequence encodes COE67256 and highly identical proteins in Staphylococcus warneri strain SWO, strain SGI, strain NCTC11044, strain NCTC7291, and strain 22.1); FIG. 31D shows the genomic context of transglycosylases containing the corisin sequence or its derivative in Streptococcus pneumoniae strain N and Staphylococcus warneri; FIG. 31E shows that the genome of a strain of the emerging pathogen Mycobacterium [Mycobacteroides] abscessus harbors a transglycosylase (SKT99287) that is almost identical to a transglycosylase (WP_049379270) in Staphylococcus hominis; FIG. 31F shows the genomic context of transglycosylases containing the corisin sequence or its derivative in Mycobacterium [Mycobacteroides] abscessus and Staphylococcus hominis.

FIGS. 32A and 32B show that the synthetic peptide from Streptococcus pneumoniae strain N transglycosylase has pro-apoptotic activity.

FIG. 33 is a model of fibrotic tissue developed based on the research disclosed in this specification, in particular based on the contribution of corisin to the pathogenesis of idiopathic pulmonary fibrosis (IPF).

FIGS. 34A-34C show flow cytometry gating strategies used in the experiments described in FIG. 12A (FIG. 34A), FIG. 19A (FIG. 34B), and FIG. 20A (FIG. 34C), wherein SSC means side scatter and FSC means forward scatter.

### DETAILED DESCRIPTION OF THE INVENTION

In another aspect of the present teachings, a method for evaluating or diagnosing a subject having, or suspected of having or developing, fibrosis, may include receiving an in vitro biological sample that was collected, harvested, obtained, etc. from the subject; and detecting an amount of corisin that is present in the biological sample. Such a method may further comprise comparing the detected amount of corisin in the biological sample to one or more predetermined thresholds. The predetermined thresholds may be set, e.g., based upon levels of corisin that are typically (normally) present in healthy individuals.

The biological sample may be collected from one or both lungs of the subject.

The biological sample may be, e.g., sputum, bronchial secretion, pleural effusion, bronchoalveolar lavage fluid (BALF), and tissue collected from the bronchus or the lung.

The biological sample may be blood or bronchoalveolar lavage fluid (BALF).

In any of these methods, detection of one of the amino acid sequences of SEQ ID NO: 1, SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, or SEQ ID No: 13 preferably serves as detection of the corisin.

In any of these methods, the patient may have, or be suspected of having or developing, idiopathic pulmonary fibrosis (IPF), liver cirrhosis, kidney fibrosis, cystic fibrosis, myelofibrosis, and/or mammary fibrosis. In particular, the present methods are advantageous for use with patients having idiopathic pulmonary fibrosis (IPF).

The corisin may be detected by mass spectrometry, Western blotting, or enzyme-linked immunosorbent assay (ELISA, e.g., by detecting corisin bound to an antibody that, e.g., recognizes one of the amino acid sequences of SEQ ID NO: 1, SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, or SEQ ID No: 13, e.g., by binding a labeled antibody to the corisin that is bound to an antibody, which is, e.g., bound to a substrate). Kits for performing such a method may include such an antibody and one or more reagents for effecting the detection of the corisin in the biological sample.

In another aspect of the present teachings, a pharmaceutical composition for use in treating fibrosis in a patient is disclosed. The pharmaceutical composition preferably comprises a corisin-inhibitor that is capable of neutralizing corisin in a lung of the patient and/or reducing a quantity of corisin in the lung of the patient.

The corisin-inhibitor may be, e.g., a small molecule, an antagonist of corisin or an antibody to corisin. The corisin-inhibitor may act, e.g., by binding to corisin, by degrading corisin or by blocking or inhibiting the production of corisin.

The corisin-inhibitor may be used to treat patients having, or suspected of having or developing, idiopathic pulmonary fibrosis (IPF), liver cirrhosis, kidney fibrosis, cystic fibrosis, myelofibrosis, and/or mammary fibrosis, in particular idiopathic pulmonary fibrosis (IPF).

In another aspect of the present teachings, a method for identifying a corisin receptor protein may comprise searching for a corisin-binding protein present on a surface of an epithelial cell.

In another aspect of the present teachings, a method for identifying a corisin receptor protein may comprise searching for one of the amino acid sequences of SEQ ID NO: 1, SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, or SEQ ID No: 13 in a binding protein present on a surface of an epithelial cell.

The results of the research that led to the present teachings, as well as a discussion thereof and the particular methods used in the present research are now provided in the following.

### RESULTS

### The Fibrotic Lung Tissue is a Salty Microenvironment

TGFβ1 (transforming growth factor) is considered to be the most important mediator of IPF. Therefore, in the experiments described below in further detail, we used transgenic (TG) mice with lung fibrosis induced by lung overexpression of human TGFβ1, as previously reported, e.g., in NPL8, NPL10, NPL11 and NPL12. Similar to the IPF disease in humans, these TGFβ1 TG mice spontaneously develop pulmonary fibrosis characterized by a predominant and progressive scarring process, fatal outcome and typical lung histopathological findings (diffuse collagen deposition, honeycomb cysts, fibroblast foci-like areas). See NPL8 and NPL11. As controls, we used a line of TGFβ1 TG mice without fibrosis that express the human transgene but not the protein. See NPL8 and NPL13.

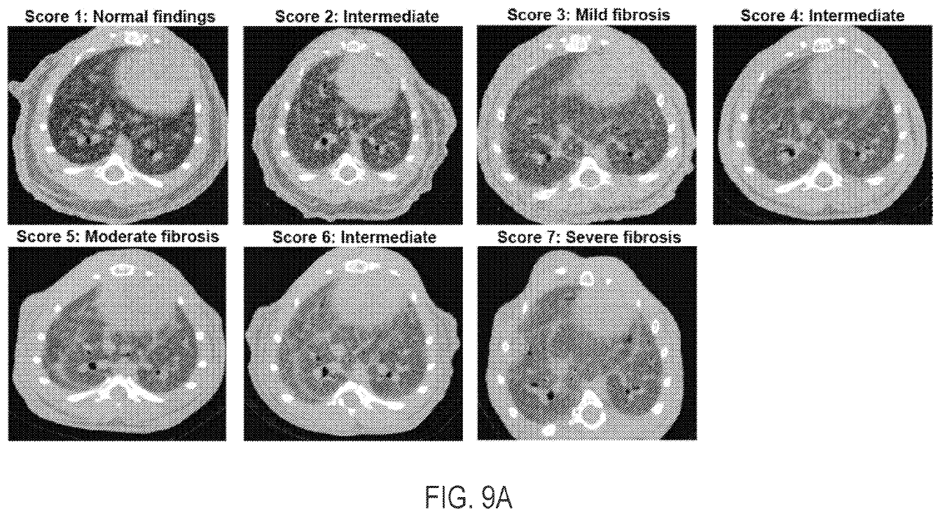

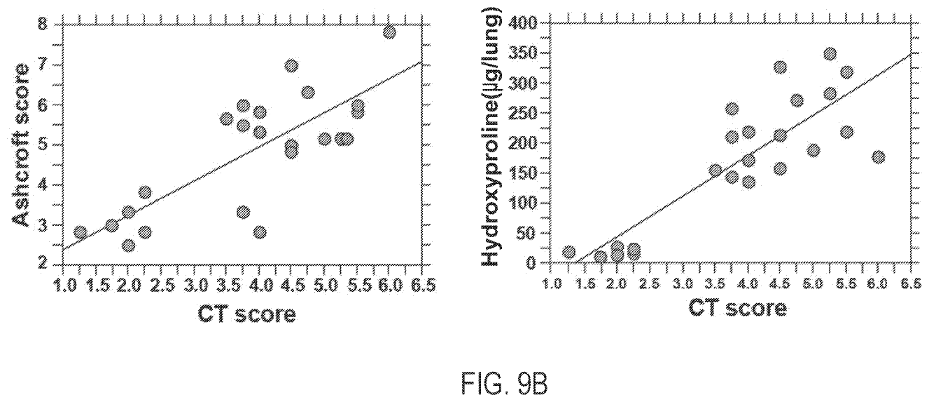

To interrogate the hypothesis that lung fibrotic tissue is a salty microenvironment, we measured the Na⁺ content of lung fibrotic tissues from TGFβ1 TG mice with lung fibrosis (see NPL8), by allocating TGFβ1 TG and wild-type (WT) mice in groups according to computed tomography-based fibrosis scores (see FIGS. 9A and 9B).

More specifically, FIG. 9A shows computed tomography (CT) images that were obtained according to the methods described below. Criteria for scoring CT findings were as follows: score 1: normal findings; score 2, intermediate; score 3; mild fibrosis; score 4: intermediate; score 5, moderate fibrosis; score 6: intermediate; and score 7, severe fibrosis. The average of scores of six pulmonologists was taken as the CT score of an individual mouse.

FIG. 9B shows the Ashcroft fibrosis score and the hydroxyproline contents that were measured according to the methods described below. 10-week old male mice having a body weight of 20 to 25 g were used in the experiments. N=23 mice. The CT score was significantly correlated with the Ashcroft score (r=0.78; p<0.0001) and with the hydroxyproline content of the lungs (r=0.84; p<0.0001). Statistical analysis was performed according to Pearson-product moment correlation.

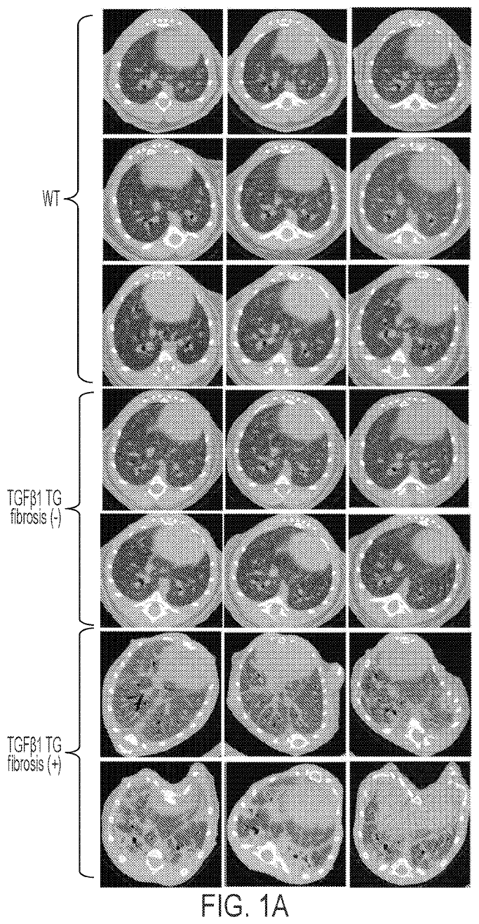

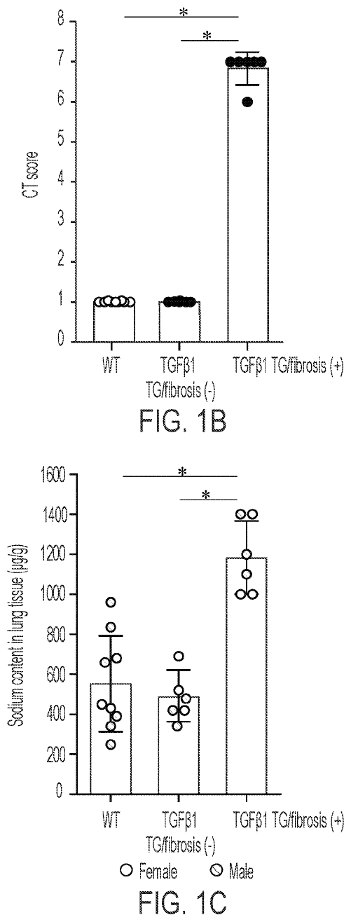

As a result of these experiments, we found there was a significantly higher concentration of Na⁺ in lung tissue from TGFβ1 TG mice with lung fibrosis as compared to TG mice without lung fibrosis and WT mice (see FIGS. 1A-1C). These observations demonstrated that the lung fibrotic tissue is a salty microenvironment.

### Abnormal Immune Response in Lung Fibrotic Tissue

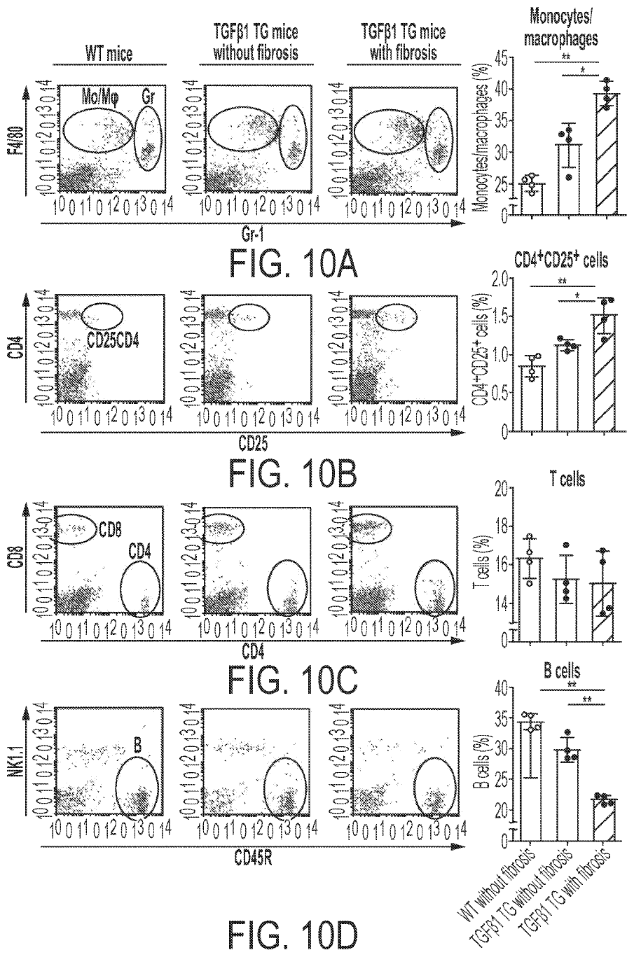

We separated lung immune cells from each of the WT mice without fibrosis, TGFβ1 TG mice without lung fibrosis and TGFβ1 TG mice with fibrosis and compared the percentage of cells between groups. We found a significant increase in the percentage of monocyte/macrophages and regulatory (CD4⁺CD25⁺) T cells in TGFβ1 TG mice with lung fibrosis compared to WT mice and TGFβ1 TG mice without lung fibrosis (See FIGS. 10A and 10B and Table 1 below). Although the percentage of total T cells was not different between groups, the percentage of B cells was significantly decreased in TGFβ1 TG mice with lung fibrosis compared to WT mice and TGFβ1 TG mice without lung fibrosis (See FIGS. 10C and 10D). These observations provided evidence of impaired immune response in lung fibrotic tissue.

More specifically, FIGS. 10A-10D respectively show the percentages of monocytes/macrophages, CD4CD25 cells, T cells and B cells in lung fibrotic tissue from wild-type (WT) mice (n=4) and from TGFβ1 transgenic (TG) mice with (n=4) and without (n=4) fibrosis, which were counted by flow cytometry using specific antibodies as further described in the methods below. Bars indicate the means ±S.D. Statistical analysis was performed using ANOVA with Tukey's test. *p<0.05, **p<0.01.

TABLE 1

| | | TGFβ1 TG mice | TGFβ1 TG mice |
|---|---|---|---|
| Immune cells (%) | WT mice | without fibrosis | with fibrosis |
| Monocytes/Macrophages | 25.00 ± 1.28 | 31.09 ± 3.48* | 39.30 ± 1.93**† |
| Granulocytes | 11.59 ± 1.18 | 11.26 ± 0.89 | 12.54 ± 1.10 |
| Dendritic cells | 7.00 ± 0.30 | 6.86 ± 0.82 | 7.17 ± 0.70 |
| Total lymphocytes | 56.41 ± 1.30 | 50.80 ± 2.73* | 41.00 ± 1.94**‡ |
| B cells | 34.41 ± 1.29 | 29.87 ± 1.98* | 21.67 ± 0.76**‡ |
| T cells | 16.33 ± 1.03 | 15.24 ± 1.23 | 15.02 ± 1.68 |
| Natural killer cells | 5.27 ± 0.51 | 4.80 ± 0.31 | 2.85 ± 0.43**‡ |
| Natural killer T cells | 0.40 ± 0.11 | 0.88 ± 0.42 | 1.45 ± 0.34* |
| CD4⁺ T cells | 9.44 ± 0.18 | 9.11 ± 1.42 | 9.26 ± 0.84 |
| CD8⁺ T cells | 6.75 ± 0.99 | 6.12 ± 0.50 | 6.39 ± 0.53 |
| CD4⁺CD25⁺ | 0.85 ± 0.14 | 1.12 ± 0.07 | 1.52 ± 0.24**† |
| γ/δ T cells | 0.53 ± 0.11 | 0.52 ± 0.11 | 0.74 ± 0.10*† |
| B/T cells ratio | 2.12 ± 0.19 | 1.96 ± 0.03 | 1.46 ± 0.16**† |
| CD4/CD8 ratio | 1.43 ± 0.25 | 1.50 ± 0.32 | 1.45 ± 0.05 |

Data are the means ± S.D. Number of cells are expressed as the percentage of total number of lung cells. Each mouse group had n = 4. Statistical analysis performed by ANOVA with Tukey's test *p < 0.05 or **p < 0.01 vs WT mice; †p < 0.05 or ‡p < 0.05 vs TGFβ1 TG mice without fibrosis. TGFβ1, transforming growth factorβ1. WT, wild type.

### Sodium, Immune Cells, Fibrotic Markers, and Sodium Channels

The lung tissue relative mRNA expression of fibrotic markers (connective tissue growth factor, fibronectin 1, collagen I) and of pro-fibrotic cytokines (TGFβ1, tumor necrosis factor-α, interferon-γ), chemokines (monocyte chemoattractant protein-1), vascular endothelial growth factor or inducible nitric oxide synthase were significantly increased in TGFβ1 TG mice with lung fibrosis compared to WT mice and TGFβ1 TG mice without fibrosis (see Table 2 below).

However, the lung tissue relative mRNA expression of the chloride (cystic fibrosis transmembrane conductance regulator) channels and sodium (Scnnγ, Scnnβ) channels were significantly decreased in TGFβ1 TG mice with lung fibrosis compared to WT mice and TGFβ1 TG mice without lung fibrosis (see Table 2 below). Therefore, we evaluated the correlation between variables in all WT mice and all TGFβ1 TG mice with and without fibrosis.

TABLE 2

| | Variables | | |
|---|---|---|---|
| | | TGFβ1 TG | TGFβ1 TG |
| mRNA relative level | WT | without fibrosis | with fibrosis |
| Ctfr | 0.965 ± 0.057 | 0.720 ± 0.118 | 0.492 ± 0.135*† |
| Scnn1γ | 0.910 ± 0.117 | 0.817 ± 0.117 | 0.495 ± 0.135*† |
| Scnn1β | 1.198 ± 0.212 | 0.971 ± 0.276 | 0.612 ± 0.094* |
| Scnn1α | 0.100 ± 0.317 | 0.995 ± 0.167 | 0.845 ± 0.218* |
| TNFα | 0.486 ± 0.046 | 0.486 ± 0.102 | 0.893 ± 0.084*† |
| IFNγ | 0.745 ± 0.161 | 0.540 ± 0.078 | 1.162 ± 0.187*† |
| Periostin | 0.860 ± 0.1396 | 0.1396 ± 0.911 | 1.099 ± 0.027 |
| Ctgf | 0.822 ± 0.103 | 0.734 ± 0.039 | 1.186 ± 0.026*† |
| mTGFβ1 | 0.558 ± 0.046 | 0.520 ± 0.054 | 0.792 ± 0.067*† |
| Vegf | 0.630 ± 0.114 | 0.542 ± 0.181 | 1.020 ± 0.263*† |
| INOS | 0.718 ± 0.159 | 0.755 ± 0.097 | 1.235 ± 0.057*† |
| Mcp-1 | 0.695 ± 0.154 | 0.754 ± 0.109 | 1.040 ± 0.065*† |
| αSMA | 0.740 ± 0.078 | 0.666 ± 0.093 | 0.837 ± 0.140 |
| Fn1 | 0.801 ± 0.096 | 0.678 ± 0.092 | 1.097 ± 0.129*† |
| Col1α1 | 0.759 ± 0.074 | 0.493 ± 0.080 | 1.069 ± 0.220*† |
| Plasma active TGFβ1 | 97.542 ± 19.136 | 246.165 ± 94.132 | 365.897 ± 58.751* |
| Plasma total TGFβ1 | 1521.586 ± 645.522 | 3856.940 ± 1973.896 | 8086.258 ± 838.130*† |

Data are expressed as the means ± S.D. Each mouse group had n = 4. Statistical analysis was performed by ANOVA with Tukey's test. *p < 0.05 vs WT; †p < 0.05 vs TGFβ1 TG mouse without fibrosis. Ctfr, cystic fibrosis transmembrane conductance regulator; Scnn1γ, sodium channel epithelial 1 γ subunit; Scnn1β, sodium channel epithelial 1 β subunit; Scnn1α, sodium channel epithelial 1 α subunit; TNFα, tumor necrosis factorα; IFNγ, interferonγ; Ctgf, connective tissue growth factor; mTGFβ1, mouse transforming growth factor β1; Vegf, vascular epithelial growth factor; INOS, inducible nitric oxide synthase; Mcp-1, monocyte chemoattractant protein-1; αSMA, αsmooth muscle actin; Fn1, fibronectin 1; Col1α1, collagen 1α1. WT, wild-type; TG, transgenic.

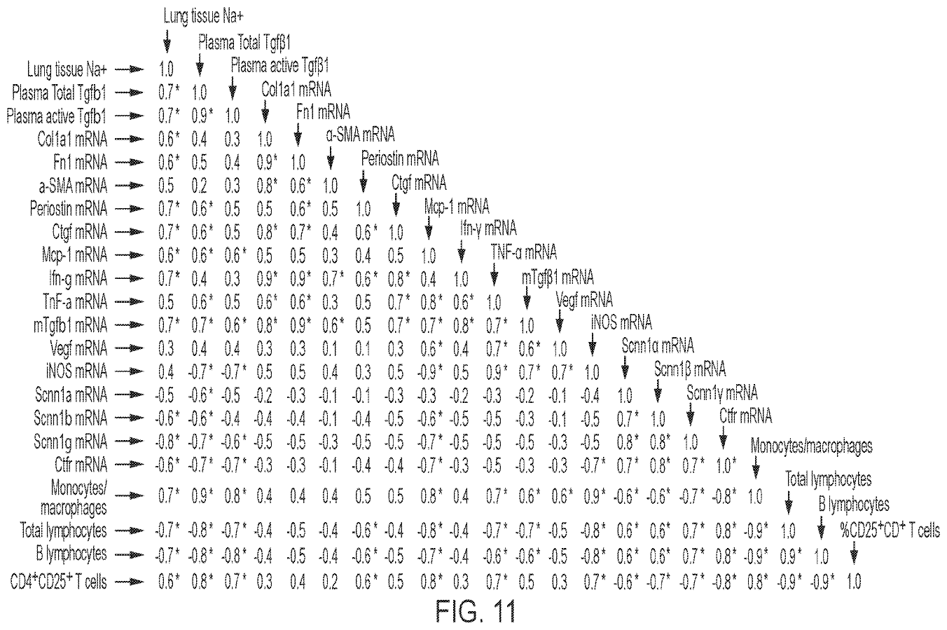

As a result, we found that the tissue level of sodium was inversely and significantly correlated with the mRNA expression of chloride and sodium channels and with the number of B cells. In contrast, the tissue sodium level was proportionally and significantly correlated with fibrotic markers, pro-fibrotic cytokines and with the number of monocytes/macrophages and regulatory T cells (see FIG. 11).

More specifically, the concentration of sodium, the expression of fibrotic factors, pro-fibrotic cytokines, chemokines, angiogenic factors and the percentage of immune cells in lung tissue were assessed in lung tissue from wild-type (n=4) and TGFβ1 TG mice with (n=4) and without (n=4) lung fibrosis. Spearman correlation r values are shown in FIG. 11. Ctfr, cystic fibrosis transmembrane conductance regulator; Scnn1α, sodium channel epithelial 1 α subunit; Scnn1β, sodium channel epithelial 1 β subunit; Scnn1γ, sodium channel epithelial 1 γ subunit; TNFα, tumor necrosis factorα; IFNγ, interferonγ; Ctgf, connective tissue growth factor; mTGFβ1, mouse transforming growth factor β1; Vegf, vascular epithelial growth factor; iNOS, inducible nitric oxide synthase; Mcp-1, monocyte chemoattractant protein-1; αSMA, asmooth muscle actin; Fn1, fibronectin 1; Col1α1, collagen 1α1. Statistical analysis was performed by Spearman correlation. *p<0.05.

These findings provide evidence of the detrimental role of a salty microenvironment in the process of tissue fibrosis and the implication of the tissue sodium level in the regulation of the immune response. See also NPL14.

Growth of Bacteria from Fibrotic Lung Tissue

After confirming that the fibrotic tissue is a salty microenvironment, we posited that a hypersaline culture medium would best mimic the in vivo fibrotic tissue condition, and thus it would favor the growth of microbes implicated in disease pathogenesis.

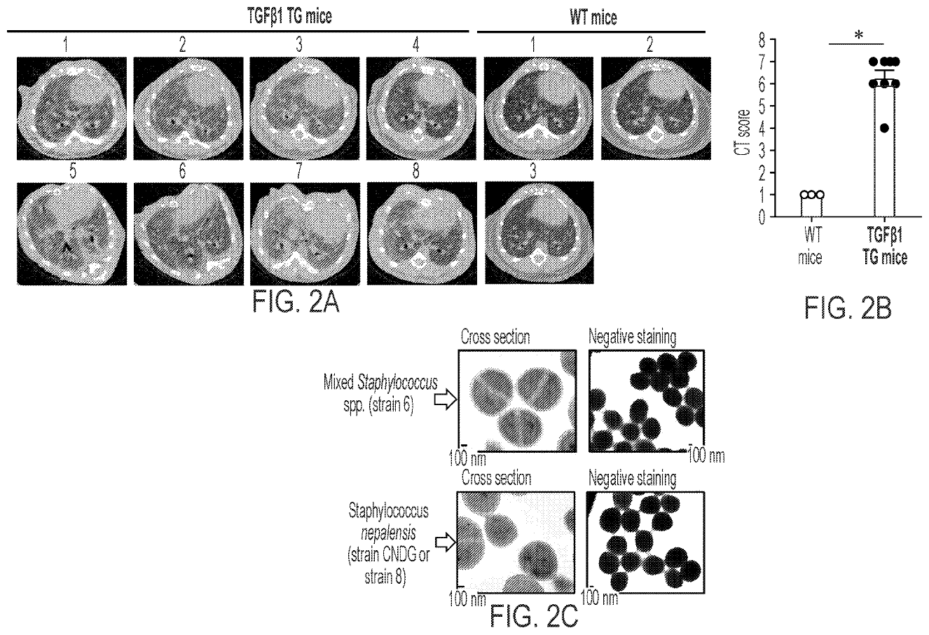

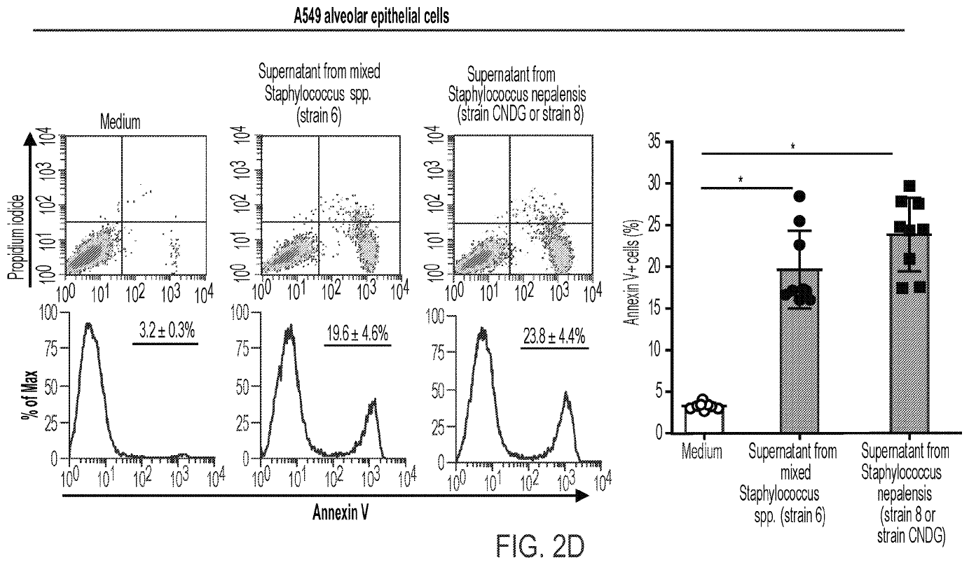

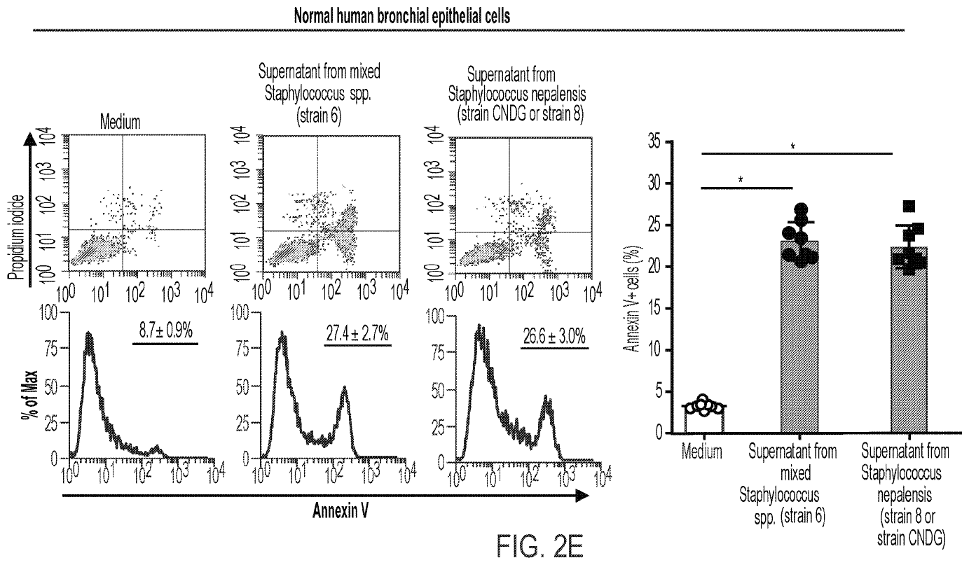

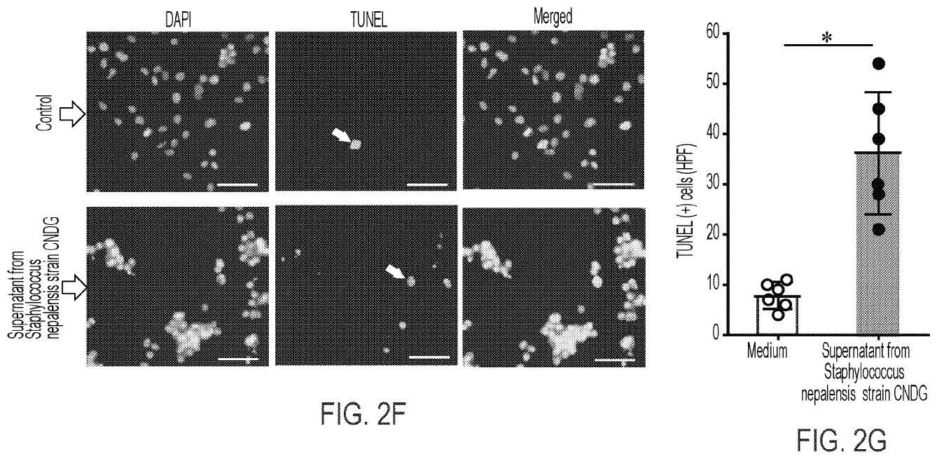

Therefore, we incubated lung fibrotic tissue specimens from TGFβ1 TG and WT mice (see FIGS. 2A and 2B) for 48 h in a medium containing 8% NaCl. Bacterial growth in medium inoculated with lung fibrotic specimens from TGFβ1 TG mice, but not from WT mice, was detected. We then performed streak plating to isolate bacterial colonies, and by using phase-contrast microscopy, a bacteria morphology compatible with Staphylococcus spp. was observed (see FIG. 2C). The identities of the bacterial strains were confirmed by sequencing of their 16S rRNA genes, amplified by polymerase chain reaction.

Determination of the whole genome sequences, however, revealed that while one of the colonies (strain 8) corresponds to a strain of Staphylococcus nepalensis, another colony (strain 6) was a mixture of Staphylococcus spp. The whole genome sequences of the cultures designated strain 6 and strain 8 have been deposited at the Genbank database with the accession number PRJNA544423.

To further confirm the identity of strain 8, we compared its whole genome sequence with that of other Staphylococcus nepalensis strains in the Genbank database, and for strains JS9, SNUC4337, DSM15150, JS11, and JS1; the identities were 99.52%, 99.61%, 99.60%, 99.53% and 99.50%, respectively. Thus, based on the purity of strain 8 and its very high genomic homology to other Staphylococcus nepalensis strains, the bacterium of strain 8 was named Staphylococcus nepalensis with a strain designation of CNDG.

### Apoptosis of Lung Cells Induced by Culture Supernatants

To assess the potential implication of these fibrotic tissue-derived bacterial isolates in disease pathogenesis, we cultured normal human bronchial epithelial (NHBE) cells and A549 alveolar epithelial cells in the presence of the bacterial culture supernatant and evaluated cell survival. Cells cultured in the presence of supernatants from Staphylococcus nepalensis CNDG and the mixed bacteria showed significant levels of apoptosis, caspase-3 activation and DNA fragmentation compared to cells cultured in control medium (see FIGS. 2D-2G).

Culture Supernatant with the Highest Apoptotic Activity

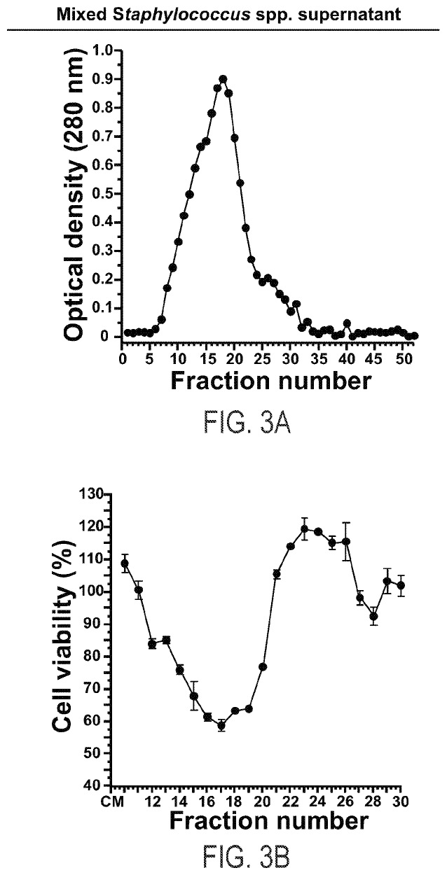

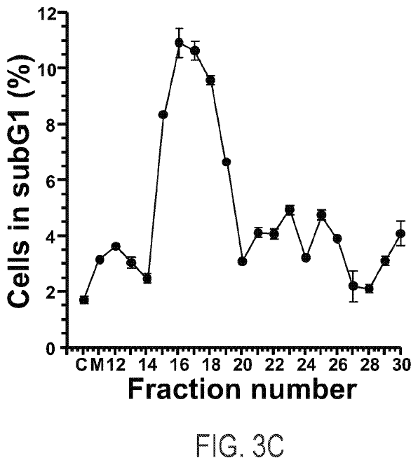

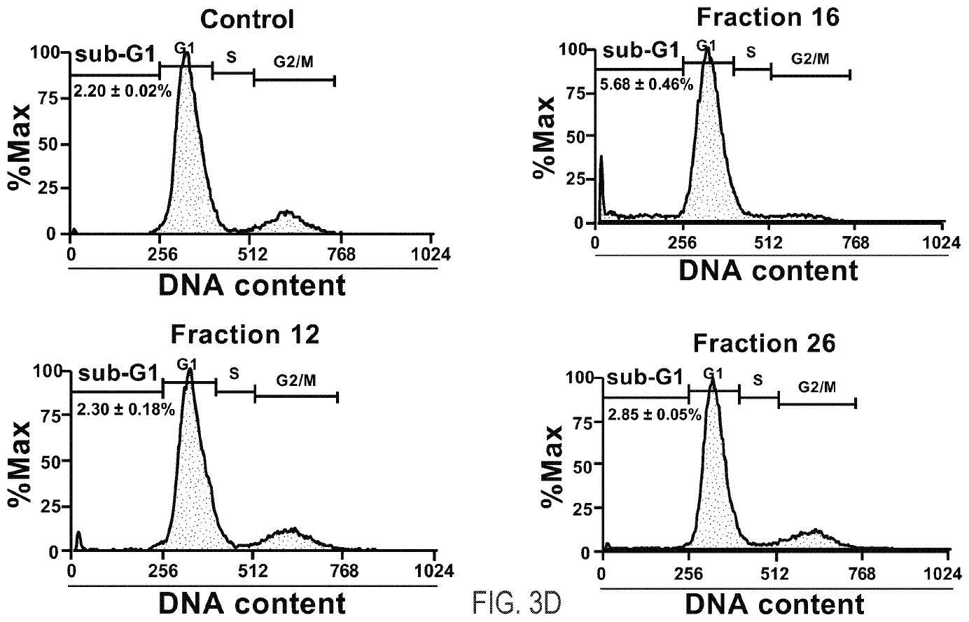

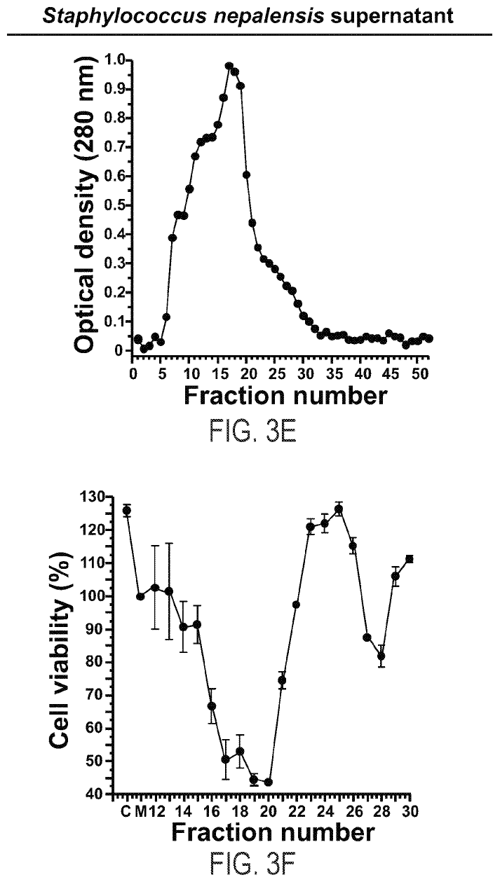

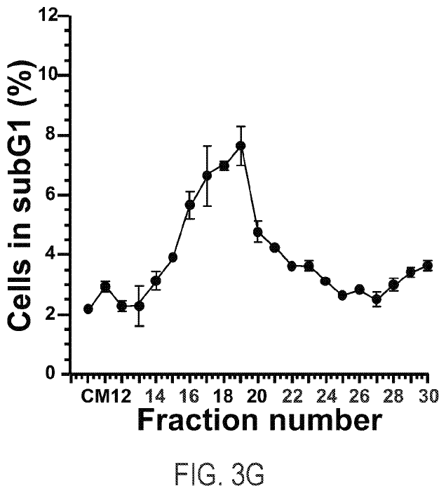

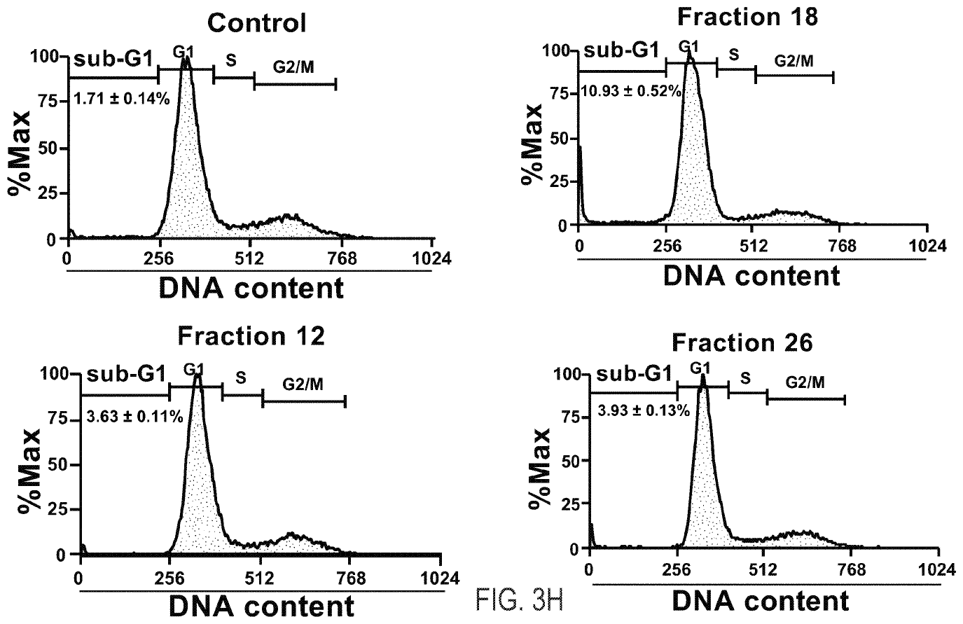

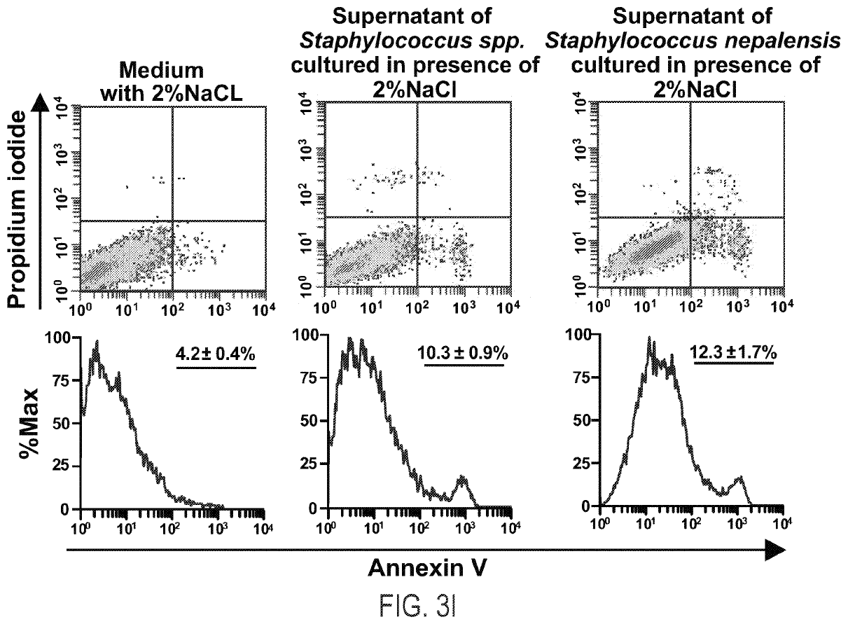

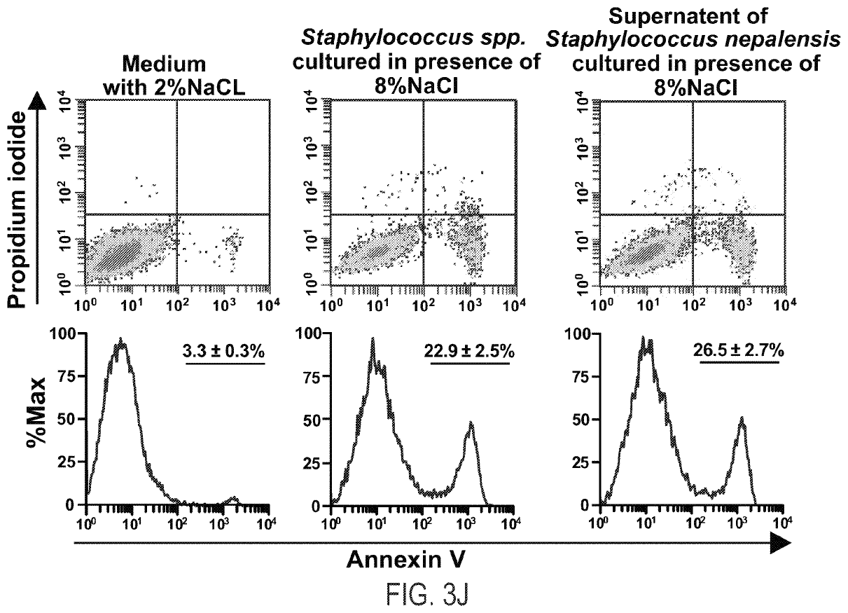

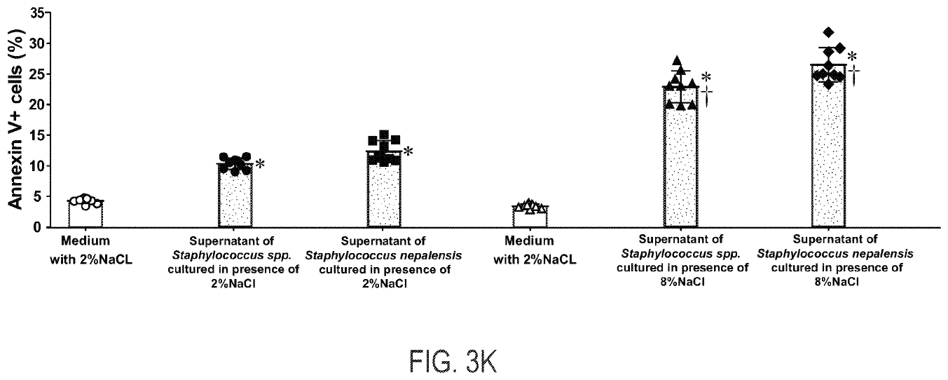

The culture supernatants from the mixed Staphylococcus spp. (strain 6; see FIGS. 3A-3D) and Staphylococcus nepalensis CNDG (strain 8; see FIGS. 3E-3H) were separated into several fractions using a Sephadex column, and the peak of the protein concentrations matched well with the nadir of cell viability of the MTT assay and with the sub-G1 fraction peak of the cell cycle analysis.

### Apoptosis Depends on the Bacterial Medium Salt Concentration

We cultured Staphylococcus nepalensis CNDG and the mixed Staphylococcus spp. in media containing 0%, 2% or 8% NaCl and used the culture supernatant to assess apoptosis by flow cytometry. We found that the apoptotic activity was significantly dependent on the salt concentration of the medium used to culture both isolates in vitro (see FIGS. 3I, 3J and 3K).

### The Apoptotic Factor is a Heat-Stable, Low Molecular Weight Peptide

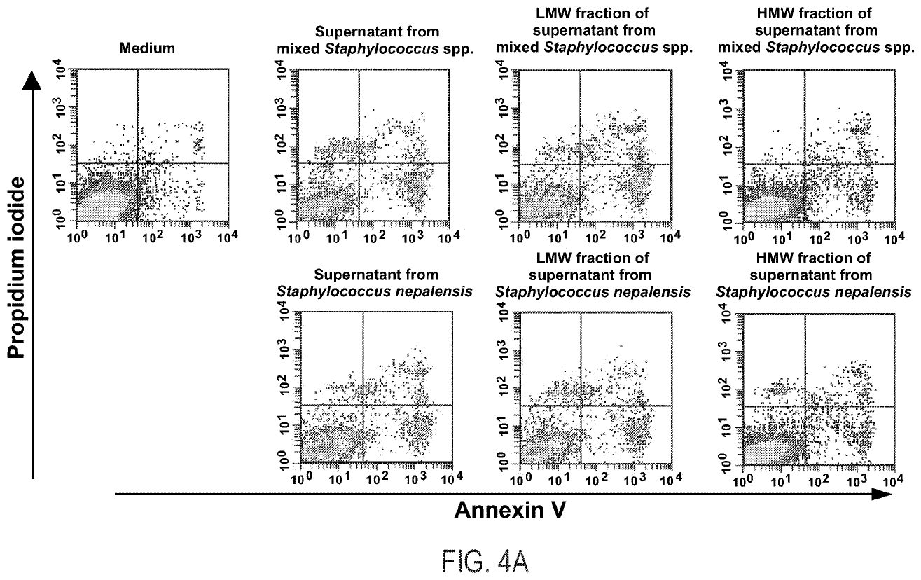

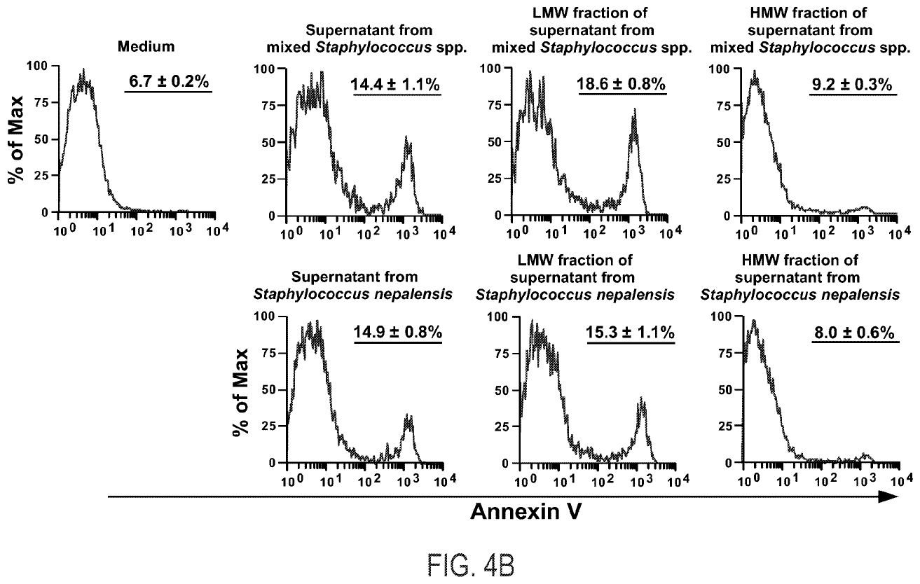

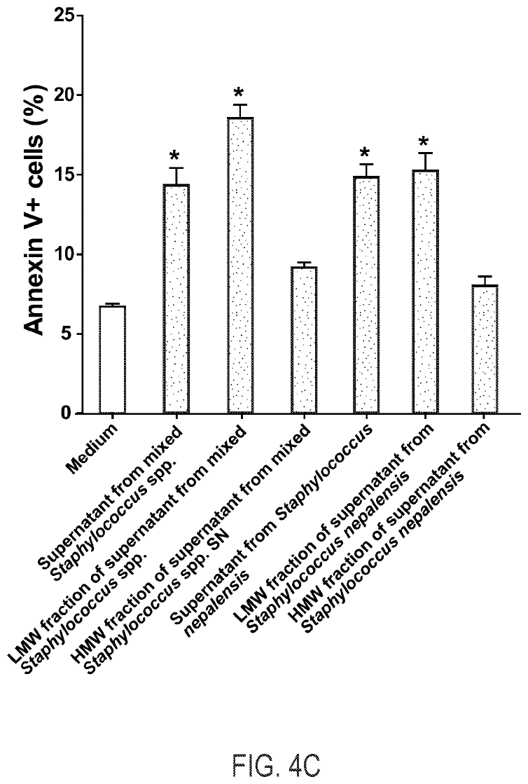

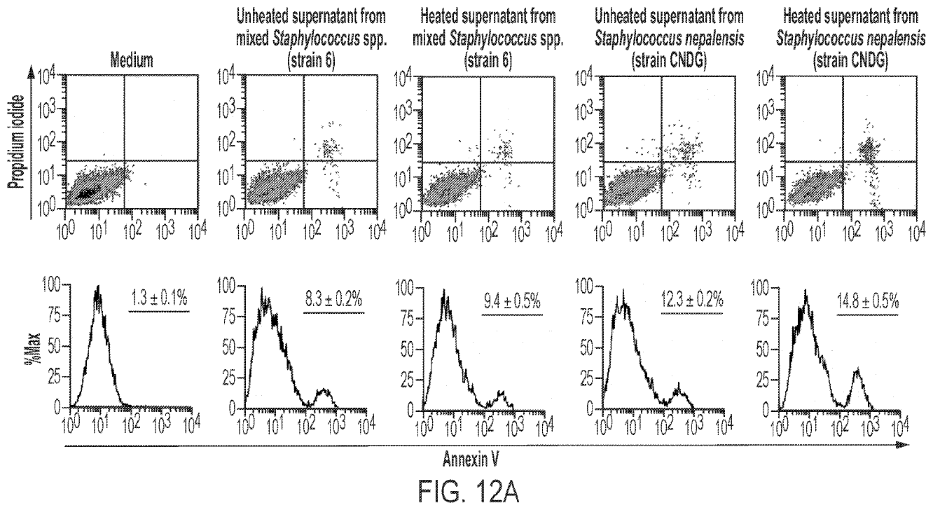

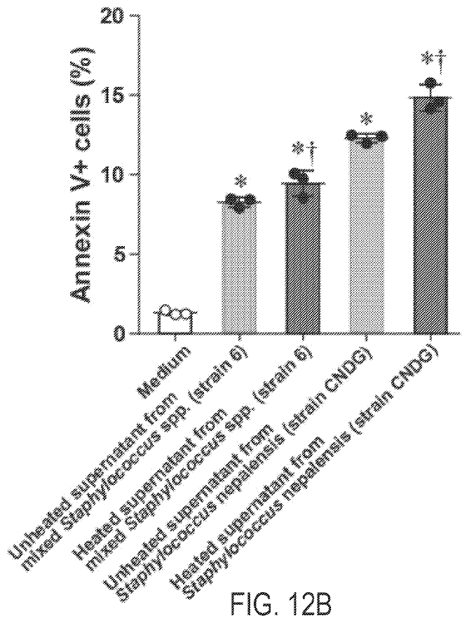

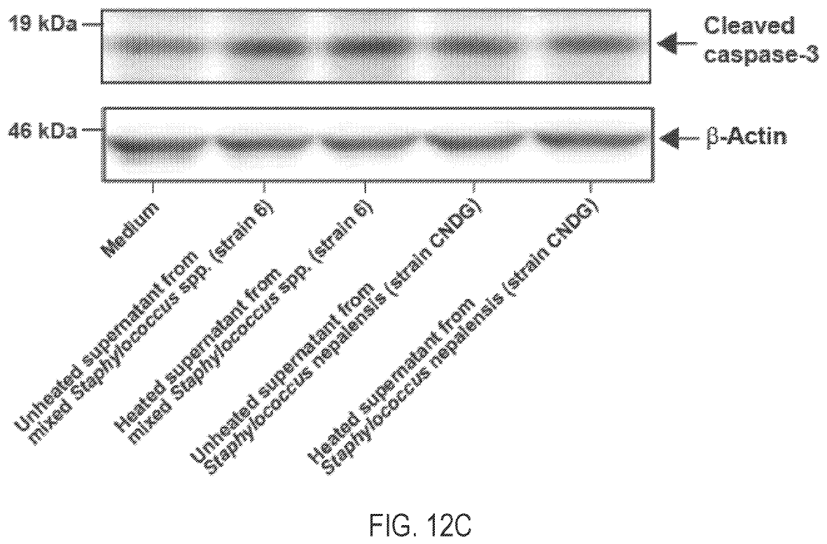

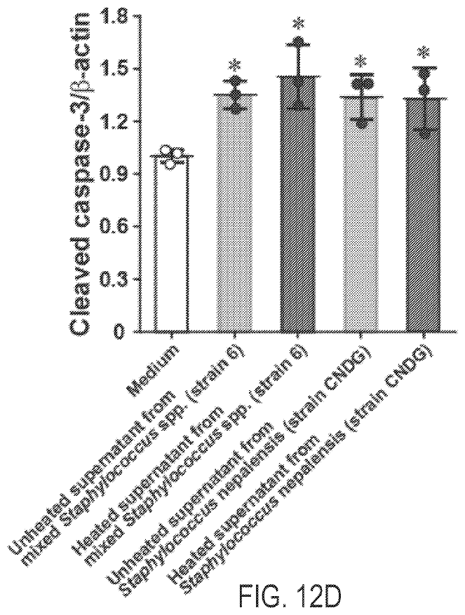

The culture supernatant from bacteria was incubated at 85° C. for 15 min before assessing its pro-apoptotic activity on A549 alveolar epithelial cells at 1/10 dilution. The apoptotic activity of the culture supernatant from both Staphylococcus nepalensis CNDG and the mixed Staphylococcus spp. remained stable after heating, and the activities were significantly stronger than unheated culture supernatant (see FIGS. 12A-12D). To gain insight into the identity of the pro-apoptotic factor, we fractionated the proteins of the bacterial supernatants into low (<10 kDa) and high (>10 kDa) molecular weight proteins, repeated the experiments, and found that the fraction with low-molecular-weight proteins has a potent and significant apoptotic activity compared to the fraction with high-molecular-weight proteins (FIGS. 4A, 4B, 4C, 12A and 12B).

More specifically, FIGS. 12A and 12B show that flow cytometry of A549 cells was performed after staining with propidium iodide and annexin V. Each group had n=3. Bars indicate the means ±S.D. Statistical analysis was performed by ANOVA with Newman-Keuls test. *p<0.001, vs medium; †p<0.05 vs unheated supernatant from Staphylococcus nepalensis (strain CNDG) or from strain 6.

Furthermore, FIGS. 12C and 12D show activation of caspase-3 by the culture supernatant as evaluated by Western blotting after stimulating A549 alveolar epithelial cells in the presence of medium or supernatant of the mixture of Staphylococcus spp. or Staphylococcus nepalensis strain CNDG. Each group with n=3. Bars indicate the means ±S.D. Statistical analysis was performed by ANOVA with Newman-Keuls test. *p<0.05 vs medium.

These observations provided evidence that the apoptosis-inducing factor is a protein of low molecular weight, and that this soluble factor released by the bacteria enriched from the fibrotic tissue contributes to the mechanism of lung fibrosis by sealing the fate of lung epithelial cells.

### Identification of the Pro-Apoptotic Peptide

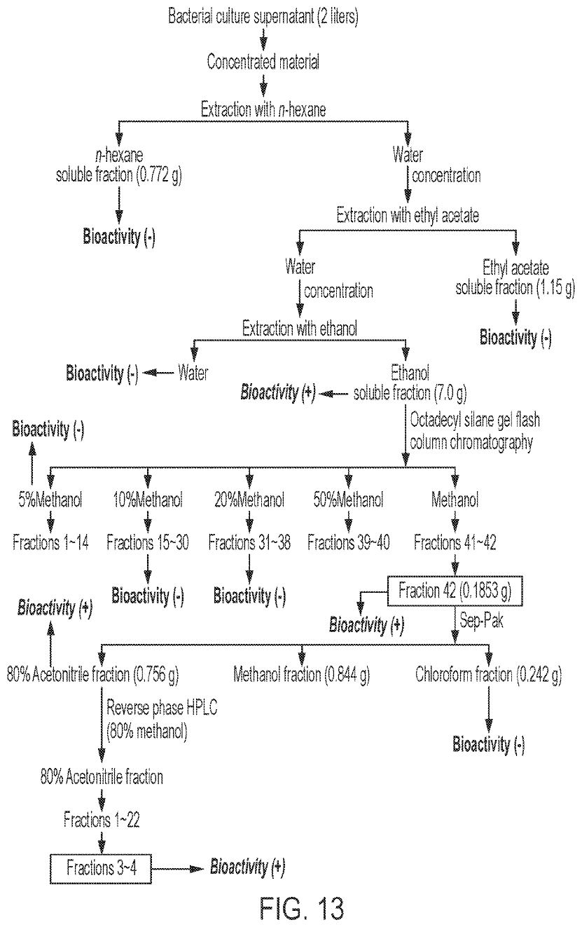

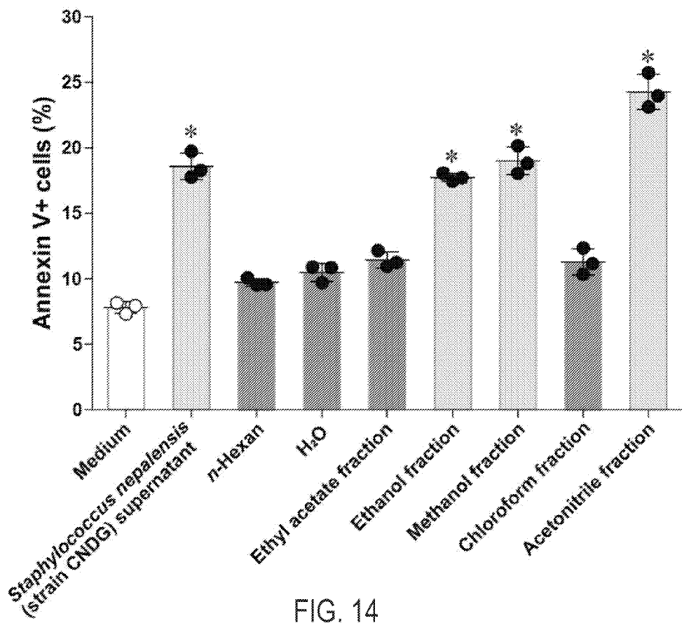

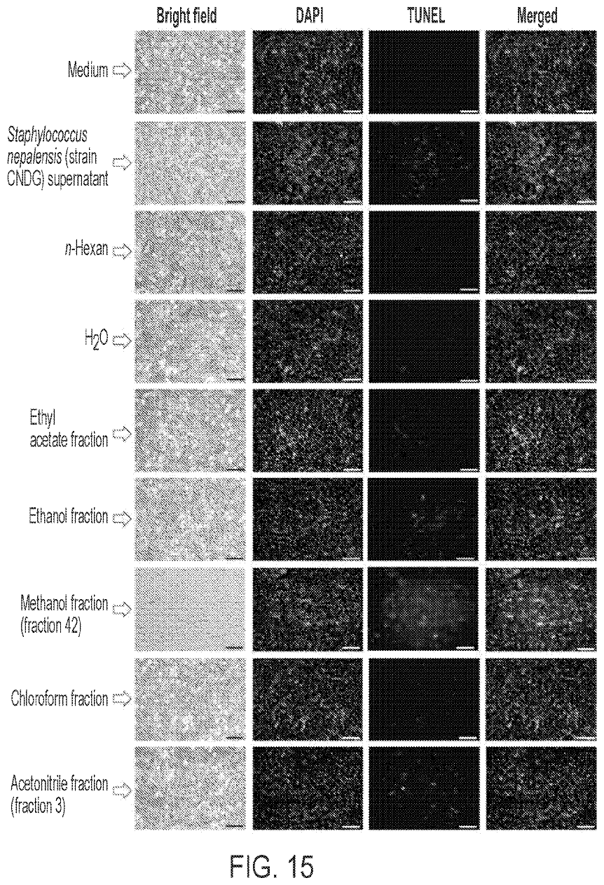

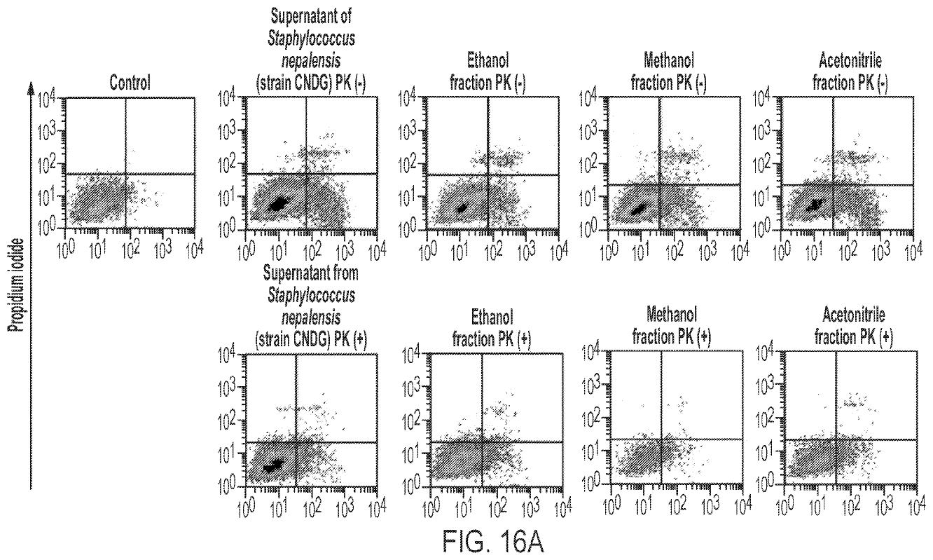

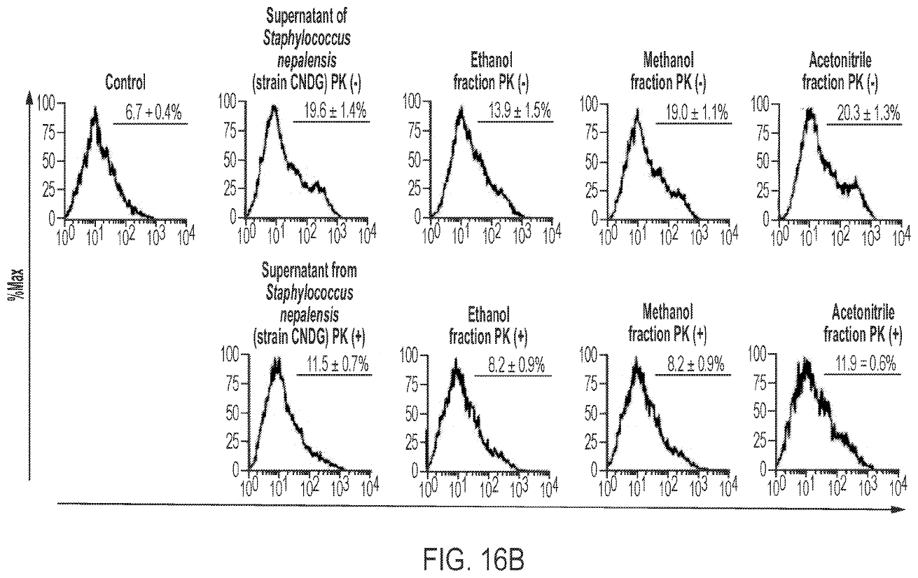

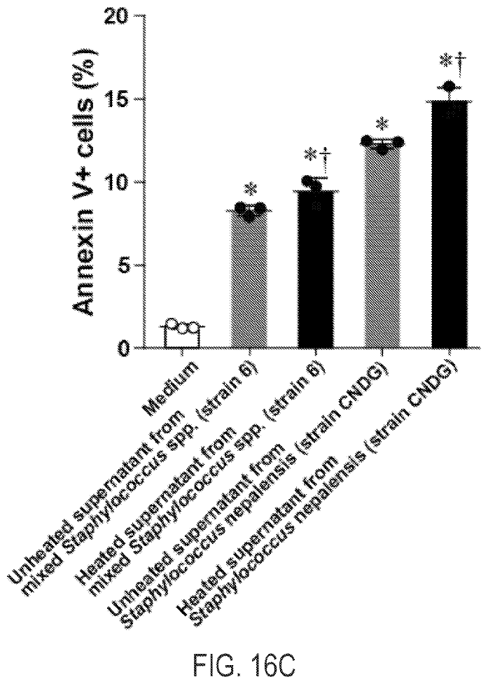

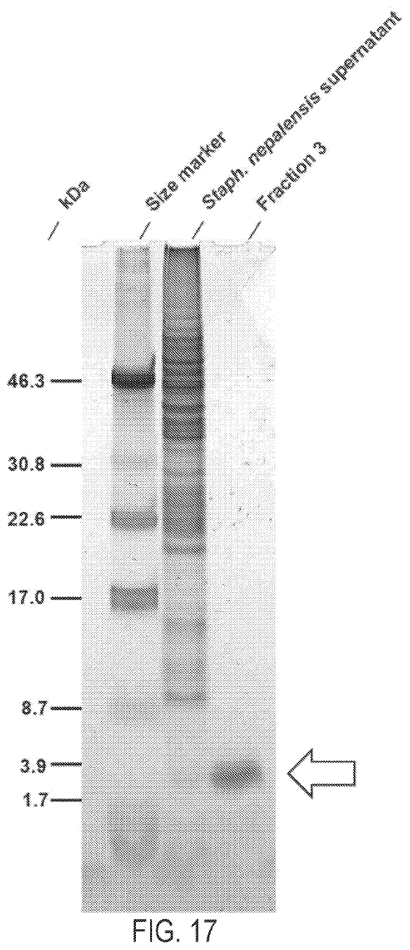

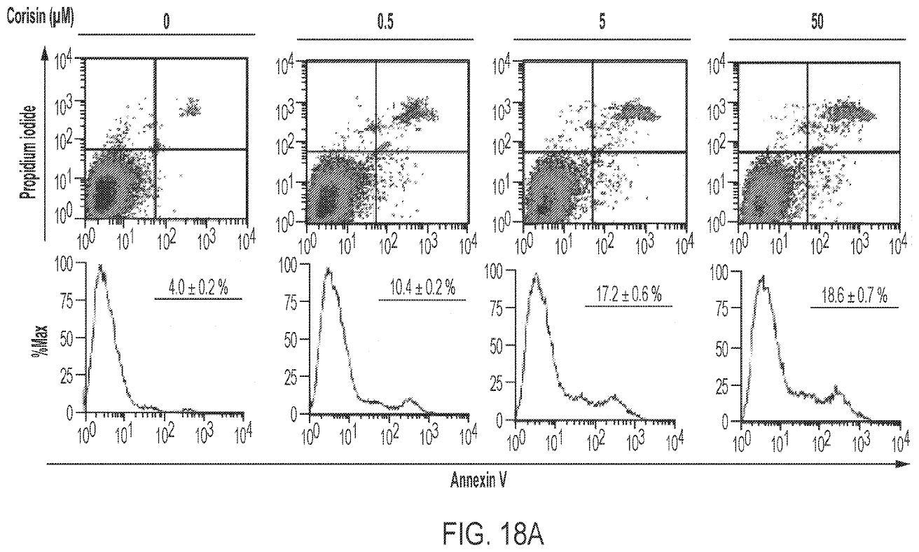

We next proceeded to purify the soluble pro-apoptotic factor from the culture supernatant of Staphylococcus nepalensis strain CNDG. Successive extractions of the proteins in the supernatant were performed in n-hexane, water, ethyl acetate, ethanol and then fractionations using octadecyl-silane gel flash column chromatography and Sep-Pak followed by high-performance liquid chromatography (HPLC) (see FIG. 13) to separate the biologically active protein (see FIGS. 14 and 15). The biological activity decreased significantly after treatment of the samples with proteinase K (see FIGS. 16A, 16B and 16C). Silver staining, after gel electrophoresis of the sample, revealed a protein/peptide with an apparent molecular weight of 2 kDa (see FIG. 17).

More specifically, fractionation of the culture supernatant was performed as described according to the methods below. The pro-apoptotic activity of the fraction on A549 alveolar epithelial cells was evaluated by flow cytometry and it is indicated in FIG. 13 as bioactivity (+) or no bioactivity (−). FIG. 14 shows the pro-apoptotic activity of each of the fractions on A549 alveolar epithelial cells. FIG. 15 shows the pro-apoptotic activity of each of the fractions on A549 alveolar epithelial cells that were cultured in the presence of each fraction for 48 h. Apoptosis was evaluated by a terminal deoxynucleotidyl transferase dUTP nick end labeling (TUNEL) assay, wherein DAPI is an abbreviation of 4′,6-diamidino-2-phenylindole. Representative microphotographs out of two experiments are shown. The scale bars indicate 100 μm.

Culture supernatant as well as ethanol, methanol or acetonitrile fractions of the culture supernatant from Staphylococcus nepalensis were then incubated in the presence of 200 μg/ml of proteinase K (PK) at 37° C. before adding to the culture medium of A549 alveolar epithelial cells at 1/10dilution. Each group had n=3. FIGS. 16A, 16B and 16C show flow cytometry results of A549 alveolar epithelial cells that was performed after staining with propidium iodide and annexin V. Bars indicate the means ±S.D. Statistical analysis was performed by by ANOVA with Tukey's test. *p<0.01. PK is an abbreviation of proteinase K.

Five micrograms of the high-performance liquid chromatography fraction (fraction 3) with biological activity was then loaded on a 15% sodium dodecyl sulfate polyacrylamide gel and silver-staining was performed using a commercial kit. Representative microphotographs out of three experiments with similar results are shown in FIG. 17.

Subsequently, we analyzed the peptide by mass spectrometry and compared the raw data against a custom database of Staphylococcus nepalensis strain CNDG protein sequences, based on its closed genome sequence data (Genbank Accession number PRJNA544423). Mass spectrometry analysis identified a peptide of 19 amino acid residues (IVMPESSGNPNAVNPAGYR—SEQ ID NO.: 1) that corresponded to a molecular mass of 1.94 kDa, in agreement with the purified biological activity in the culture supernatant. We named this newly discovered peptide “corisin”. Homology searching revealed that the corisin sequence corresponds to a segment of transglycosylase 351 IsaA (MW: 25.6 kDa) of Staphylococcus nepalensis strain CNDG.

### Structure Prediction and Apoptotic Activity of Corisin

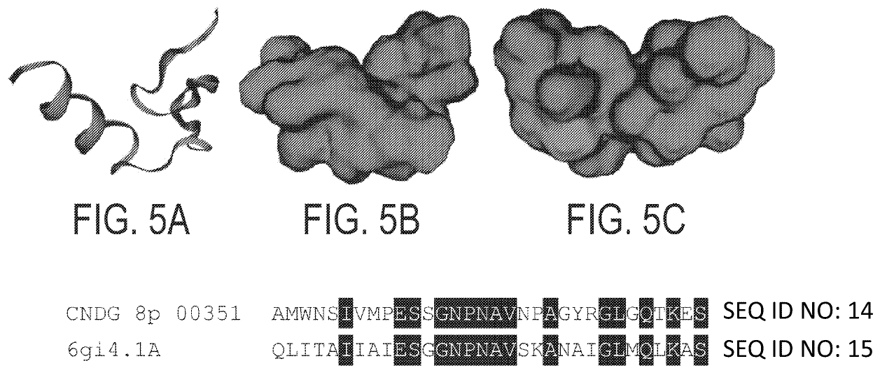

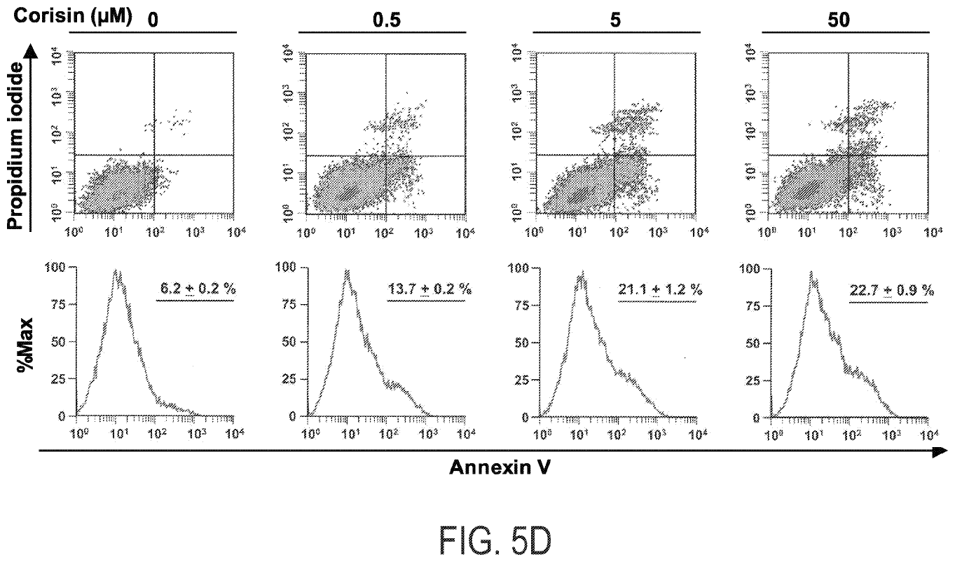

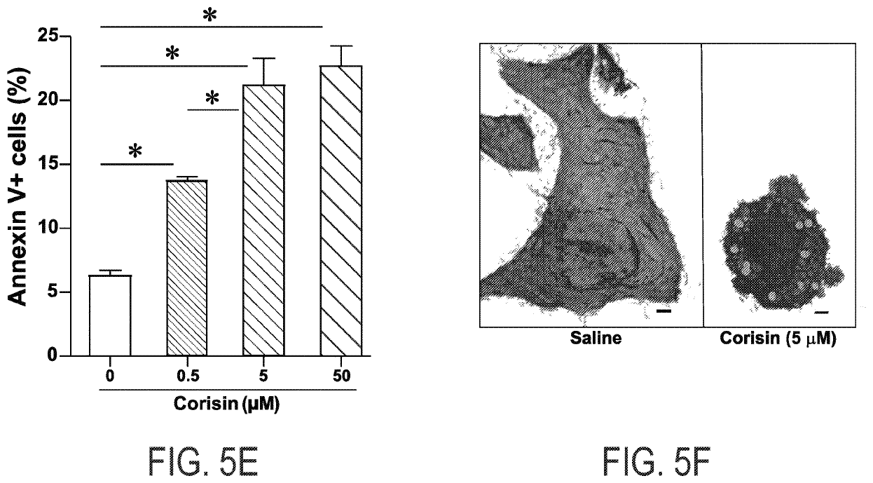

Structural alignment using a homology modelling server (swissmodel.expasy.org) showed that corisin shares 46.88% identity with a segment of an endo-type membrane-bound lytic murein transglycosylase A (see FIGS. 5A-5C). We therefore requested two different commercial manufacturers (Peptide Institute, Osaka, Japan and ThermoFisher Scientific, Waltham, MA, USA) to prepare synthetic corisin peptides (i.e., with the deduced amino acid sequence) for us. Each of these synthetic corisin peptides was then used to treat A549 alveolar epithelial cells.

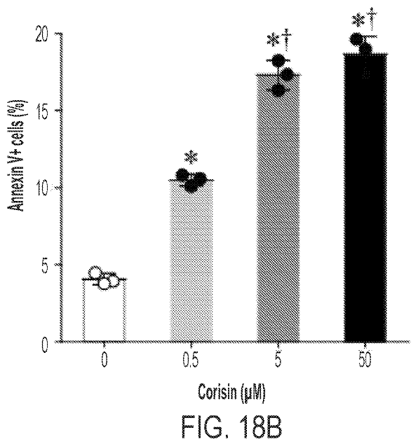

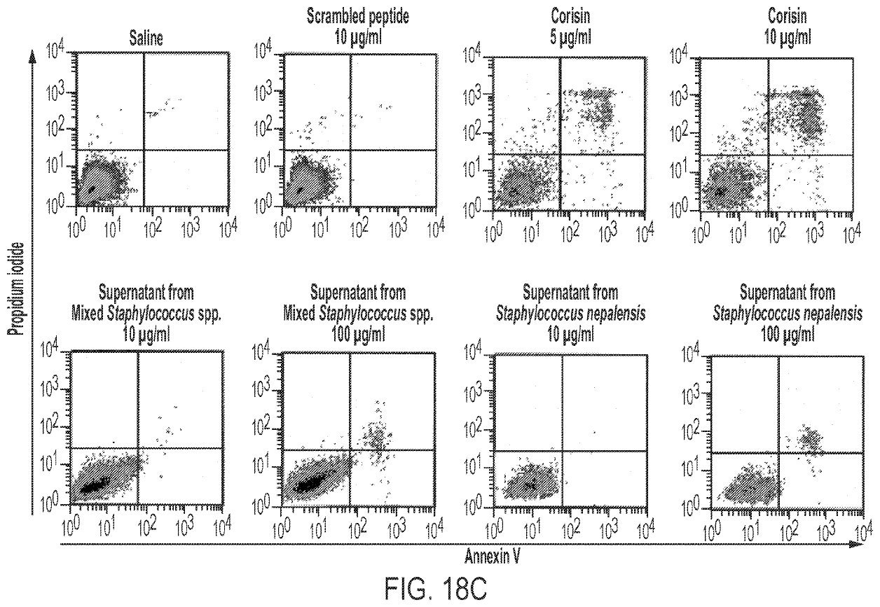

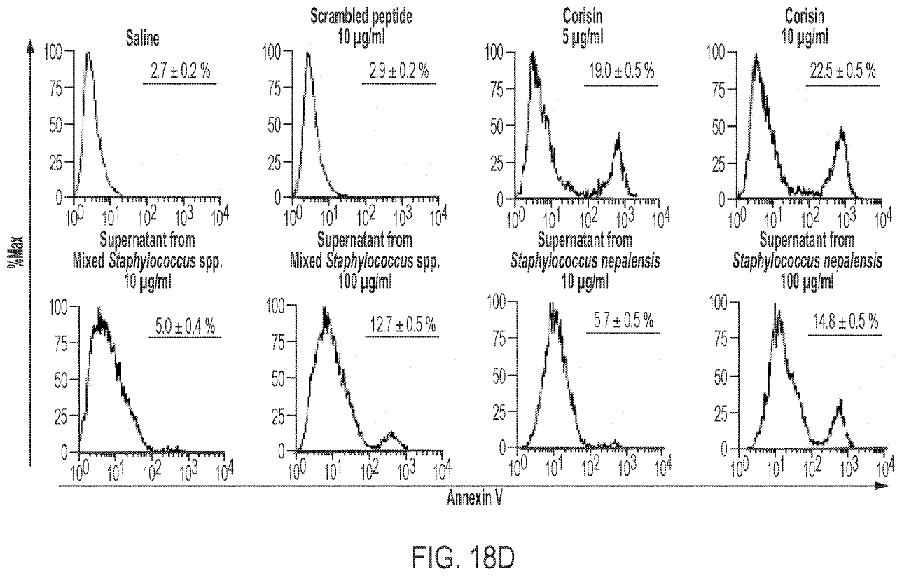

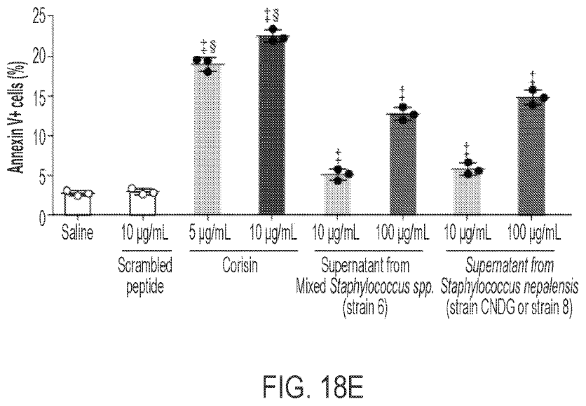

Both synthetic corisin peptides recapitulated the pro-apoptotic effect of the staphylococcal isolate supernatant in a dose dependent manner (see FIGS. 5D, 5E, 18A and 18B) in A549 lung epithelial cells. The apoptotic activity of synthetic corisin was significantly more potent than equal protein concentrations of supernatant from Staphylococcus nepalensis strain CNDG and from the mixed Staphylococcus spp. (strain 6) (see FIGS. 18C-18E).

More specifically, FIGS. 18A and 18B show a flow cytometry analysis of A549 alveolar epithelial cells after culturing for 48 h in DMEM medium containing varying concentration of corisin. Each group had n=3. Bars indicate the means ±S.D. Statistical analysis was performed by ANOVA with Tukey's test. *p<0.001 vs control (0 μg/ml); †p<0.001 vs 0.5 μg/ml of corisin.

FIGS. 18C-18E show a flow cytometry analysis of A549 alveolar epithelial cells after culturing for 48 h in DMEM medium containing varying concentrations of corisin (5 or 10 μg/ml), supernatant protein from mixed Staphylococcus spp. or strain 6 (10 or 100 μg/ml), or from Staphylococcus nepalensis strain CNDG or strain 8 (10 or 100 μg/ml). Each group had n=3. Again, bars indicate the means ±S.D. Statistical analysis was performed by ANOVA and Tukey's test. ‡p<0.05 vs saline or scrambled peptide; §p<0.001 vs supernatant protein (10 and 100 μg/ml) from mixed Staphylococcus spp. or Staphylococcus nepalensis.

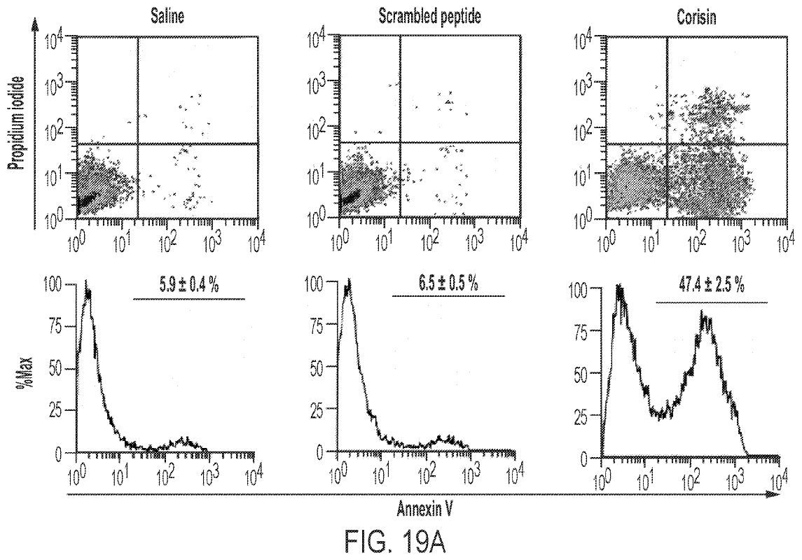

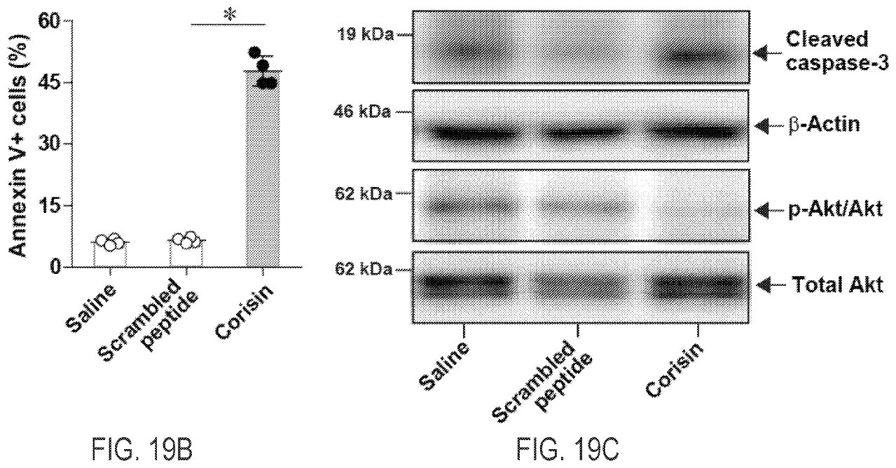

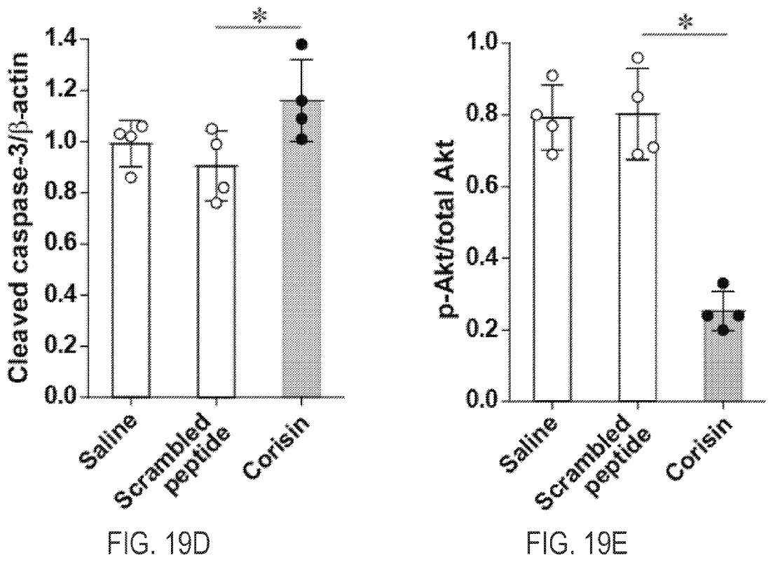

Normal human bronchial epithelial cells also showed significantly enhanced apoptosis in the presence of corisin, but not in the presence of a synthetic peptide composed of a scrambled amino acid sequence (see FIGS. 19A-19B), in association with increased cleavage of caspase-3 and decreased Akt activation (see FIGS. 19C-19E).

More specifically, FIGS. 19A-19B show a flow cytometry analysis of normal human bronchial epithelial (NHBE) cells after culturing for 48 h in DMEM medium containing 10 μM of corisin or of its scrambled sequence. Each treatment group had n=4. Bars indicate the means ±S.D. Statistical analysis by ANOVA with Tukey's test. *p<0.001.

FIG. 19C shows Western blotting of lysates of NHBE cells treated with corisin or scrambled peptide. Each treatment group had n=4. A representative blot of each treatment group is shown.

FIGS. 19D and 19E show the intensity of the Western blot membrane bands as quantified by densitometry using Image? software. Each treatment group had n=4. Bars indicate the means ±S.D. Statistical analysis was performed by one-tailed Mann-Whitney U test. *p<0.05.

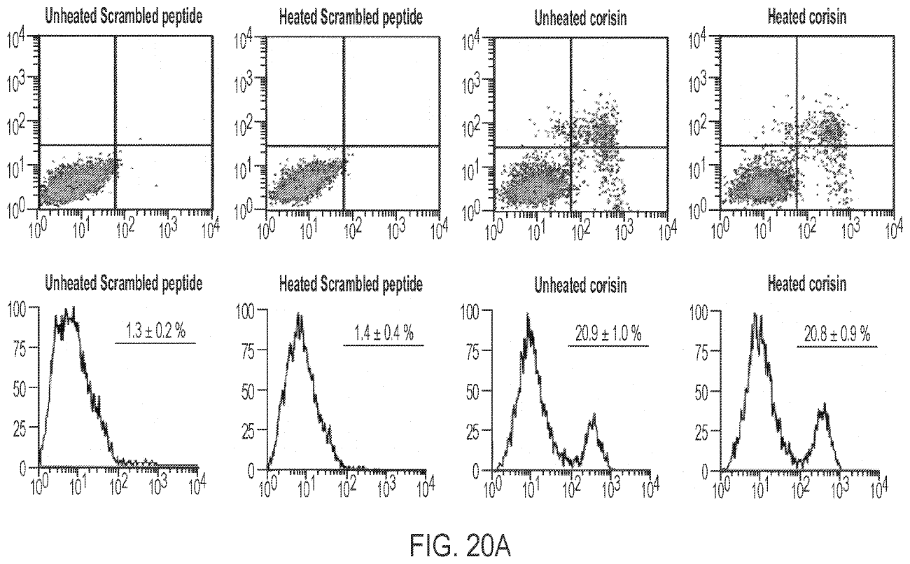

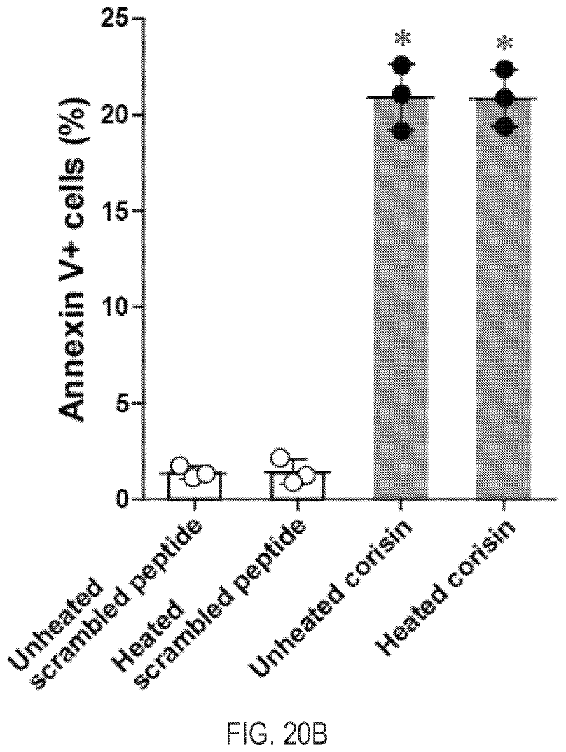

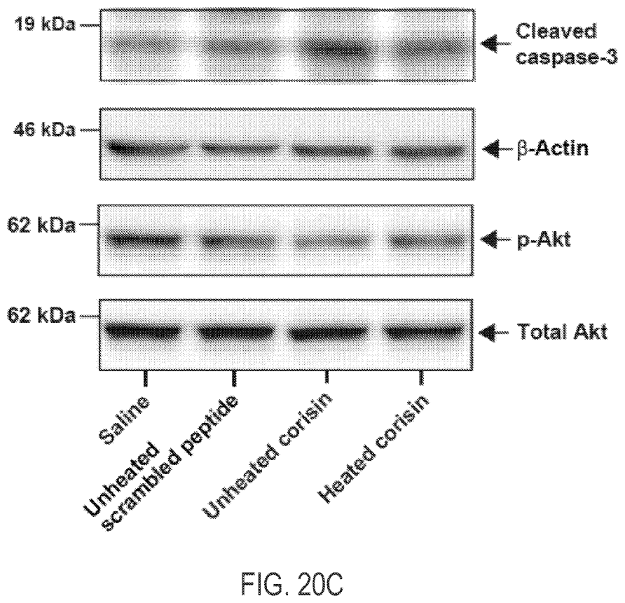

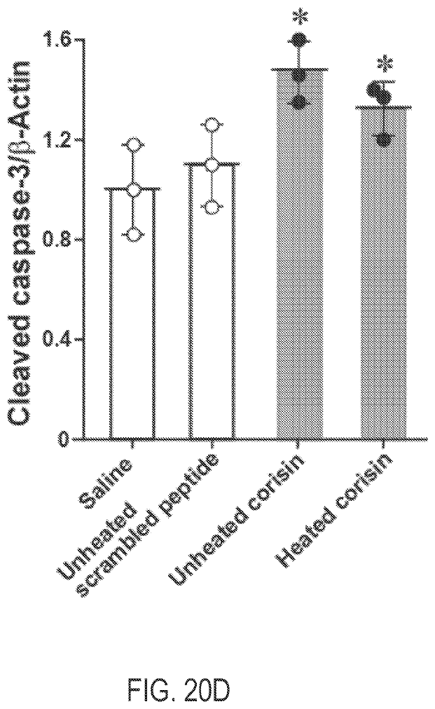

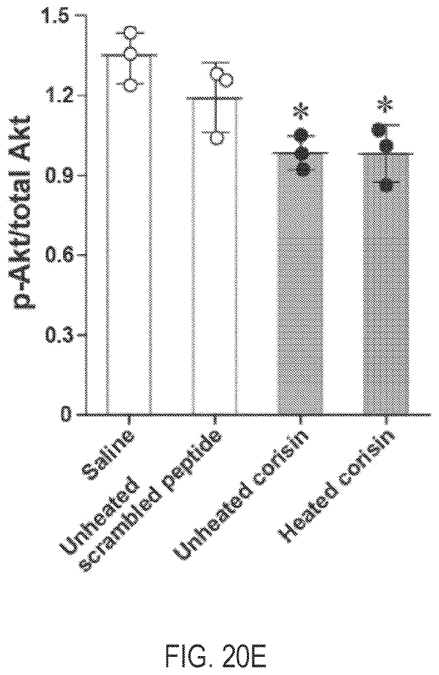

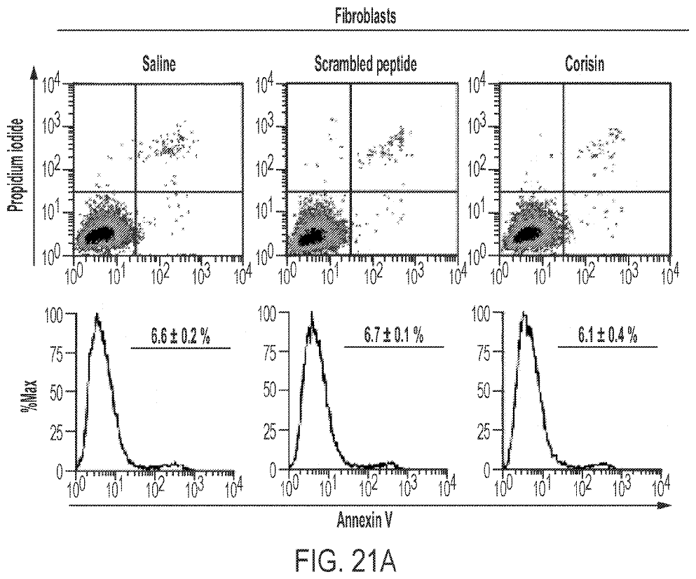

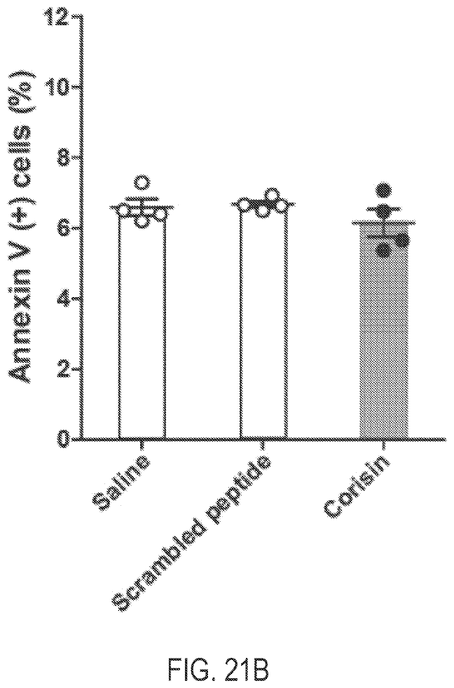

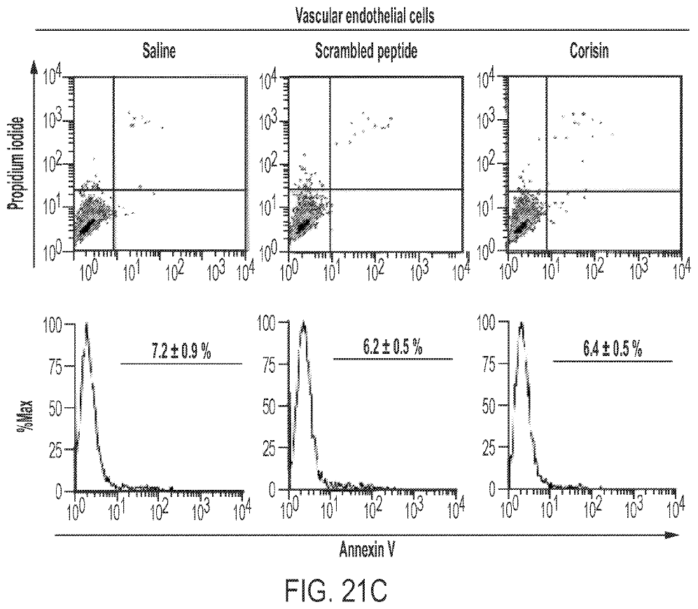

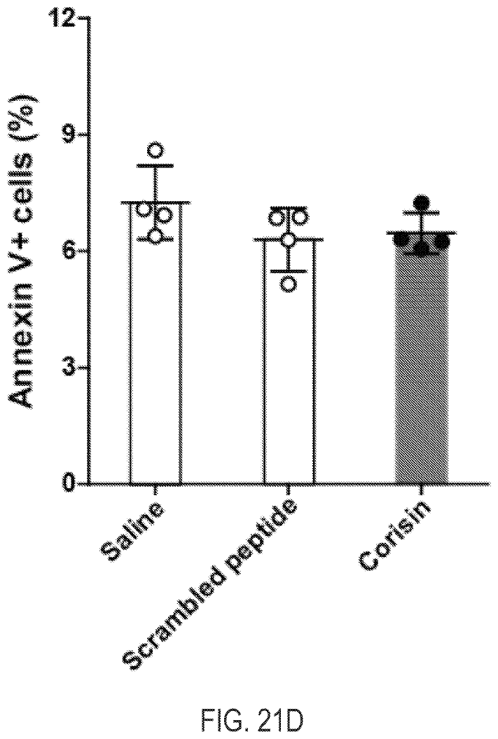

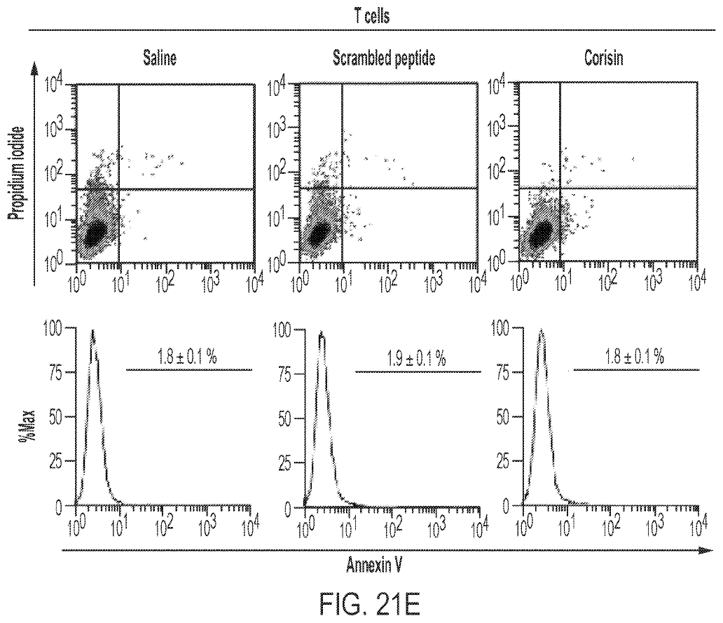

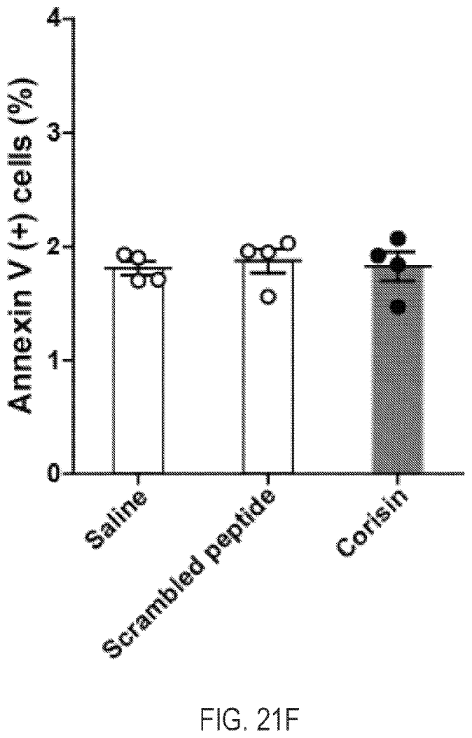

In additional experiments using A549 alveolar epithelial cells, the pro-apoptotic activity of synthetic corisin was found to be heat-resistant (see FIGS. 20A-20B), as observed in the culture supernatant, and examination by transmission electron micrographs confirmed the apoptotic property of corisin (see FIG. 5F). However, corisin showed no apoptotic activity on lung fibroblast, vascular endothelial cell or lymphocyte cell lines (see FIGS. 21A-21F).

More specifically, the synthetic corisin (5 μM; Peptide Institute Incorporation) or scrambled peptide (5 μM; Peptide Institute Incorporation) was incubated at 85° C. for 15 min before adding to the culture medium of A549 alveolar epithelial cells for 48 h. FIGS. 20A-20B show a flow cytometry analysis of A549 alveolar epithelial cells that was performed after staining with propidium iodide and annexin V. Each treatment group had n=3. Bars indicate the means ±S.D. Statistical analysis was performed by ANOVA with Newman-Keuls test. *p<0.001 vs unheated or heated scrambled peptide.

FIG. 20C shows a separate experiment, in which the synthetic corisin (5 μM) or scrambled was incubated at 85° C. for 15 min before adding to the culture medium of A549 alveolar epithelial cells for 48 h, and the cells were collected and prepared for Western blotting of cleaved caspase-3, β-actin, total Akt, phosphorylated Akt (p-Akt). Each treatment group had n=3. A representative blot of each treatment group is shown.

FIGS. 20D and 20E show the intensity of the Western blot membrane bands as quantified by densitometry using the ImageJ software. Each treatment group had n=3. Bars indicate the means ±S.D. Statistical analysis was performed by ANOVA with Newman-Keuls test. *p<0.01 vs saline.

FIGS. 21A and 21B show a flow cytometry analysis of HFL1 lung fibroblasts after culturing for 48 h in DMEM medium containing 10 μg/ml corisin. Each had with n=4.

FIGS. 21C and 21D show a flow cytometry analysis of human umbilical vein endothelial cells after culturing for 48 h in DMEM medium containing 10 μg/ml corisin. Each group had n=4.

FIGS. 21E and 21F show a flow cytometry analysis of human Jurkat T cells after culturing for 48 h in DMEM medium containing 10 μg/ml corisin. Each treatment group had n=4. Bars indicate the means ±S.D. Statistical analysis was performed by ANOVA with Tukey's test.

### Anti-Corisin Antibody Inhibits Corisin-Induced Apoptosis

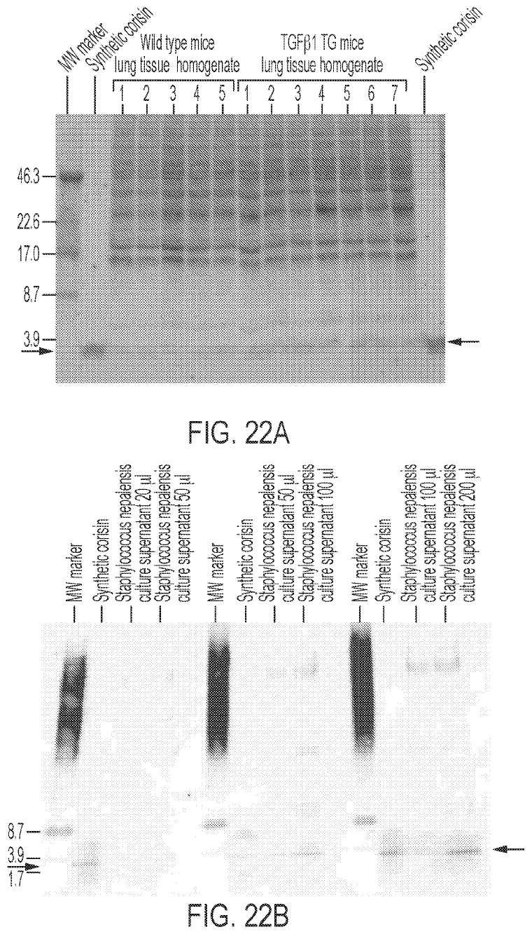

We then developed polyclonal antibody against corisin using the methods described further below. The polyclonal antibody could detect corisin in mouse lung tissue and in culture supernatant of Staphylococcus nepalensis (see FIGS. 22A-22B).

More specifically, five micrograms of lung tissue homogenate prepared from WT mice and TGFβ1 TG mice (FIG. 22A), and several volumes of culture supernatant from Staphylococcus nepalensis (FIG. 22B) concentrated by precipitation with trichloroacetic acid were loaded on a 5-15% gradient sodium dodecyl sulfate polyacrylamide gel, and then Western blotting was performed using anti-corisin antibody. Representative microphotographs out of two experiments with similar results are shown in FIGS. 22A and 22B. Synthetic corisin was used as control. MW is an abbreviation of molecular weight in kDa. Arrows indicate the band of corisin.

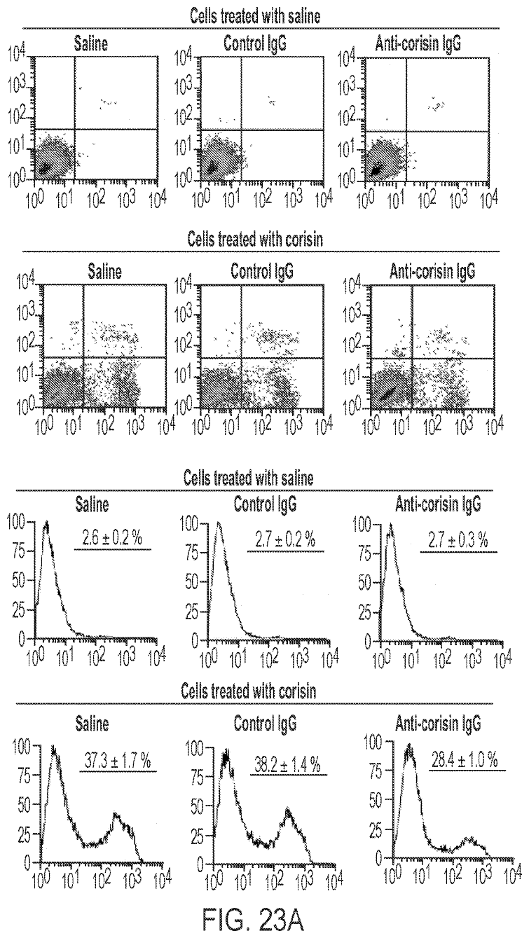

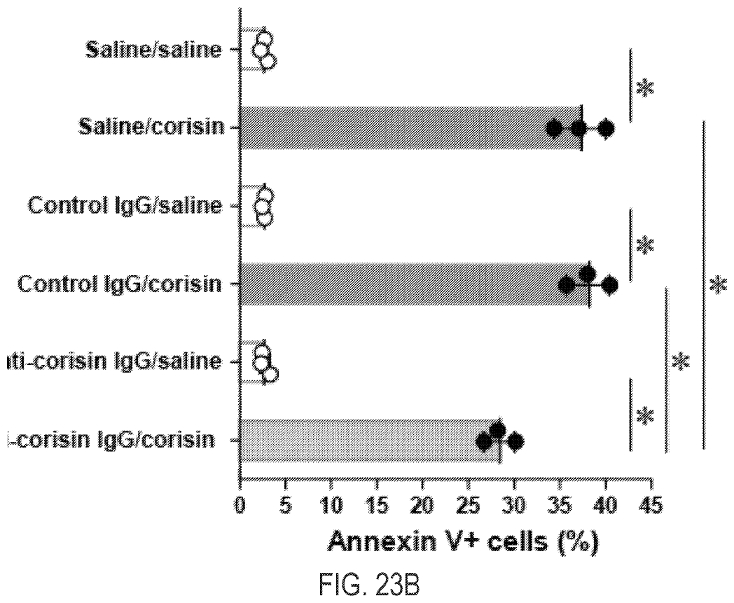

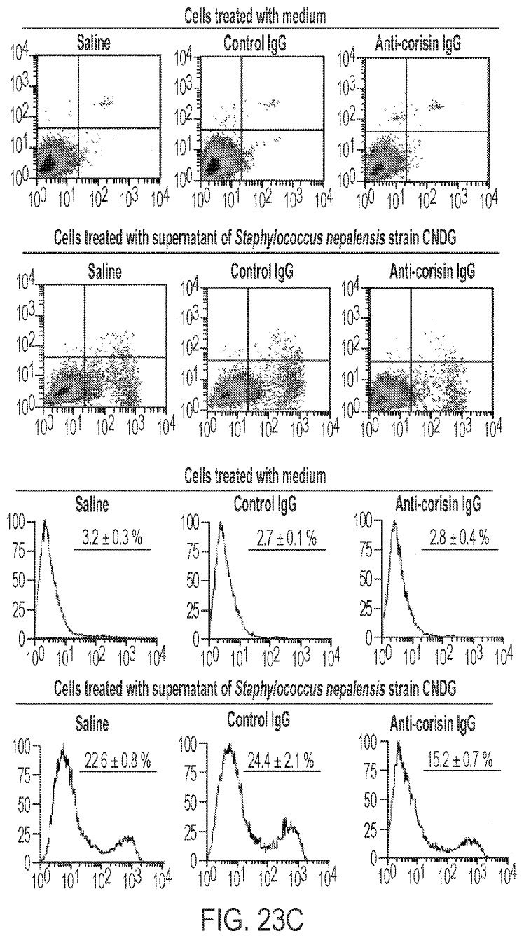

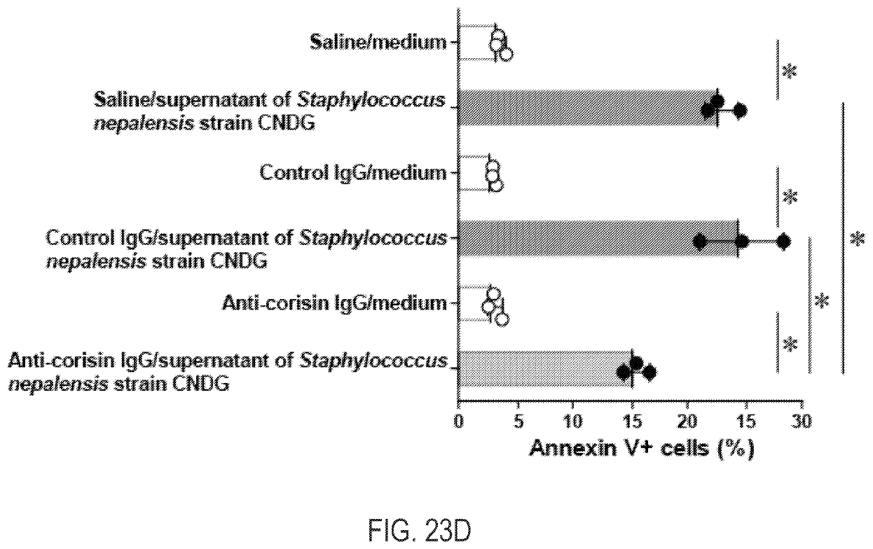

We then stimulated A549 alveolar epithelial cells with corisin or with culture supernatant from Staphylococcus nepalensis strain CNDG in the presence of saline, control rabbit IgG or rabbit anti-corisin IgG and assessed apoptotic cells by flow cytometry. We found significant inhibition of lung epithelial cell apoptosis induced by synthetic corisin (see FIGS. 23A-23B) and by the culture supernatant of Staphylococcus nepalensis (see FIGS. 23C-23D) in the presence of polyclonal anti-corisin antibody as compared to control IgG.

More specifically, A549 alveolar epithelial cells (2×10⁵ cells/well) were cultured in 12-well plates and stimulated with 5 μM corisin in the presence of saline (Saline/corisin), 10 μg/ml control rabbit IgG (Control IgG/corisin) or 10 μg/ml rabbit anti-corisin IgG(Anti-corisin IgG/corisin) for 48 h. Cells cultured in the presence of saline and treated with saline (Saline/saline), control rabbit IgG (Control IgG/saline) or rabbit ant-corisin IgG (Anti-corisin IgG/saline) were used as controls. Each treatment group with n=3 (triplicates). The results are shown in FIGS. 23A and 23B. Bars indicate the means ±S.D. Statistical analysis was performed by by ANOVA with Tukey's test. *p<0.001.

In addition, A549 alveolar epithelial cells cultured in 12-well plates were stimulated with the 1/10 dilution of the culture supernatant of Staphylococcus nepalensis strain CNDG in the presence of saline (Saline/supernatant of Staphylococcus nepalensis strain CNDG), 10 μg/ml control rabbit IgG (Control IgG/supernatant of Staphylococcus nepalensis strain CNDG) or 10 μg/ml rabbit anti-corisin IgG (Anti-corisin IgG/supernatant of Staphylococcus nepalensis strain CNDG) for 48 h. Cells cultured in medium and treated with saline (Saline/medium), control rabbit IgG (Control IgG/medium) or rabbit ant-corisin IgG (Anti-corisin IgG/medium) were used as controls. Each treatment group had n=3. Flow cytometry of A549 cells was performed after staining with propidium iodide and annexin V. The results are shown in FIGS. 23C and 23D. Again, bars indicate the means ±S.D. Statistical analysis was performed by ANOVA with Tukey's test. *p<0.001.

### The Full-Length Transglycosylase has no Apoptotic Activity

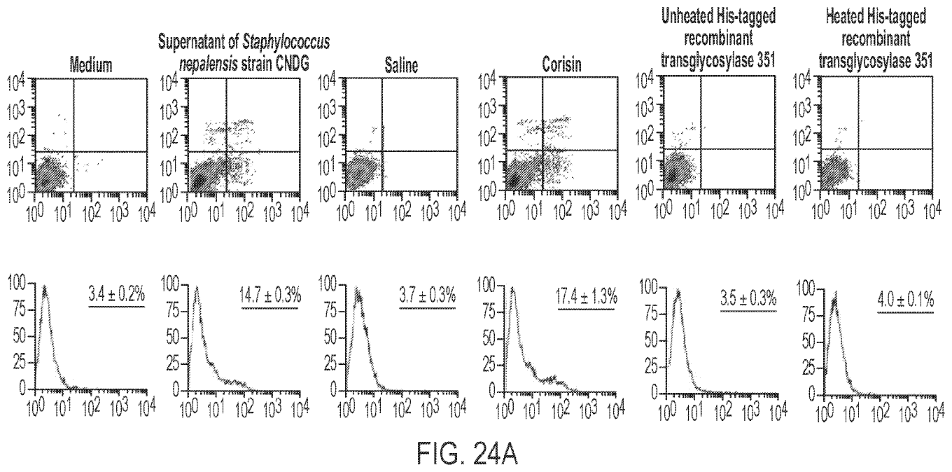

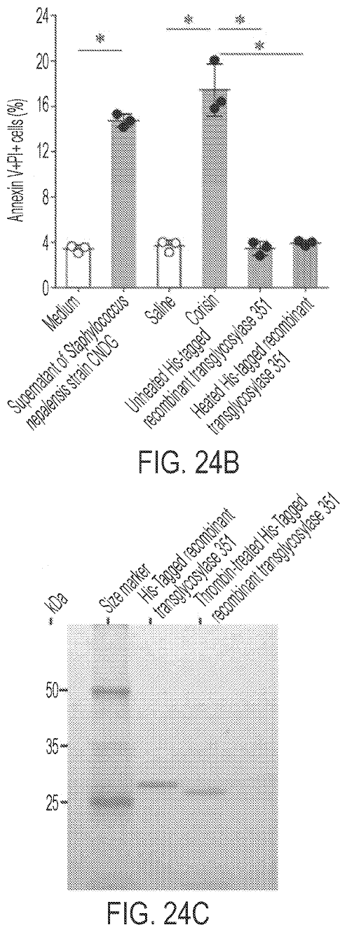

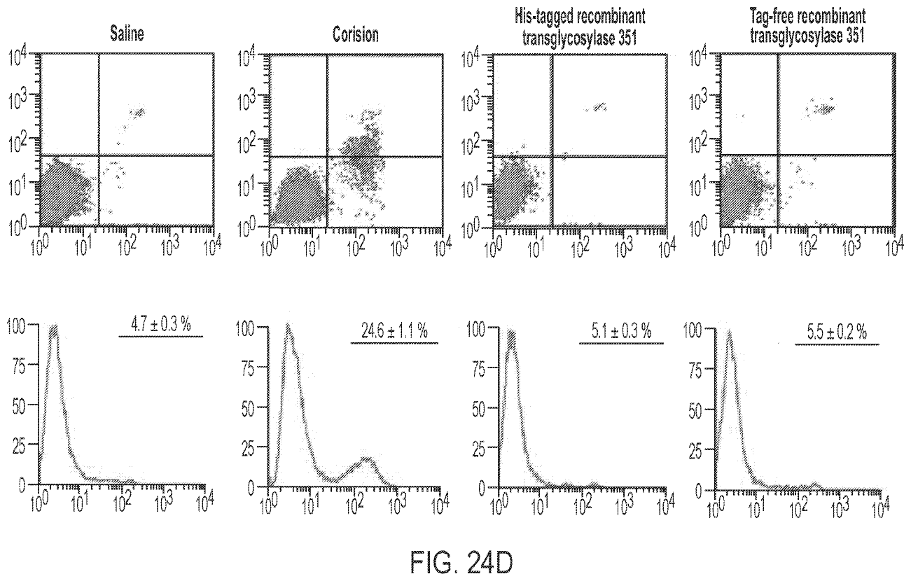

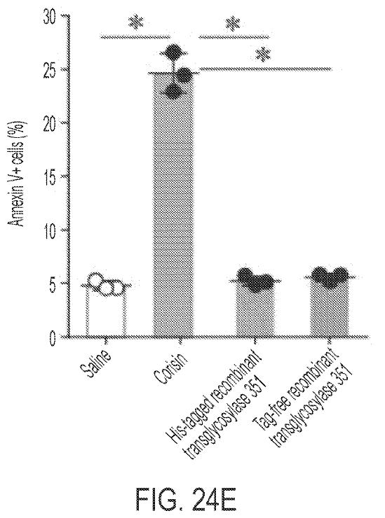

We prepared 6-Histidine-tagged (His-tagged) or Tag-free (the His-tag was cleaved) recombinant full-length transglycosylase 351, expressed in E. coli cells, to evaluate apoptotic activity on A549 cells. The unheated or heated recombinant His-tagged transglycosylase 351 (see FIGS. 24A-24B) and the Tag-free recombinant transglycosylase 351 (FIGS. 24C-24E) failed to induce apoptosis in lung epithelial cells, thereby providing evidence of the need for polypeptide processing and corisin release for biological activity.

More specifically, FIGS. 24A and 24B show a flow cytometry analysis of A549 alveolar epithelial cells after culturing for 48 h in DMEM medium containing 10 μg/ml corisin, unheated or heated His-tagged recombinant transglycosylase. Each treatment group had n=3. Bars indicate the means ±S.D. Statistical analysis was performed by ANOVA with Tukey's test. *p<0.001.

FIG. 24C shows the result of a gel electrophoresis using sodium dodecyl sulfate polyacrylamide gel (10-20%) and silver-staining of thrombin-treated or thrombin-untreated His-tagged recombinant transglycosylase 351 from Staphylococcus nepalensis strain CNDG. Representative microphotographs out of two experiments with similar results are shown.

FIGS. 24D and 24E show a flow cytometry analysis of A549 alveolar epithelial cells after culturing for 48 h in DMEM medium containing 10 μg/ml corisin, His-tagged or Tag-free recombinant transglycosylase. Each treatment group had n=3. Bars indicate the means ±S.D. Statistical analysis was performed by ANOVA with Tukey's test. *p<0.001.

Corisin Exacerbates Pulmonary Fibrosis in hTGFβ1 TG mice

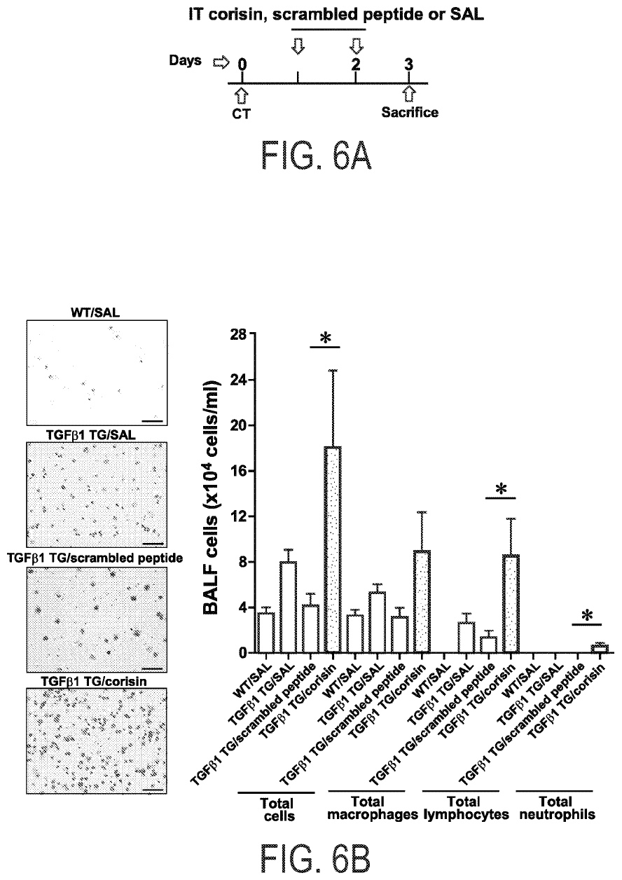

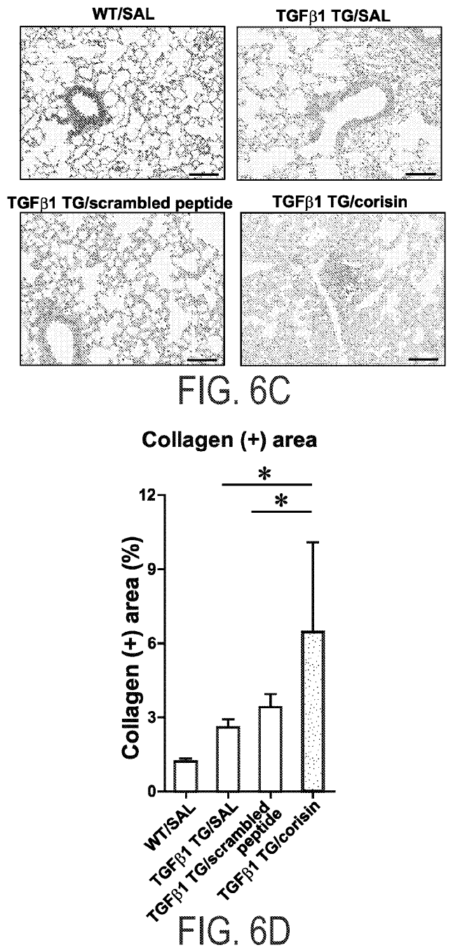

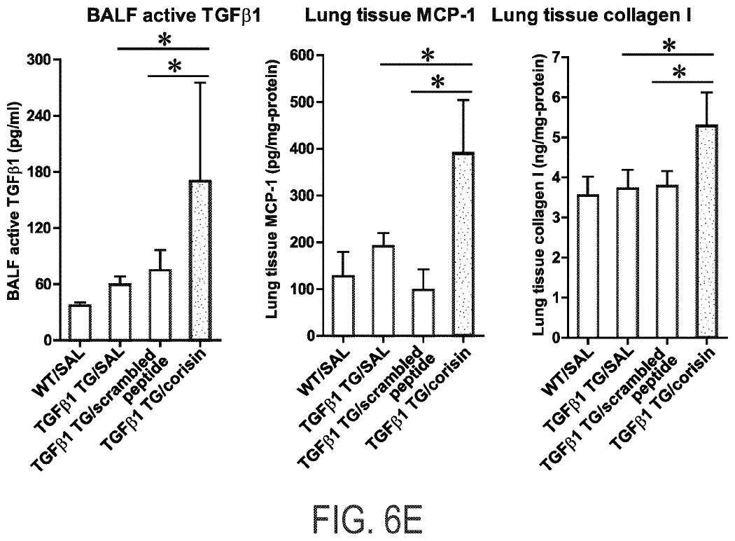

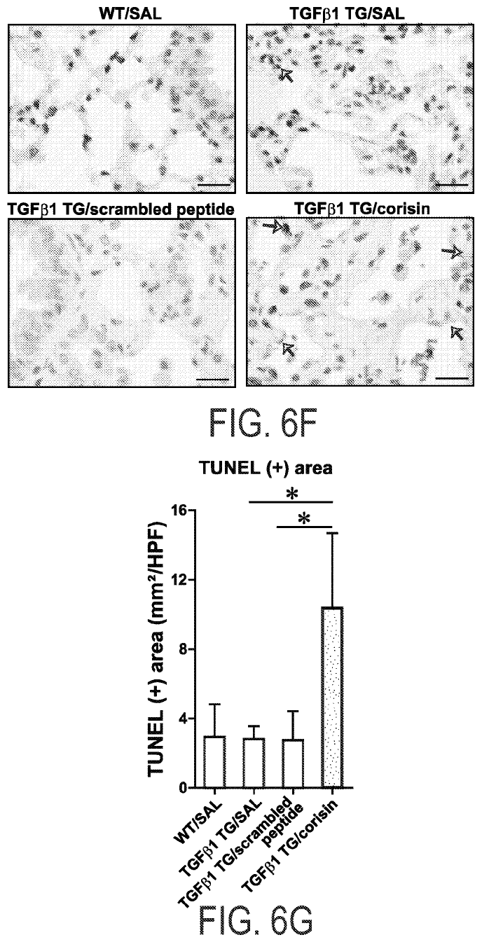

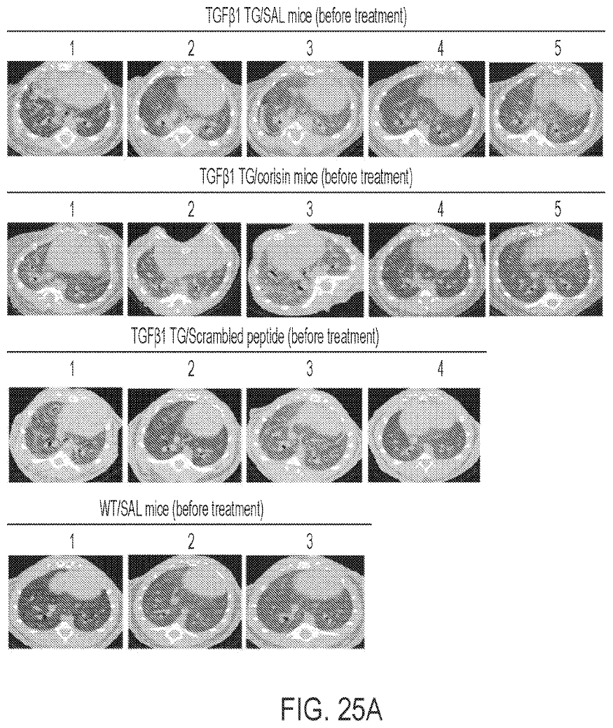

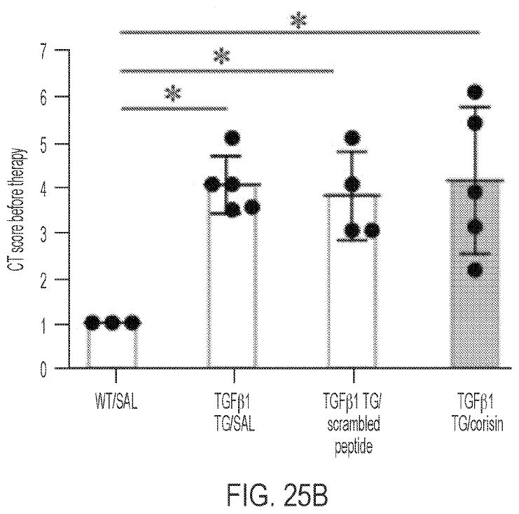

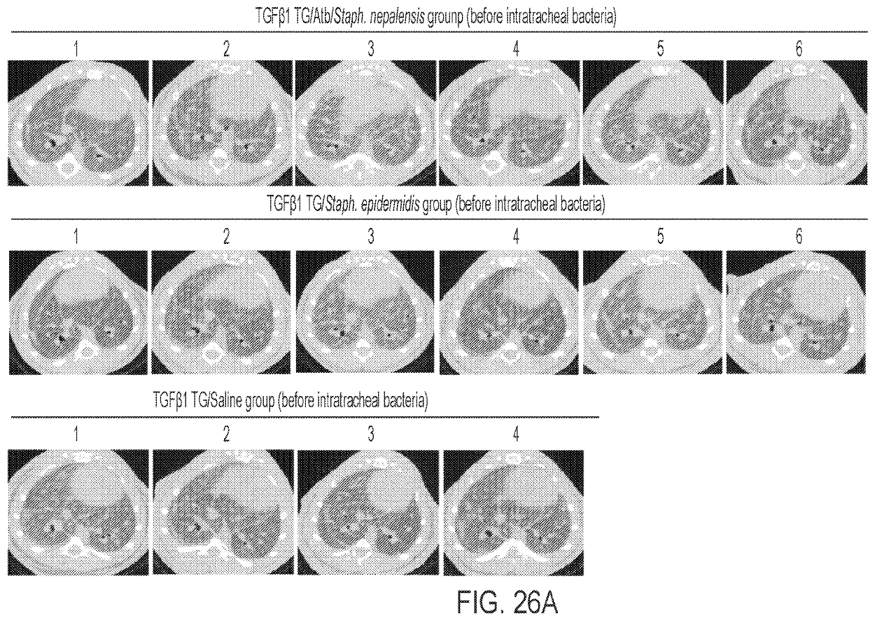

To investigate whether corisin can exacerbate the lung fibrotic disease in vivo, we separated TGFβ1 TG mice into three groups with matched level of lung fibrosis (see FIGS. 25A and 25B) and treated them with saline, scrambled peptide or corisin by the intratracheal route once daily for two days before euthanasia on day 3 (see FIG. 6A).

TGFβ1 TG mice receiving corisin exhibited significantly increased infiltration of macrophages, lymphocytes and neutrophils, increased collagen deposition and concentration of inflammatory cytokines and chemokines, and enhanced apoptosis of epithelial cells in the lungs compared to control mice (see FIGS. 6B-6G), thereby demonstrating the detrimental effect of the pro-apoptotic activity of corisin in vivo.

More specifically, FIGS. 25A and 25B respectively show computed tomography (CT) images and CT fibrosis scoring of WT mice (n=3) and TGFβ1 TG mice before treatment with saline (n=5), scrambled peptide (n=4) or corisin (n=5) that were performed as described in the methods below. Bars indicate the means ±S.D. Statistical analysis was performed by ANOVA with Tukey's test. *p<0.05. There was no statistical difference (p=0.9) between TGFβ1 TG/SAL, TGFβ1 TG/scrambled peptide, and TGFβ1 TG/corisin groups.

S. nepalensis Instillation Exacerbates Pulmonary Fibrosis

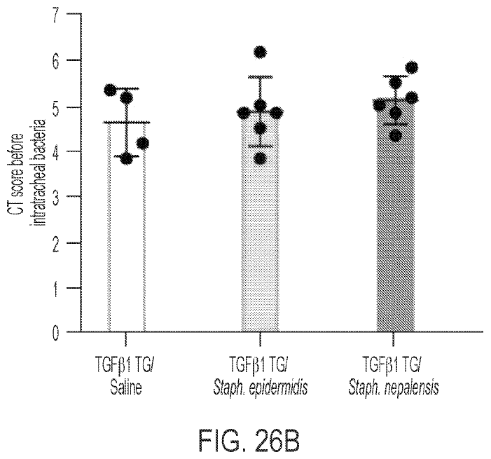

We evaluated in vivo whether bacteria that express transglycosylases containing the corisin sequence also exacerbate lung fibrosis. To this end, we intratracheally administered Staphylococcus nepalensis strain CNDG, which contains the corisin sequence, or Staphylococcus epidermidis [ATCC14990], as negative control, to germ-free TGFβ1 TG mice separated in three groups with matched lung fibrosis CT scores (see FIGS. 26A and 26B).

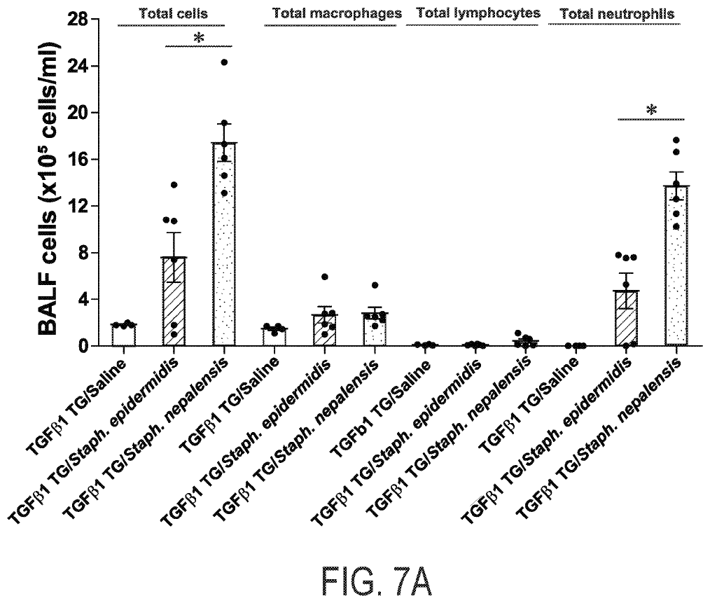

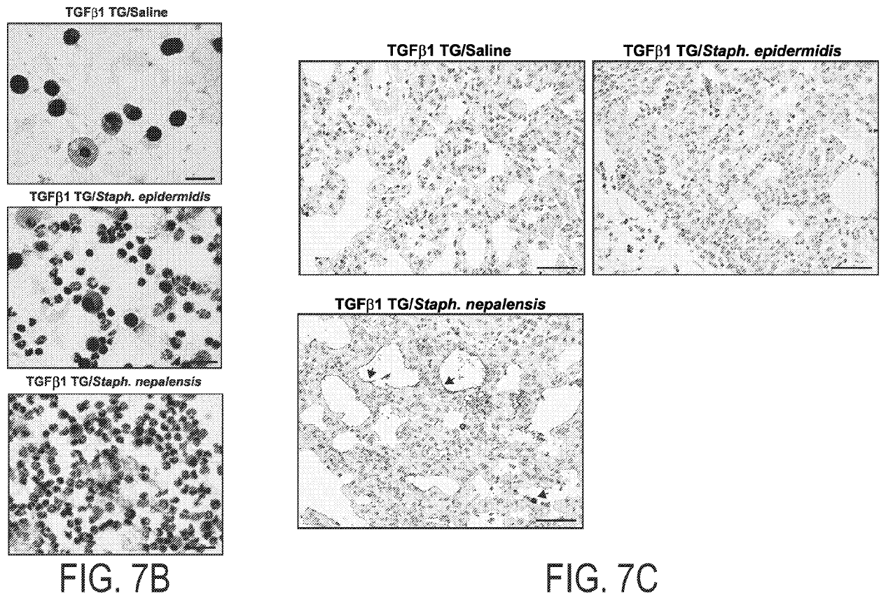

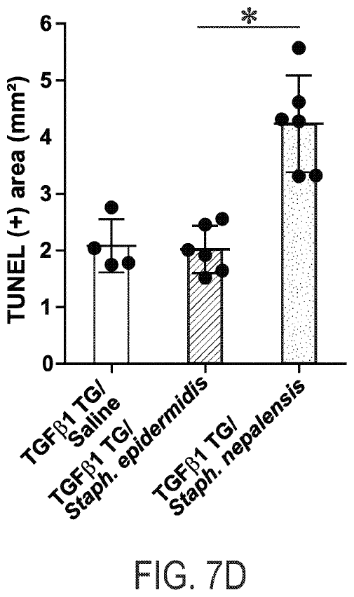

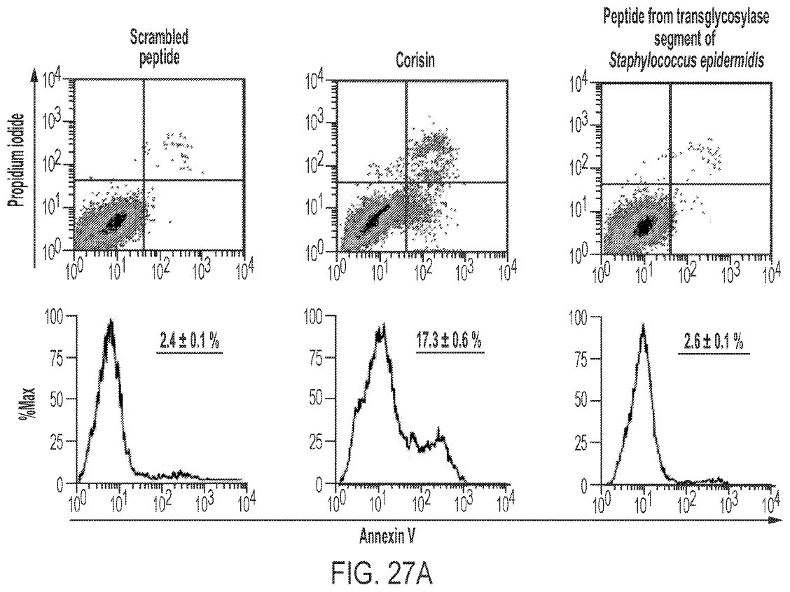

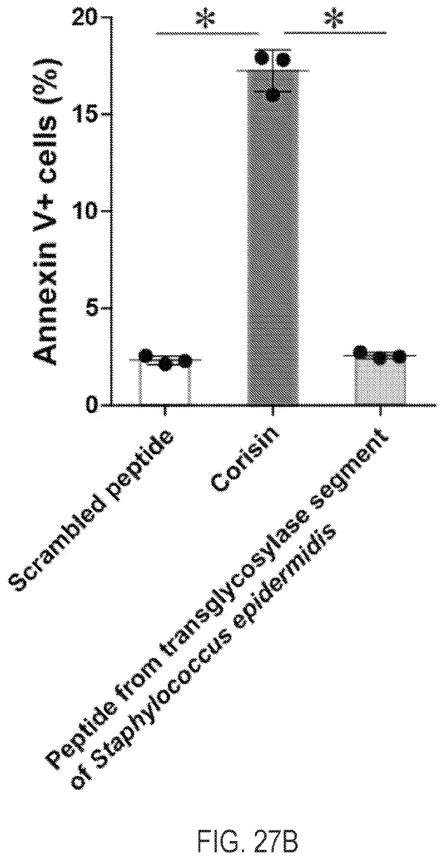

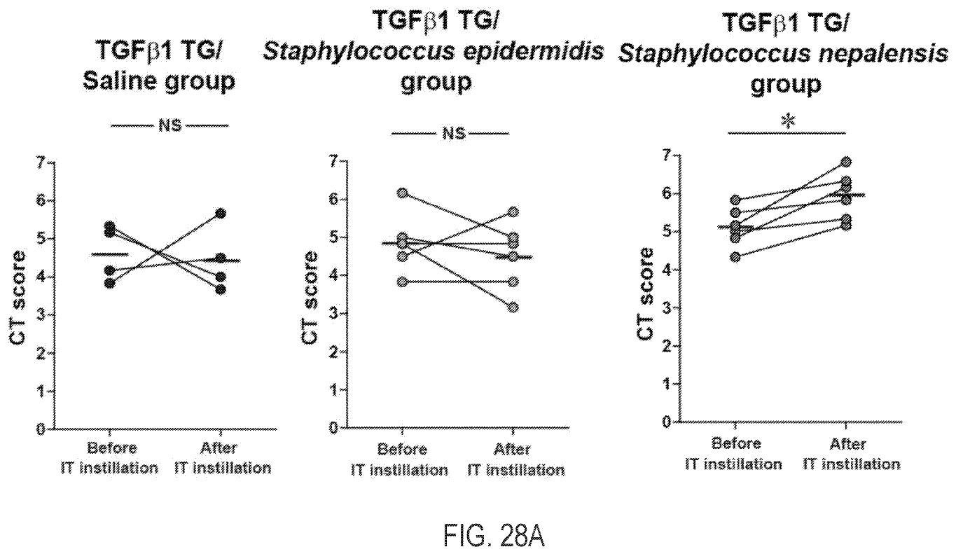

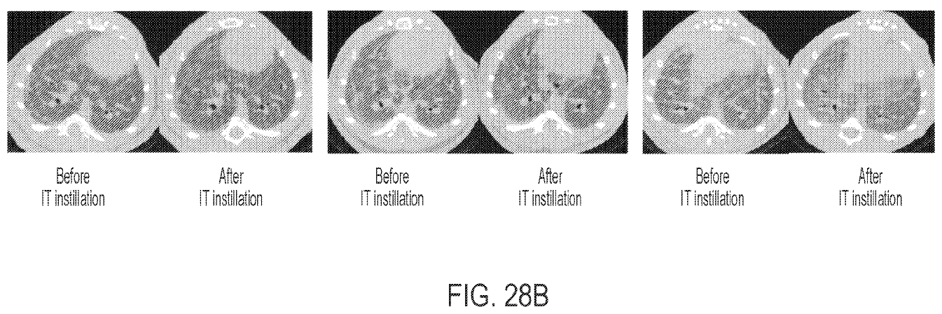

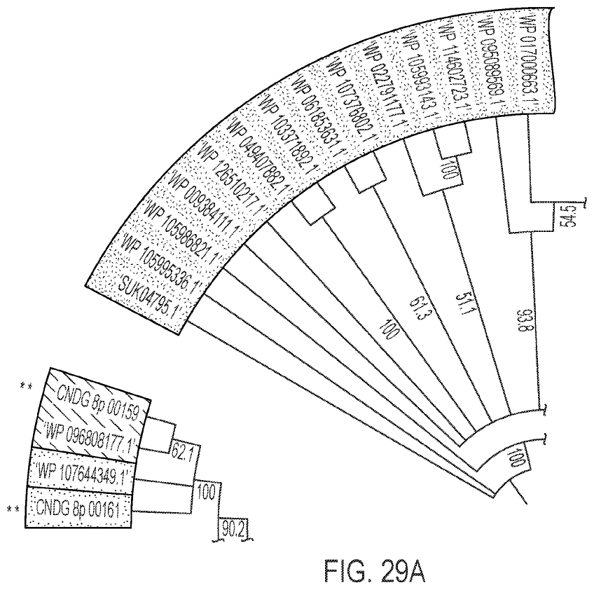

Before this in vivo experiment, we corroborated in vitro that a synthetic peptide (IIARESNGQLHARNASGAA—SEQ. ID NO.:2) corresponding to the peptide sequence at the “corisin position” of the transglycosylase from Staphylococcus epidermidis exerts (exhibits) no pro-apoptotic effect on lung epithelial cells (see FIGS. 27A and 27B). TGFβ1 mice instilled with Staphylococcus nepalensis strain CNDG showed significant worsening of lung radiological findings (see FIGS. 28A and 28B), and significantly increased neutrophil infiltration, and enhanced alveolar epithelial cell apoptosis as compared to mice receiving Staphylococcus epidermidis (see FIGS. 7A-7D), thereby further corroborating the role of the pro-apoptotic peptide in acute exacerbation of pulmonary fibrosis.

More specifically, FIGS. 26A and 26B respectively show computed tomography (CT) images and CT fibrosis scoring of TGFβ1 TG mice before intratracheal instillation of Staphylococcus nepalensis (n=6), Staphylococcus epidermidis (n=6) or saline (n=4) as further described in the methods below. Bars in FIG. 26B indicate the means ±S.D. Statistical analysis was performed by ANOVA with Tukey's test. There was no statistical difference (p=0.5) among the mouse groups.

FIGS. 27A and 27B show a flow cytometry analysis of A549 alveolar epithelial cells after culturing for 24 h in DMEM medium containing 10 μM of synthetic peptide containing the sequence of the transglycosylase segment (corisin) from Staphylococcus nepalensis strain CNDG (IVMPESSGNPNAVNPAGYR—SEQ. ID NO.:1), its scrambled peptide (NRVYNGPAASPVSEGMPIN—SEQ. ID NO.:3) or synthetic peptide of the transglycosylase segment from Staphylococcus epidermidis (ATCC14990) (IIARESNGQLHARNASGAA—SEQ. ID NO.:2). Each treatment group had n=3 (triplicates). Bars in FIG. 27B indicate the means ±S.D. Statistical analysis was performed by ANOVA with Tukey's test. *p<0.001.

FIGS. 28A and 28B respectively show computed tomography (CT) images and CT fibrosis scoring of TGFβ1 TG mice that were performed before and after intratracheal instillation of saline (n=4), Staphylococcus epidermidis (n=6) or Staphylococcus nepalensis (n=6) in germ-free TGFβ1 TG mice as described further in the methods below. Bars in FIG. 28A indicate the means ±S.D. Statistical analysis was performed by two-tailed Mann-Whitney U test. *p<0.05.

### Detection of Corisin in the Lungs of Mice and Human Patients

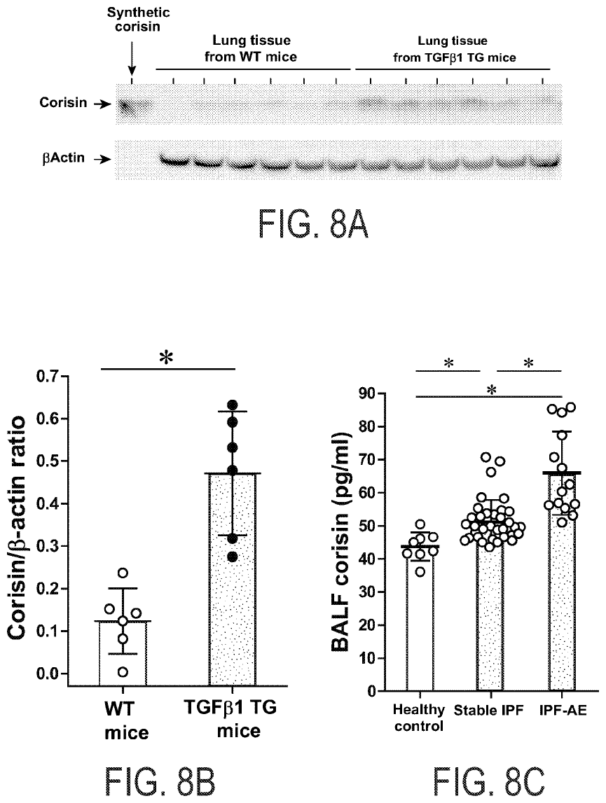

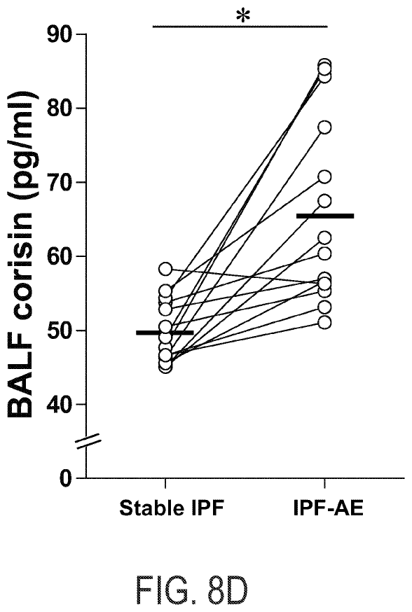

We explored the presence of corisin in WT mice without fibrosis and in TGFβ1 TG mice with and without fibrosis. We found a significantly enhanced level of corisin in TGFβ1 TG mice with lung fibrosis compared to WT mice and TGFβ1 TG mice without fibrosis (see FIGS. 8A and 8B).

To clarify the clinical relevance of this finding, we also evaluated corisin in human IPF patients. To this end, we collected bronchoalveolar lavage fluids from 34 IPF patients and 8 male healthy controls. The characteristics of the IPF patients are described in Table 3 below.

TABLE 3

| | Number of patients |
|---|---|
| Clinical parameters | and mean values |
| No of Japanese patients | 34 |
| Sex | |
| Male | 29 |
| Female | 5 |
| Age (years-old) | 71.7 ± 6.6 |
| Smoking history | |
| Current smoker | 2 |
| Ex-smoker | 25 |
| Never smoker | 7 |
| Lung function test | |
| VC (L) | 2.7 ± 0.7 |
| VC (% predicted) | 80.8 ± 17.3 |
| FVC (L) | 2.7 ± 0.7 |
| FVC (% predicted) | 83.3 ± 18.4 |
| FEV1 (L) | 2.1 ± 0.6 |
| FEV1/FVC (%) | 78.8 ± 10.9 |
| Rest SpO₂ (%) | 95.6 ± 2.2 |
| Therapy | |
| None | 32 |
| Nintedanib | 2 |

Data are the mean ± S.D. IPF, idiopathic pulmonary fibrosis; VC, vital capacity; FEV1, forced expiratory volume in one second; FVC, forced volume vital capacity; L, liters; SpO₂, arterial oxygen saturation by pulse oximetry.

The level of corisin in bronchoalveolar lavage fluid (BALF) was significantly increased in IPF patients with stable disease or with acute exacerbation compared to healthy controls (see FIGS. 8C and 8D). The BALF corisin level was also significantly elevated in IPF patients with acute exacerbation compared to patients with stable disease (see again FIGS. 8C and 8D). The difference in the level of corisin was not statistically significant (p=0.07) between males (50.6±4.9 μg/ml) and females (58.8±10.7 μg/ml). The corisin level was also not significantly correlated (r=0.1, p=0.5) with the age of the patients. These results provide evidence of the clinical relevance of corisin in IPF.

A dramatic increase of apoptotic epithelial cells occurs in the lungs of IPF patients with acute exacerbation. See NPL15 and NPL16. The results herein provide evidence that excessive release of the bacterial-derived pro-apoptotic corisin will contribute to this fatal disease complication.

### Phylogenetic Analysis Reveals Conservation of Corisin

To unveil the evolutionary relationship of transglycosylases expressed by different bacteria, we constructed a phylogenetic tree based on the amino acid sequences of six transglycosylases identified in the genome of Staphylococcus nepalensis strain CNDG and their homologs in a publicly available database (www.ncbi.nlm.nih.gov/pubmed), as will be further described below.

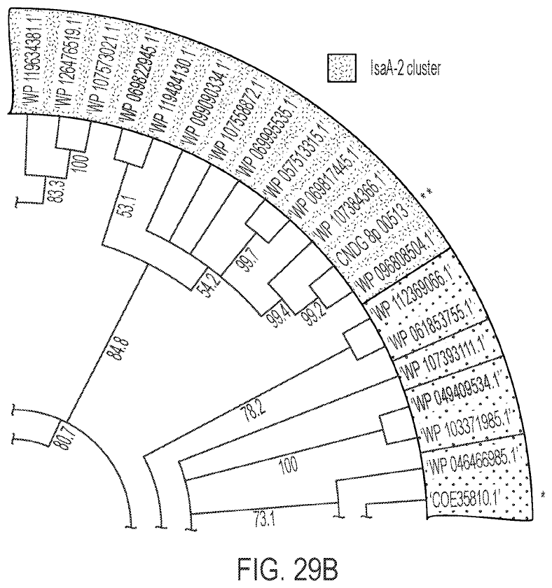

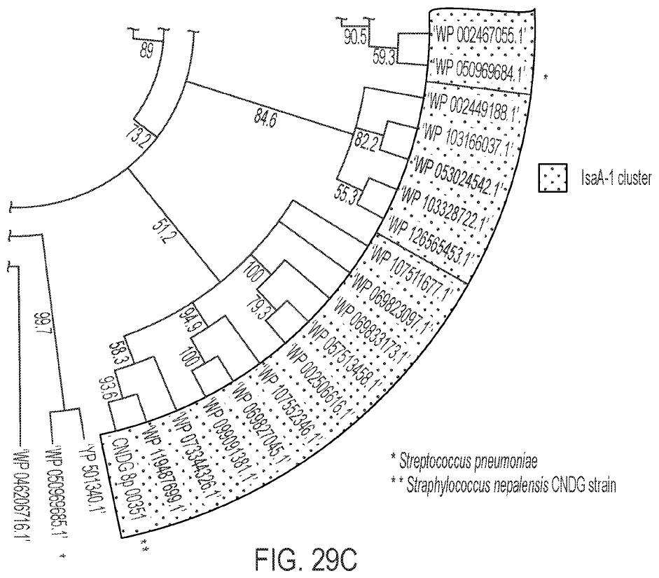

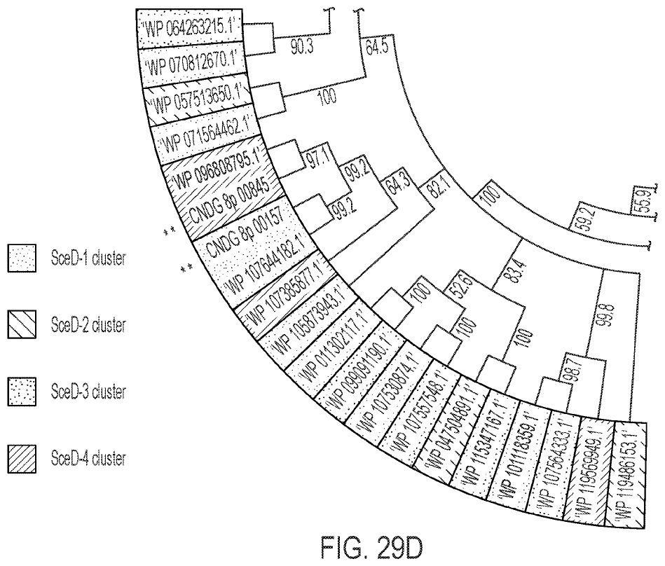

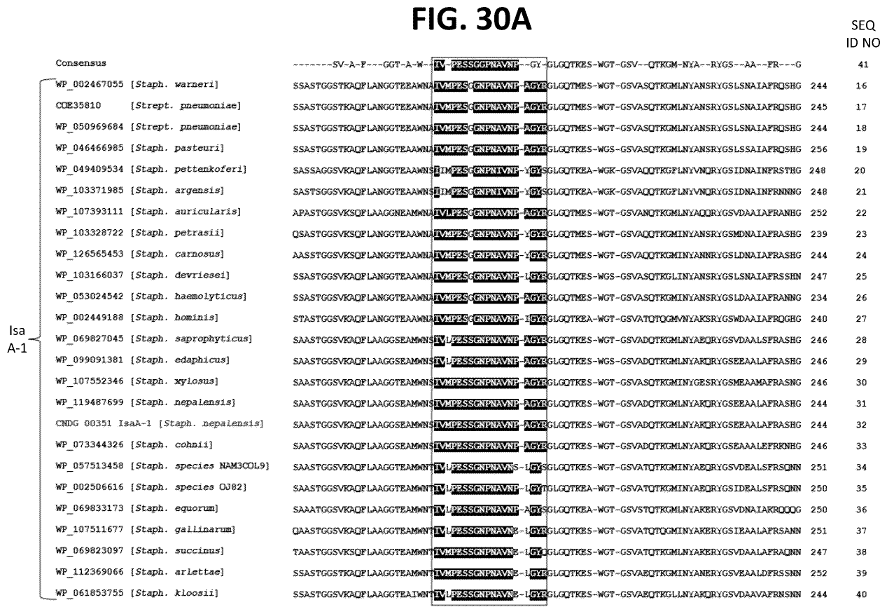

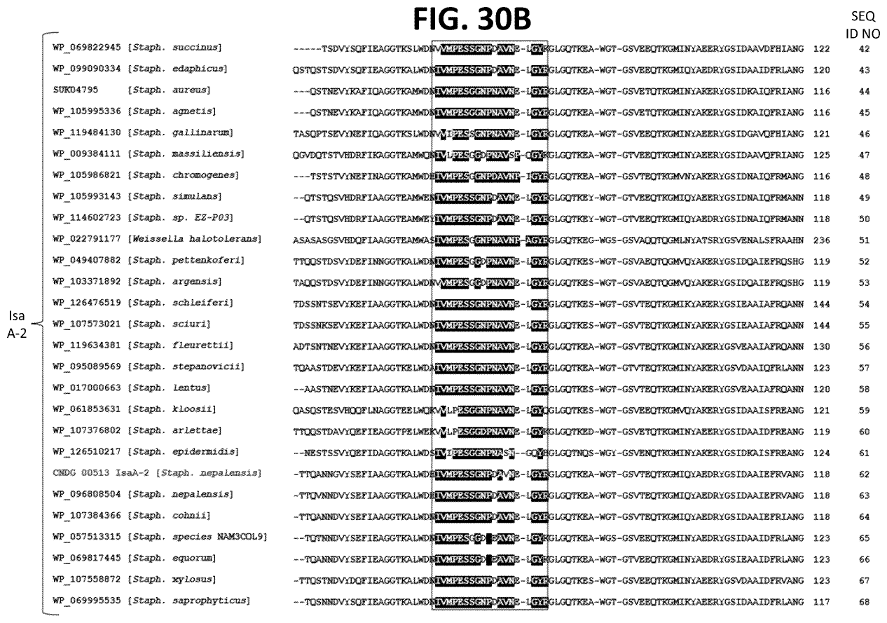

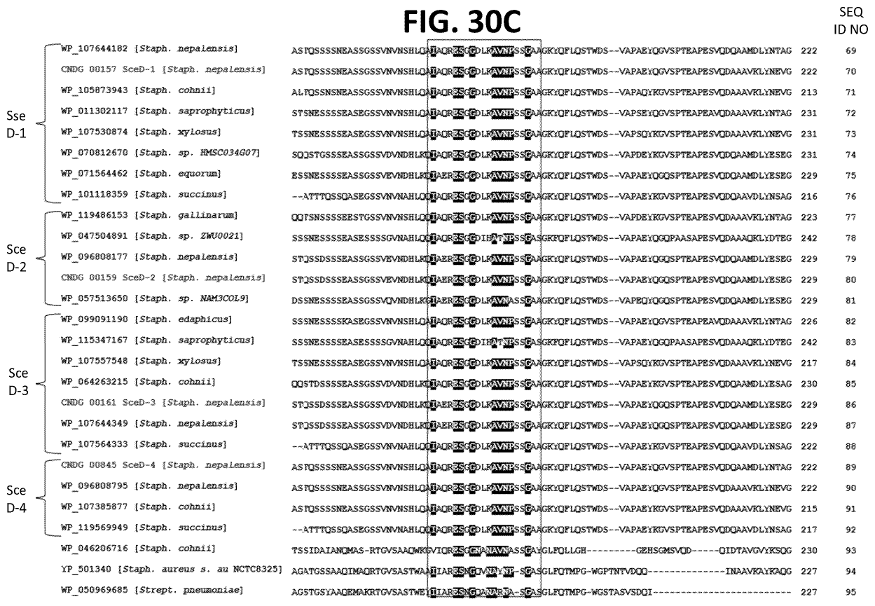

The topology of the phylogenetic tree shows that a derivative of the transglycosylases close to the ancestral sequence splits into the two IsaA clusters (IsaA-1 and IsaA-2) and from IsaA-1 related sequences, the proteins designated SceD members likely evolved (SceD-1, SceD-2, SceD-3, SceD-4) (see FIGS. 29A-29D). The multiple alignment of the IsaA and the SceD amino acid sequences revealed, in general, conservation of amino acid residues representing the pro-apoptotic corisin, and thus highlighting their functional significance (see FIGS. 30A, 30B and 30C).

The amino acid sequence identity of corisin homologous transglycosylases from Staphylococcus xylosus, Staphylococcus cohnii and Staphylococcus nepalensis was 100%.

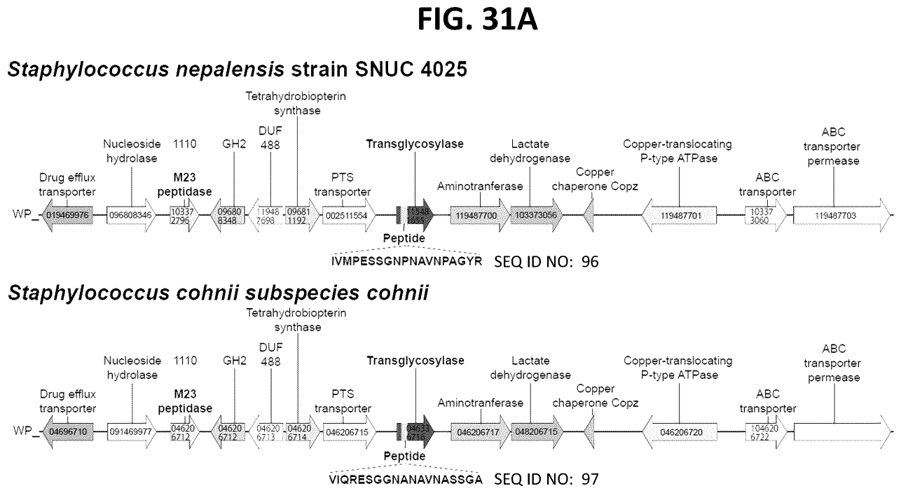

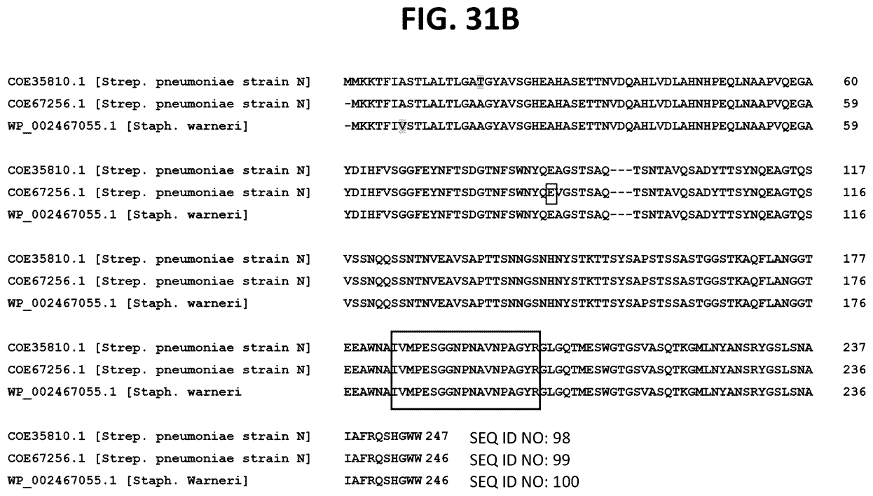

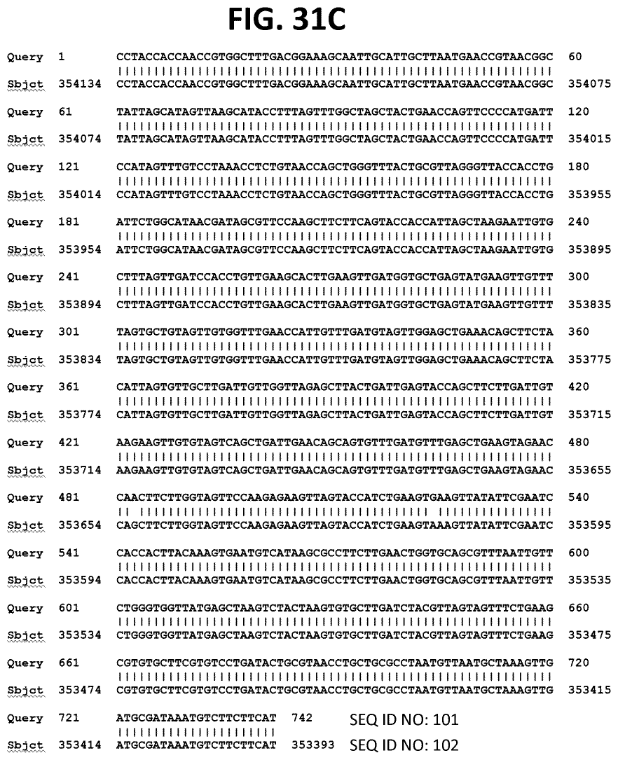

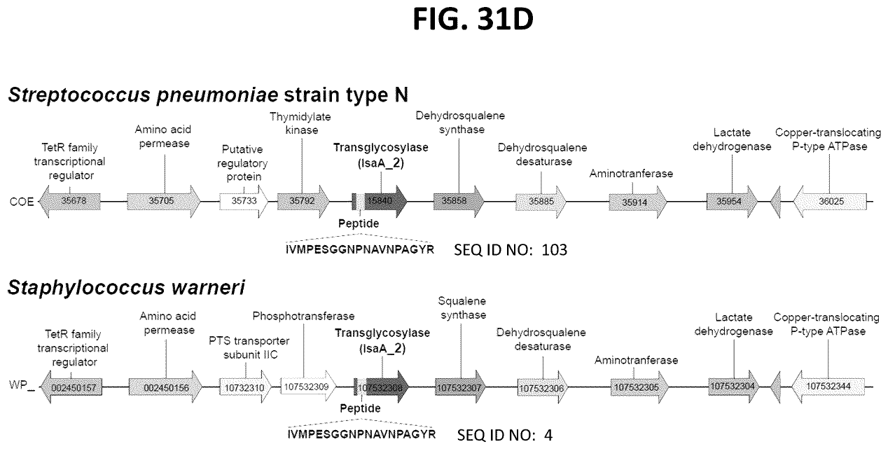

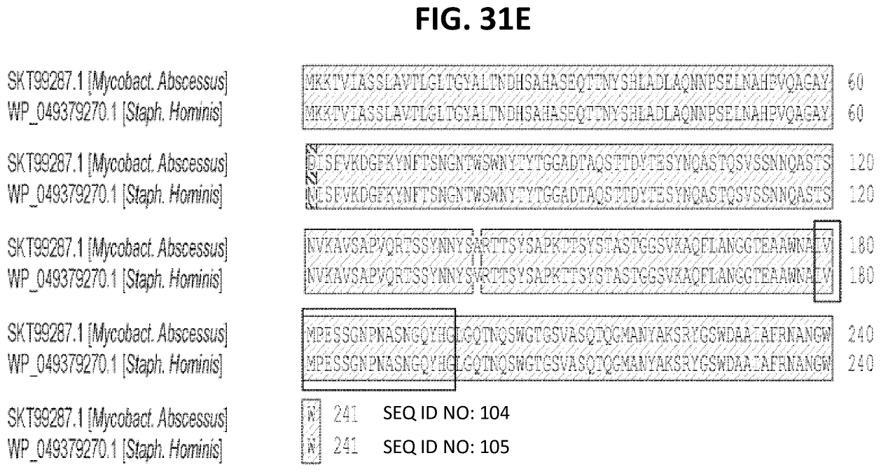

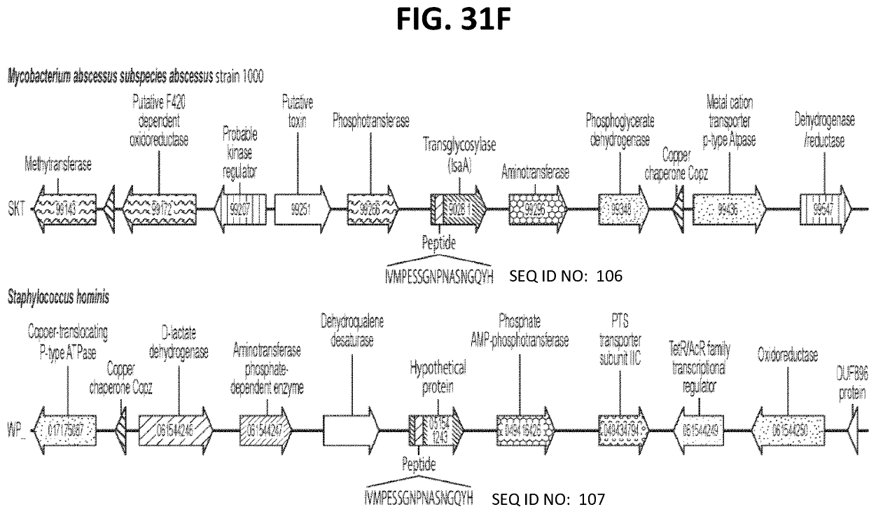

Furthermore, these Staphylococci shared more than 98% identity with the corresponding corisin regions of transglycosylases from other members of the IsaA-1 and IsaA-2 clusters, and 60% identity with the corresponding regions in members of the SceD clusters (see FIGS. 30A, 30B and 30C). The genomic context of genes clustering around the transglycosylase (synteny) tended to be conserved in Staphylococcus cohnii and Staphylococcus nepalensis (see FIG. 31A).

In particular, FIGS. 30A-30C show, for example, the following amino acid sequences that are deemed to be, or fall within the scope of the term, “corisin” in the context of the present teachings, namely:

(SEQ ID NO: 4)
IVMPESGGNPNAVNPAGYR,
(SEQ ID NO: 5)
IIMPESGGNPNIVNPYGYS,
(SEQ ID NO: 6)
IVMPESGGNPNAVNPYGYR,
(SEQ ID NO: 7)
IVLPESSGNPNAVNPAGYR,
(SEQ ID NO: 8)
IVLPESSGNPNAVNELGYR,
(SEQ ID NO. 9)
IVMPESGGNPNAVNELGYR,
(SEQ ID NO. 10)
IVMPESSGNPNAVNELGYR,
(SEQ ID NO. 11)
IVMPESSGNPDAVNELGYR,
(SEQ ID NO. 12)
IAQRESGGDLKAVNPSSGA,
and
(SEQ ID NO. 13)
IAERESGGDLKAVNPSSGA

### Horizontal Gene Transfer of the Corisin-Encoding Gene

Sequence alignment and comparative genome analysis revealed that a pathogenic strain of Streptococcus, i.e., Streptococcus pneumoniae strain N, implicated in respiratory tract disease, contains a transglycosylase (COE35810) with a peptide sequence almost identical (a single amino acid change) to corisin.

A further examination of the genome of this bacterium unveiled a second homolog (COE67256) of the corisin-containing polypeptide (FIGS. 30A, 30B and 30C).

To understand how Streptococcus pneumoniae strain N might have acquired the corisin-encoding gene, since its polypeptide sequence is highly conserved only in diverse Staphylococcus spp., we performed a search in the Genbank database and found that the polypeptide (COE35810) yields 98-100% identity with transglycosylases in different strains of Staphylococcus warneri (WP_002467055, WP_050969398, WP_126403073, and WP_107532308) (see FIGS. 31B and 31C). Despite the one or two changes in amino acids at the N-terminal region of the polypeptides, the corisin peptide sequences within these transglycosylases are invariant.

We further examined the genomic context of these genes in Streptococcus pneumoniae strain N in comparison with a Staphylococcus warneri strain, and found a clear conservation of synteny, despite some differences in annotation (see FIG. 31D).

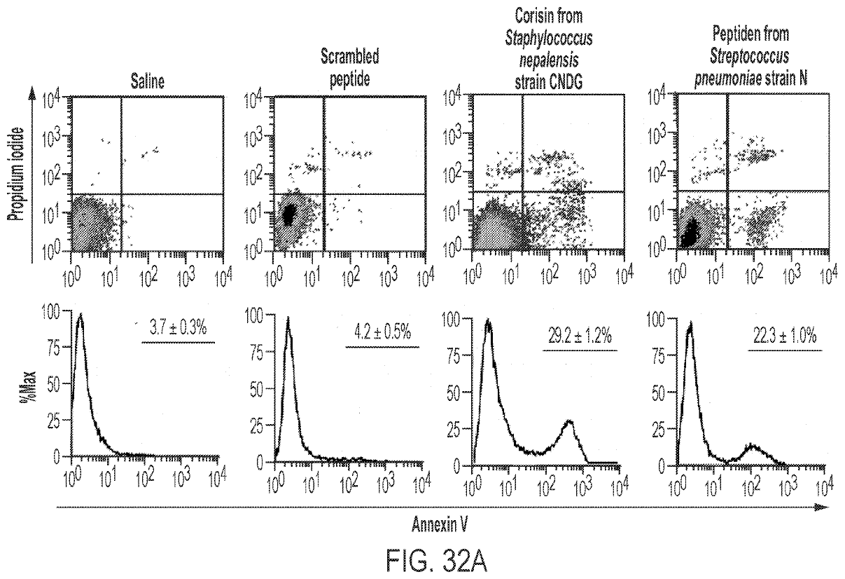

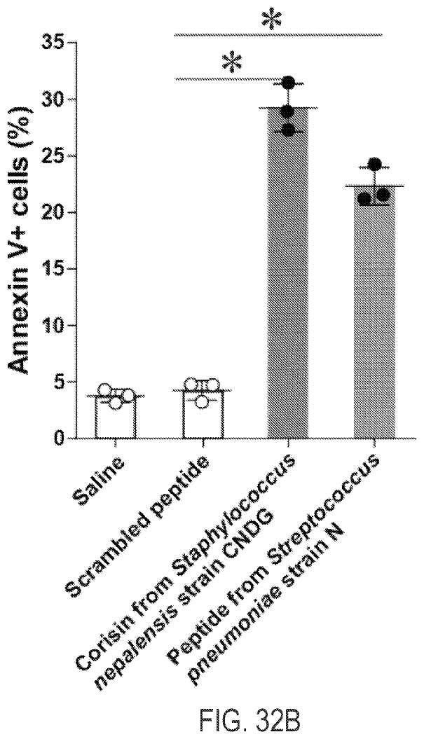

We therefore hypothesized that the transglycosylase gene and other genes linked to it in Streptococcus pneumoniae strain N were acquired from a Staphylocccus warneri strain or a related species. Significantly, strains of another pathogenic bacterium are known to inhabit the human lung. For example, Mycobacterium [Mycobacteroides] abscessus harbors (contains) a variant of the transglycosylase (SKT99287). Based on a similar analysis as was described above for Streptococcus pneumoniae strain N, we inferred that the transfer was from Staphylococcus hominis or related species (see FIGS. 31E and 31F). We then performed an experiment that confirmed that the synthetic corisin from the transglycosylase of Streptococcus pneumoniae (contains 1 amino acid change from Staphylococcus nepalensis derivative) also induces apoptosis of A549 alveolar epithelial cells (see FIGS. 30A-30C, 32A and 32B).

More particularly, FIGS. 32A and 32B show a flow cytometry analysis of A549 alveolar cells after culturing for 48 h in DMEM medium containing 5 μM of the synthetic corisin (IVMPESSGNPNAVNPAGYR) from Staphylococcus nepalensis (strain CNDG) transglycosylase 351, its scrambled peptide (NRVYNGPAASPVSEGMPIN) or the synthetic peptide (IVMPESGGNPNAVNPAGYR) from Streptococcus pneumoniae strain N transglycosylases (COE35810 and COE6725). Each group had n=3. Bars in FIG. 32B indicate the means ±S.D. Statistical analysis was performed by ANOVA with Tukey's test. *p<0.001.

From these observations, it is concluded that non-Staphylococcus organisms that have the genes encoding transglycosylases with very high homology to the Staphylococcus nepalensis transglycosylase 351 are lung-associated, thereby providing evidence of a case of horizontal gene transfer from Staphylococcus strains inhabiting the lung.

### DISCUSSION

TGFβ1 (transforming growth factor) is a pleiotropic cytokine having a pivotal role in the pathogenesis of pulmonary fibrosis owing to its potent stimulatory activity on extracellular matrix synthesis, activation, differentiation and migration of myofibroblasts, epithelial-to-mesenchymal transition, and production of pro-fibrotic factors and apoptosis of alveolar epithelial cells. See NPL17 and NPL18. The development of pulmonary fibrosis in TG mice that overexpress TGFβ1 is a proof-of-concept for the critical role of this cytokine in tissue fibrosis. See NPL11. In addition, TGFβ1 may promote exacerbation of pulmonary fibrosis by directly suppressing both the innate and adaptive immune systems leading to enhanced host susceptibility to infection. See NPL19, NPL20 and NPL21.

NPL22, NPL23 and NPL24 have shown that high salt concentration impairs host defense mechanisms by suppressing the activity of antimicrobial peptides or by altering the population of immune cells. Therefore, TGFβ1 may also indirectly affect the host immune response by favoring the accumulation of salt in the extracellular space. See NPL25 and NPL26. Abnormal extracellular storage of salt may result from TGFβ1-mediated negative regulation of the surface expression of epithelial sodium and chloride channels leading to decreased transport of Na+ and Cl− ions from the alveolar airspaces across the epithelium. See also NPL27-NPL29.

Consistent with these findings, as shown in the present disclosure, we found in lung tissue a significant increase of sodium level in TGFβ1 TG mice with lung fibrosis compared to WT mice, a significant positive correlation of sodium level with fibrotic markers and pro-fibrotic cytokines, and a significant negative correlation of sodium level with lymphocyte count and sodium and chloride channels.

A recent single-cell RNA sequencing study showing that expression of several cell membrane sodium and chloride transporters is significantly altered in alveolar epithelial cells from IPF patients, thereby suggesting that ion transmembrane trafficking is disrupted in pulmonary fibrosis and favors the accumulation of salt in this fibrotic disease. See NPL30. Sodium storage appears to require the presence of fibrotic matrix, because we found no difference in the lung sodium level between TGFβ1 TG mice without fibrosis and WT mice. In this connection, previous studies have shown that sodium is stored in extracellular spaces in an osmotically inactive form by binding to negatively charged glycosaminoglycans, which are abundant in the extracellular matrix of fibrotic tissues. See NPL31-NPL35.

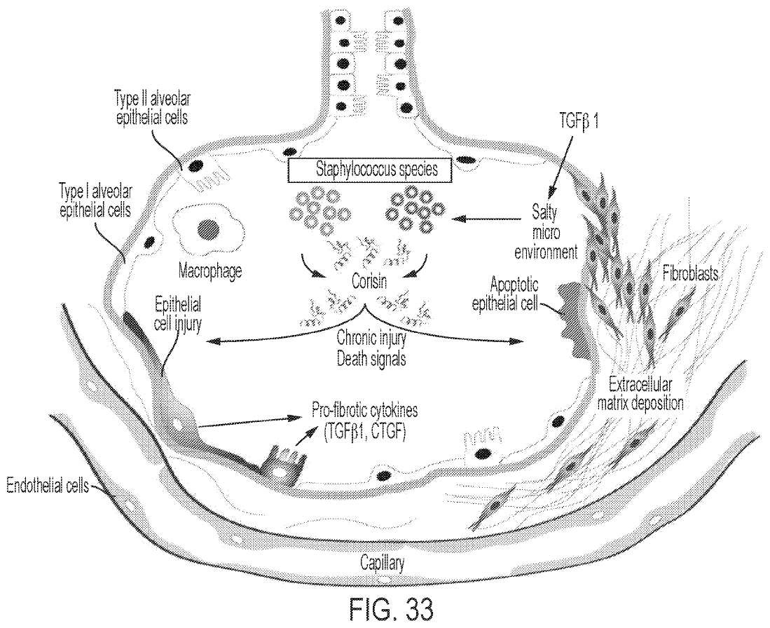

Overall, these observations suggest that the fibrotic tissue is a salty microenvironment (see model in FIG. 33) with abnormal immune and healing responses. More specifically, transforming growth factor TGFβ1 may increase the extracellular salt concentration by downregulating the cell surface expression of ion transporters, and the salty microenvironment stimulates the growth of Staphylococcus spp. that release corisin to induce apoptosis of alveolar epithelial cells. Excessive apoptosis and/or activation of epithelial cells contribute to acute exacerbation of pulmonary fibrosis. The identification of halophilic bacteria in the lungs of IPF patients by previous studies support these findings. See NPL8 and NPL9.

Acute exacerbation is a devastating complication of IPF. See NPL36. Nearly 50% of patients dying from IPF have a prior history of acute exacerbation and the life expectancy of patients with a previous acute exacerbation is only 3 to 4 months. See NPL37-NPL41.

There is currently no optimal therapy for acute exacerbation of IPF. See NPL36. An international working group in 2016 proposed to classify this complication into triggered (identified event: post-procedure, drug toxicity, infection, aspiration) or idiopathic (unidentified inciting event) acute exacerbation. Id. Recent data associating acute exacerbation with the lung microbiome and with the host immunosuppressive states, and retrospective studies showing the preventive effect of antibiotic therapy suggest the role of infection in the pathogenesis of acute exacerbation and progression of pulmonary fibrosis. See NPL7 and NPL42-NPL45. Further, a double-blind, randomized, placebo-controlled study showing improvement of symptoms and exercise capacity in progressive IPF patients treated with co-trimoxazole, and a subsequent double-blind follow-up and multicenter study showing significant reduction of mortality with better quality of life and less respiratory tract infections in IPF patients treated with co-trimoxazole also support the pathogenic role of bacteria in lung fibrosis. See NPL46 and NPL47.

NPL7 showed that bacteria of the Staphylococcus and Streptococcus genera worsen the clinical outcome of IPF patients, suggesting their implication in the disease progression and pathogenesis. Studies showing the relative abundance of Staphylococcus or Streptococcus genera in the fibrotic lung and its significant correlation with the host immune response in IPF patients further support the contribution of these bacteria genera in the pathogenesis of pulmonary fibrosis. See NPL6, NPL 42 and NPL48-NPL52. However, the precise mechanism remains unclear.

In the research that resulted in the present disclosure, we hypothesized that a salty culture medium would mimic the in vivo salty fibrotic tissue and thus would favor the growth of bacteria involved in the pathogenesis of lung fibrosis. We detected growth of bacteria of the genus Staphylococcus in the hypersaline media inoculated with fibrotic tissues from hTGFβ1 TG mice with advanced fibrosis, and the whole genome sequence of a pure bacterial culture revealed that it corresponds to Staphylococcus nepalensis that we categorized as “strain CNDG”. The culture supernatant of this bacterium induced apoptosis of alveolar epithelial cells, and subsequent chromatography, mass spectrometry and gene sequence analysis showed that apoptosis was induced by a peptide that we called “corisin” that corresponds to a segment of transglycosylase 351 from Staphylococcus nepalensis strain CNDG. The higher apoptotic activity of supernatants from bacteria cultured under high-salt conditions may be due to salt-dependent stimulation of bacteria growth or increased bacterial expression of the corisin-containing transglycosylase, which is a related protein that has been reported to be enhanced in expression in Staphylococcus aureus under similar conditions. See NPL53.

In additional experiments, we detected the peptide in the lung from hTGFβ1 TG mice with progressive lung fibrosis and from patients with IPF and found that intratracheal instillation of synthetic corisin or Staphylococcus nepalensis strain CNDG induces acute exacerbation of pulmonary fibrosis in association with extensive apoptosis of alveolar epithelia cells (see the model in FIG. 33). Accelerated apoptosis of alveolar epithelial cells plays a central role in the pathogenesis of acute exacerbation in pulmonary fibrosis. See NPL16 and NPL54. Therefore, based on these observations, corisin emerges as a strong candidate in the microbial factors that appears to trigger acute exacerbation in patients with idiopathic pulmonary fibrosis.

We found that the sequence of corisin has high homology with a region in a membrane-bound lytic transglycosylase. Lytic transglycosylases are bacterial enzymes reported to cleave the peptidoglycan component of the bacterial cell wall (see NPL55) and further perform other essential cellular functions, such as cell-wall synthesis, remodeling, resistance to antibiotics, insertion of secretion systems, flagellar assembly, release of virulence factors, sporulation and germination (Id.). Transglycosylases are ubiquitous in bacteria and an individual species may produce multiple transglycosylases with functional redundancy, to compensate in case of loss or inactivation of any member. See NPL56 and NPL57.

In the results described herein, the complete genome sequence showed that Staphylococcus nepalensis strain CNDG produces six transglycosylases, of which the transglycosylase 351, a member of the IsaA-1 cluster, harbors (contains) the corisin sequence. The full-length transglycosylase 351 did not induce apoptosis of lung epithelial cells, thereby providing evidence that the corisin peptide is active only after being released from the full-length protein. Although the mechanism of this peptide shedding is unknown, the genomic context of the Staphylococcus nepalensis CNDG strain showing the presence of peptidases surrounding the transglycosylase 351 provides evidence that they may be involved in the release of the deadly peptide.

We found that, in addition to Staphylococcus nepalensis strain CNDG, sequences similar to corisin are highly conserved in several transglycosylases from other Staphylococcus species and some members of the microbial community that inhabit the normal or fibrotic lungs, including strains of Streptococcus pneumoniae and Mycobacterium abscessus. See NPL51 and NPL58-60. This observation provides evidence that a broad range of bacteria may be the source of corisin in pulmonary fibrosis.

Although the present disclosure is believed to be a first report on the pathogenicity of a peptide derived from an IsaA homolog in a strain of Staphylococcus, it is noted that homologous proteins (i.e., IsaA and SceD) have been reported in Staphylococcus aureus to be involved in virulence. See NPL53. The Staphylococcus aureus IsaA in NPL53 corresponds to YP_501340 in the alignment shown in FIGS. 30A, 30B and 30C, while the SceD, in the same report, has a variant of corisin similar to those in the SceD-1 to SceD-4 polypeptides (Id). Thus, although relevant, the characterized transglycosylases in Staphylococcus aureus are quite different from the Staphylococcus nepalensis transglycosylase characterized in the present study. It is of note, however, that Staphylococcus aureus has an uncharacterized IsaA transglycosylase with a highly conserved corisin sequence (FIGS. 29A-29D, IsaA-2, SUK04795.1), which may suggest that a similar mechanism as the corisin processing described in the present disclosure exists in Staphylococcus aureus.

Streptococcus pneumoniae and Staphylococcus species also frequently cause severe pulmonary infections with high in-hospital mortality rate in IPF patients. See NPL20, NPL58 and NPL61. Given the growing evidence that alveolar cell apoptosis plays a central role in the pathogenesis and exacerbation of IPF (see NPL62), it is reasonable to postulate that shedding of deadly peptides constitutes an important contribution to the loss of functional lung alveolar cells and to the poor clinical outcome in patients with complications of microbial infection.

Another mechanism that may further contribute to bacterial virulence and invasiveness is horizontal transfer of bacterial genes. See NPL63. Here we found that strains of Streptococcus pneumoniae, Mycobacterium [Mycobacteroides] abscessus and several Staphylococcus species shared highly similar genome context (synteny) and sequence homology of transglycosylases containing the corisin sequence, thereby providing evidence of the involvement of horizontal gene transfer in the acquisition of this virulence factor. Staphylococcus and Streptococcus genera are common members of the human microbiota. See NPL64. Therefore, if determined that the corisin related peptides identified in the present study have similar apoptotic impact on human cells from other sites or organs, such as the kidney and liver, our view of infections by these bacteria will require re-assessment.

In light of the increasing evidence indicating the participation of the lung microbial population in the pathogenesis of IPF, the identification of corisin as a disease exacerbator substantiates the role of apoptosis in fibrotic diseases, provides a novel diagnostic marker and therapeutic target in IPF, and opens a new avenue for investigating the role of microbiomes in organ fibrosis.

### METHODS

### Reagents

The human lung epithelial cell line A549 and hypersaline media (ATCC media 1097, 2168) were obtained from the American Type Culture Collection (Manassas, VA), Dulbecco's Modified Eagle Medium (DMEM) were obtained from Sigma-Aldrich (Saint Louis, MO) and fetal bovine serum (FBS) were obtained from Bio Whittaker (Walkersville, MD). L-glutamine, penicillin and streptomycin were obtained from Invitrogen (Carlsbad, CA). Normal human bronchial epithelial (NHBE) cells were obtained from Clonetics (Walkersville, MD). Synthetic peptides were prepared and provided by Peptide Institute Incorporation (Osaka, Japan) and by ThermoFisher Scientific (Waltham, MA, USA).

### Subjects

The study described herein comprised 34 Japanese patients with stable idiopathic pulmonary fibrosis (IPF; mean age: 71.7-6.6 years-old, males: 29, females: 5) and eight healthy Japanese male volunteers (38.3±6.1 years old). Table 3 above describes the characteristics of the patients. Diagnosis of idiopathic pulmonary fibrosis was done following accepted international criteria according to NPL65 and NPL66. Bronchoscopy study was performed following guidelines of the American Thoracic Society and bronchoalveolar lavage fluid (BALF) samples were collected from all 34 IPF patients and 8 healthy volunteers. See NPL65. BALF samples during acute exacerbation of the disease were available in 14 out of the 34 participant IPF patients. Aliquots of unprocessed bronchoalveolar lavage fluid (BALF) collected into sterile tubes were stored at −80° C. until analysis.

### Animals

We used transgenic (TG) mice in a C57BL/6J background with lung-specific overexpression of the latent form of human TGFβ1 that have been previously characterized. See NPL8 and NPL11. These TGFβ1 TG mice spontaneously develop pulmonary fibrosis from 10-weeks of age, and showed similarity to the disease in humans. Id. C57BL/6J wild-type (WT) mice were used as controls. In some of the experiments, TGFβ1 TG mice without lung fibrosis were used as controls; however, the number of mice born with the human TGFβ1 transgene positive but with no phenotype (lung fibrosis) is extremely scarce or rare and thus it was very difficult to include them in all experiments. All mice were maintained in a specific pathogen-free environment under a 12-h light/dark cycle in the facility for experimental animals of Mie University. Genotyping of TG mice were carried out using standard PCR analysis, DNA isolated from the tail of mice and primer pairs (Supplementary Table 5) as described in NPL11.

### Computed Tomography (CT)

We performed radiological evaluation of the chest of the mice using a micro-CT (Latheta LCT-200, Hitachi Aloka Medical, Tokyo, Japan). Mice received isoflurane inhalation as anesthesia and were placed in a prone position for data acquisition in accordance with NPL67. Six specialists in respiratory diseases blinded to the treatment groups scored the chest CT findings based on the following criteria: score 1, normal lung findings; 2, intermediate findings; 3, slight lung fibrosis; 4, intermediate findings; 5, moderate lung fibrosis; 6, intermediate findings; and 7, advanced lung fibrosis (FIG. 9A). See NPL67. We used the Ashcroft lung fibrosis score and the hydroxyproline content of the lungs to validate the CT findings (FIG. 9B).

### Evaluation of Pulmonary Fibrosis in Mice

Under profound anesthesia, we collected bronchoalveolar lavage fluid for biochemical analysis and cell counting. Briefly, bronchoalveolar lavage fluid was performed by cannulating the trachea with a 20-gauge needle and infusing saline solutions into the lungs in accordance with NPL68. The samples were centrifuged and the supernatants were stored at −80° C. until analysis. The cell pellets were re-suspended in physiological saline solution and the number of cells was counted. A nucleocounter from ChemoMetec (Allerød, Denmark) was used for cell counting and the cells were stained with May-Grünwald-Giemsa (Merck, Darmstadt, Germany) to count differential cells. Mice were sacrificed by anesthesia overdose, and the lungs were resected to fix in formalin, embedded in paraffin and prepared for hematoxylin and eosin staining. The severity of lung fibrosis was quantitated based on the Ashcroft criteria. See NPL67. The level of TGFβ1 was measured using a commercial enzyme immunoassay kit from BD Biosciences Pharmingen (San Diego, CA).

### Ethical Statement

All subjects participating in the clinical investigation provided written informed consent and the study protocol was approved by the Ethical Committees for Clinical Investigation of Mie University (approval No: H2019064, date: 25 Apr. 2019), Matsusaka Municipal Hospital (approval date: 11 Jun. 2014), and Chuo Medical Center (approval No 2014-6, date: 2 Aug. 2014) and conducted following the Principles of the Declaration of Helsinki. The Recombinant DNA Experiment Safety Committee (approval No: 1-614 (henkol); date: 2013 15 Dec.; approval No: 1-708, date: 13 Feb. 2019) and the Committee for Animal Investigation of Mie University approved the experimental protocols (approval No: 25-20-hen1-sai1, date: 23 Jul. 2015; approval No: 29-23, date: 15 Jan. 2019) and all procedures were performed in accordance with internationally approved principles of laboratory animal care published by the U.S. National Institute of Health.

### Lung Sampling for In Vitro Culture

Under sterile conditions, we excised the left and right lungs after euthanasia of mice by intraperitoneal injection of an overdose of pentobarbital and placed the tissue into sterile tubes and immediately stored them at −80° C. until use.

### Measurement of Lung Tissue Na+

We removed the lungs from TGFβ1 mice with or without lung fibrosis and from WT mice. The samples were sent to Shimadzu Techno-Research, Incorporation (Kyoto, Japan) for the measurement of tissue sodium content by using microwave analysis/inductively coupled plasma mass spectrometry (ICP-MS), the microwave ashing system ETHOS-TC (Milestone General) and the ICP-MS system 7700x (Agilent Technologies, Santa Clara, CA). See NPL69 and NPL70. The results are shown in FIG. 1C.

### Evaluation of Lung Tissue Immune Cells

To isolate lung immune cells, after mouse sacrifice by anesthesia overdose, we incised and minced the lung tissue with scissors into 2-3 mm pieces, incubated in 0.5 mg/ml collagenase solution for 30 min at 37° C., and then filtered through a stainless steel mesh. Lung cells were separated and purified using isotonic 33% Percoll (Sigma-Aldrich, St. Louis, MO) solution. We then detected the lung immune cells by flow cytometry using the antibodies described in Table 4 below.

TABLE 4

| Target | Label | Clone | Source | Isotype | Company |
|---|---|---|---|---|---|
| Mouse Ly-6G/Ly-6C | FITC | RB6-8C5 | rat | IgG2bκ | BioLegend, Inc. (San Diego, CA) |
| Mouse F4/80 | PE | CIA3-1 | rat | IgG2bκ | BioLegend, Inc. (San Diego, CA) |
| Mouse CD11c | PE/Cy5 | N418 | hamster | IgG | BioLegend, Inc. (San Diego, CA) |
| Mouse CD3s | FITC | 145-2C11 | hamster | IgG | BioLegend, Inc. (San Diego, CA) |
| Mouse CD45R/8220 | PE/Cy5 | RA3-6B2 | rat | IgG2aκ | BioLegend, Inc. (San Diego, CA) |
| Anti-mouse CD25 | FITC | PC61 | rat | IgG1λ | BioLegend, Inc. (San Diego, CA) |
| Mouse CD8a | PE | 53-6.7 | rat | IgG2aκ | BioLegend, Inc. (San Diego, CA) |
| mouse CD4 | PE/Cy5 | GK1.5 | rat | IgG2bκ | BioLegend, Inc. (San Diego, CA) |
| mouse NK1.1 | PE | PK136 | mouse | IgG2aκ | BioLegend, Inc. (San Diego, CA) |
| Annexin V | FITC | — | — | — | BD Pharmingen (San Diego, CA) |

FITC, fluorescein isothiocyanate; PE, phycoerythrin.

### Evaluating the Effect of the Pro-Apoptotic Corisin in Mice

Three groups of TGFβ1 TG mice (each n=5 or n=4) with matched grade (level) of lung fibrosis as assessed by CT score underwent intratracheal instillation of corisin or scrambled peptide or 0.9% NaCl solution on days 1 and 2 and sacrificed on day 3 to evaluate changes in lung inflammation and fibrosis. WT mice (n=3) without lung fibrosis treated with 0.9% NaCl solution were used as controls.

Intratracheal Instillation of Staphylococcus nepalensis

We administered by oral gavage 200 μl of a solution containing a cocktail of antibiotics including vancomycin (0.5 mg/ml), neomycin (1 mg/ml), ampicillin (1 mg/ml), metronidazole (1 mg/ml) and gentamycin (1 mg/ml) once a day for 4 days to three groups of TGFβ1 TG mice. All mice had a matched grade of lung fibrosis as assessed by CT score. On the 5th day, one group of mice received intra-tracheal instillation of 1×10⁸ colony forming units (75 μl) of Staphylococcus nepalensis strain CNDG or Staphylococcus epidermidis ATCC14990 and sacrificed after 2 days. Germ-free TGFβ1 TG mice treated with 0.9% NaCl solution were used as controls.

### Bacteria Isolation, Culturing, and Spent Medium Preparation

Lungs from TGFβ1 TG mice with lung fibrosis and from WT mice were used for in vitro microbial culture. The lung tissue specimens were washed with PBS and inoculated into ATCC medium 1097 (8% NaCl) and cultured at 37° C. with shaking at 220 rpm until growth was visible. Bacterial colonies were isolated by plating the liquid medium-cultured organisms on an ATCC medium 1097 agar plates. Each single colony was inoculated into liquid ATCC medium 1097 (8% NaCl) and cultured at 37° C. at 220 rpm for 24 h. The cultures were centrifuged for 5 min at 4,000 rpm at 4° C. to pellet the cells, and the resulting supernatant was filtered through a MILLEXGP filter unit (0.22 um, Millipore) to remove any remaining cells and used as the spent bacterial medium.

### Phase-Contrast Microscopy

We harvested bacterial cells from a single colony in exponential phase growth, immersed in a fixative overnight at 4° C. and collected microphotographs using phase contrast microscopy (Frederick Seitz Materials Research Lab, UIUC) in accordance with NPL71.

### Genomic DNA Sequencing and Genome Annotation

Genome sequencing was carried out with a combination of Oxford Nanopore Sequencing and Illumina Miseq nano sequencing that produced 6.3 Gbases and 1.6 million (2×250) nucleotides with perfect Qscores. Briefly, genomic DNA from the bacterial strain (400 ng) was converted into a Nanopore library with the Rapid Barcoding library kit SQK-RAD004. The library was sequenced on a SpotON R9.4.1 FLO-MIN106 flowcell for 48 h on a GridION sequencer. Base-calling was performed with Guppy 1.4.3, and demultiplexing was done with Porechops 0.2.3. The majority of the reads were 6 kb to 30 kb in length, although reads as long as 94 kb were also obtained. The Illumina Miseq sequencing was carried out by preparing shotgun genomic libraries with the Hyper Library construction kit from Kapa Biosystems (Roche). The library was quantitated by qPCR and sequenced on one MiSeq Nano flowcell for 251 cycles from each end of the fragments using a MiSeq 500-cycle sequencing kit version 2. Fastq files were generated and demultiplexed with the bcl2fastq v2.20 Conversion Software (Illumina).

A workflow was developed to perform four assemblies as follows, primarily to assess quality using different assembly strategies to find the best overall assembly. Initial assembly of the Oxford Nanopore data was carried out using Canu (NPL72), followed by polishing using Nanopolish (NPL73) and Pilon (utilizing the Illumina MiSeq reads—NPL74), and finally the genome was re-oriented using Circlator (NPL75). Another hybrid genome assembly was carried out using SPAdes (NPL76), followed by reorienting the genome using Circlator. A hybrid genome assembly was also carried out using Unicycler (NPL77). The final hybrid genome assembly was generated using Unicycler, with the Canu assembly above as the assembly backbone.

All assemblies were quality-assessed using BUSCO (NPL78) and QUAST (NPL79) and compared to a relevant reference genome using MUMmer. See NPL80. Assemblies were then followed by an annotation run using the tool Prokka (NPL81). After evaluation, the best overall assembly was determined using the best overall BUSCO scores in combination with overall assembly metrics.

### Assessment of the Molecular Weight of the Apoptotic Factor

Bacterial culture supernatants were prepared from cultures grown in Halomonas medium (8% NaCl, 0.75% casamino acids, 0.5% proteose peptone, 0.1% yeast extract, 0.3% sodium citrate, 2% magnesium sulfate heptahydrate, 0.05% potassium phosphate dibasic, 0.05% ammonium iron (II) sulfate hexahydrate) with shaking at 37° C. Bacterial cells were removed by centrifugation (17,000 x g, for 10 min at 4° C.) and filtration through 0.2 μm filters (Corning). Supernatants were size fractionated into high molecular weight (HMW) and low molecular weight (LMW) fractions by ultrafiltration with Ultracel-10K filters (Amicon), separated into aliquots and frozen at −20° C. In some experiments, bacterial culture supernatants were heat-treated (85° C., 15 min) before size fractionation. Equal volumes of supernatants were separated by 17.5% Tricine-sodium dodecyl sulfate-polyacrylamide gel electrophoresis (SDS-PAGE) and silver-stained using the Daiichi 2-D Silver Staining Kit (Daiichi, Tokyo, Japan).

### Cell Culture

The A549 and NHBE cells were cultured in DMEM supplemented with 10% fetal calf serum, 0.03% (w/v) L-glutamine, 100 IU/ml penicillin and 100 μg/ml streptomycin in a humidified, 5% CO₂ atmosphere at 37° C. We used A549 cell lines in most experiments because they have higher potential growth and mimic the phenotype of alveolar type II cells more than primary NHBE cells (NPL82, NPL83); and in addition, these primary cells usually easily change phenotype or become senescent after a short period of culture.

The bacterial culture supernatant (2 liters) was successively partitioned between n-hexane and water, and then ethyl acetate and water (2 L each, two times) (FIG. 13). The concentrated proteins were further concentrated under reduced pressure and then extracted with ethanol (2 liters each, two times). The ethanol-soluble portion (7.96 g) was fractionated by octadecyl silane gel flash column chromatography (5%; 10%, 20%, 50% methanol and methanol, 0.5 liter each) to obtain 42 fractions (fractions 1˜42). Fraction 42 (185.3 mg of proteins) was further separated by Sep-Pak (80% acetonitrile, methanol, and chloroform). Fraction 42-80% acetonitrile (75.6 mg of proteins) was separated by reverse-phase HPLC (C8, 80% methanol) to afford 22 fractions (fractions 42-80% acetonitrile-1˜22).

### Mass Spectrometry

Dried samples were suspended in 0.1% formic acid (FA) in 5% acetonitrile (ACN), and 2 μg of peptides were injected into a Thermo UltiMate 3000 UHPLC system. Reversed phase separation of sample peptides was accomplished using a 15 cm Acclaim PepMap 100 C18 column with mobile phases of 0.1% FA in water (A) and 0.1% FA in ACN (B). Peptides were eluted using a gradient of 2% B to 35% B over 60 minutes followed by 35% to 50% B over 5 minutes at a flow rate of 300 μl/min. The UHPLC system was coupled online to a Thermo Orbitrap Q-Exactive HFX (Biopharma Option) mass spectrometer operated in the data dependent mode. Precursor scans from 300 to 1,500 m/z (120,000 resolution) were followed by collision induced dissociation (CID) of the most abundant precursors over a maximum cycle time of 3 s (3e4 AGC, 35% NCE, 1.6 m/z isolation window, 60 s dynamic exclusion window).

The raw data were analyzed using Mascot 1.6 against a custom database containing the protein library of the Staphylococcus nepalensis CNDG genomic DNA, and the large and small plasmids encoded polypeptides (total of 3,541 protein sequences). No enzyme was specified. Peptide mass tolerance and fragment mass tolerances were set to 10 ppm and 0.1 Da, respectively. Variable modifications included oxidation of methionine residues (see mass spectrophotometry data in Supplementary Information).

### Apoptosis Assay

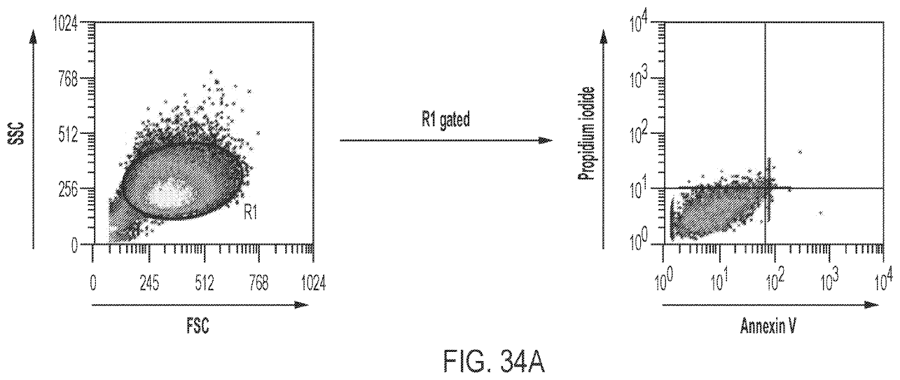

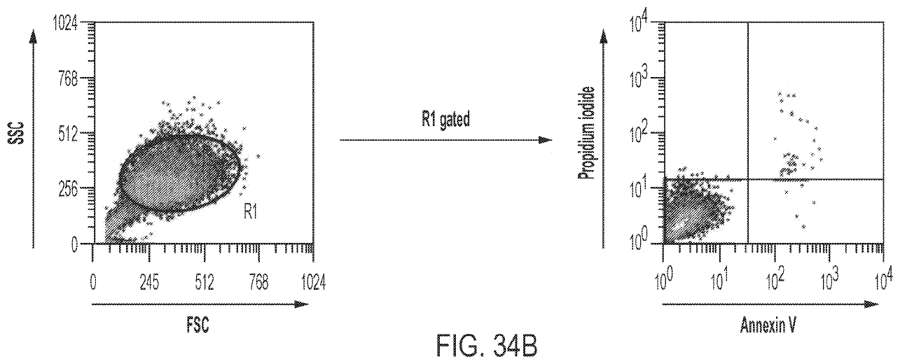

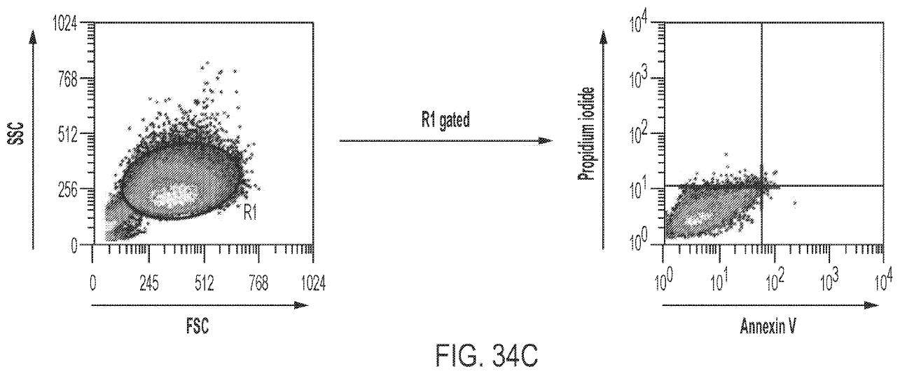

A549 and NBHE cells (4×10⁵ cells/well) were seeded into 12-well plates, cultured to sub-confluency, washed and then cultured in serum free medium containing 10% of each bacterial supernatant for 48 h. Non-inoculated hypersaline medium was used as control. The cells were analyzed for apoptosis by flow cytometry (FACScan, BD Biosciences, Oxford, UK) after staining with fluorescein-labelled annexin V and propidium iodide (FITC Annexin V Apoptosis Detection Kit with PI, Biolegend, San Diego, CA). Flow cytometry gating strategy used in the experiments is described in FIGS. 34A-34C. Under physiological conditions, phosphatidylcholine is exposed externally while phosphatidylserine (PS) is located on the inner surface of the lipid bilayer of cellular membranes. See NPL84. During apoptosis, PS is translocated from the cytoplasmic face of the plasma membrane to the cell surface. Id. Annexin V shows a strong affinity in binding to phosphatidylserine in a Ca²⁺-dependent manner and thus it is generally used as a probe for detecting apoptosis (see NPL85).

### Western Blotting

The cells for Western blot analysis were washed twice with ice-cold phosphate-buffered saline and then lysed in radioimmunoprecipitation assay (RIPA) buffer (10 mM Tris-Cl (pH 8.0), 1 mM EDTA, 1% Triton X-100, 0.1% sodium deoxycholate, 0.1% SDS, 140 mM NaCl, 1 mM phenylmethylsulfonyl fluoride) supplemented with protease/phosphatase inhibitors (1 mM orthovandate, 50 mM β-glycerophosphate, 10 mM sodium pyrophosphate, 5 μg/mL leupeptin, 2 μg/mL aprotinin, 5 mM sodium fluoride). The suspensions were centrifuged (17,000 x g, 10 min at 4° C.), and the protein content was determined using Pierce BCA protein assay kit (Thermo Fisher Scientific Incorporation, Waltham, MA). Equal amounts of cellular lysate protein were mixed with Laemmli sample buffer and separated by SDS-PAGE. Western blotting was then performed after electrophoretic transfer of proteins from sodium dodecyl sulfate-polyacrylamide gels to nitrocellulose membranes and using anti-phospho-Akt, anti-Akt, anti-cleaved caspase-3 or anti-β-actin antibody (Cell Signaling, Danvers, MA). See NPL67. The intensity of the bands was quantified by densitometry using the public domain NIH imageJ program (Wayne Rasband, NIH, Research Service Branch).

Immunohistochemistry

Staining of terminal deoxynucleotidyl transferase dUTP Nick-End Labeling (TUNEL) was performed at the Biopathology Institute Corporation (Kunisaki, Oita, Japan) by using Alexa Fluor 594 goat anti-rabbit IgG and slow-fade gold-antifade reagent with 4′,6-diamidino-2-phenylindole (DAPI) or by using ApopTag terminal deoxynucleotidyl transferase (Merck Millipore, Burlington, MA), anti-digoxigenin-peroxidase and 3,3′-diaminobenzidine. Quantification of apoptotic areas was performed using the WinROOF software (Mitani Corporation, Tokyo, Japan) and the values were averaged for each individual mouse.

### Evaluation of Gene Expression

We extracted total RNA from cells or lung tissue using Sepasol RNA-I Super G reagent (Nacalai Tesque Inc., Kyoto, Japan), synthesized cDNA from 2 μg of total RNA with oligo-dT primer and ReverTra Ace Reverse Transcriptase (Toyobo Life Science Department, Osaka, Japan) and then performed standard PCR using primers described in Table 5 below.

| | Sequence (5′→3′) | Tm | Reference | Location | Product size |
|---|---|---|---|---|---|
| Ctfr | | | | | |
| Sense | CACAGTCATCAACGGAATCGT | [symbol] | NM_021050 | 975-995 | 113 bp |
| Antisense | CATACCATATCTGTACGGCAGTG | [symbol] | | 1087-1065 | |
| [symbol] | | | | | |
| Sense | GCACCGACCATTAAGGACCTG | [symbol] | [symbol] | 64-84 | 118 bp |
| Antisense | GCGTGAACGCAATCCACAAC | [symbol] | | 181-182 | |
| [symbol] | | | | | |
| Sense | TACCTTGCGGAACTTCACCAG | [symbol] | NM_011325 | 603-623 | 136 bp |
| Antisense | CAAGCTAGGATTATGCGATCAGG | [symbol] | | 740-715 | |
| [symbol] | | | | | |
| Sense | TACTTCAGCTACCCCGTGAGT | [symbol] | NM_011324 | 403-423 | 153 bp |
| Antisense | AAAAAGCGTCTGTTCCGTGAT | [symbol] | | 555-535 | |
| TNFα | | | | | |
| Sense | ACGTGGAACTGGCAGAAGAG | [symbol] | [symbol] | 182-201 | 284 bp |
| Antisense | CTCCTCCACTTGGTGGTTTG | [symbol] | | [symbol] | |
| IFNγ | | | | | |
| Sense | GCTCTGACACAATGAACGCT | [symbol] | [symbol] | 99-118 | 229 bp |
| Antisense | AAAGAGATAATCTGGCTCTGC | [symbol] | | 327-307 | |
| [symbol] | | | | | |
| Sense | CACGGCATGGTTATTCCTTCA | [symbol] | [symbol] | 547-567 | 111 bp |
| Antisense | TCAGGACACGGTCAATGACAT | [symbol] | | 597-677 | |
| [symbol] | | | | | |
| Sense | CACAGTCATCAACGGAATCGT | [symbol] | [symbol] | 975-995 | 113 bp |
| Antisense | CATACCATATCTGTACGGCAGTG | [symbol] | | [symbol] | |
| [symbol] | | | | | |
| Sense | ACTCCACGTGGAAATCAACGG | [symbol] | [symbol] | [symbol] | 414 bp |
| Antisense | TAGTAGACGATGGGCAGTGG | [symbol] | | | |
| Vegf | | | | | |
| Sense | ATCTTCAAGCCGTCCTGTGTG | [symbol] | NM_009505 | 1232-1262 | 282 bp |
| Antisense | GCAGGAACATTTACACGTCTG | [symbol] | | 1513-1493 | |
| INOS | | | | | |
| Sense | TGGGAATGGAGACTGTCCCAG | [symbol] | NM_011577 | 1944-1954 | 306 bp |
| Antisense | GGGATCTGAATGTGATGTTTG | [symbol] | | 2249-2229 | |
| Mcp-1 | | | | | |
| Sense | ATGCAGGTCCCTGTCATGCTTC | [symbol] | [symbol] | 86-107 | 465 bp |
| Antisense | ACTAGTTCACTGTCACACTGGTC | [symbol] | | 533-511 | |
| [symbol] | | | | | |
| Sense | CAGGATGCAGAAGGAGATCAC | [symbol] | [symbol] | 1009-1029 | 354 bp |
| Antisense | TGTTGCTAGGCCAGGGCTAC | [symbol] | | 1372-1353 | |
| Fn1 | | | | | |
| Sense | TTCAAGTGTGATCCCCATGAAG | [symbol] | [symbol] | [symbol] | 154 bp |
| Antisense | CAGGTCTACGGCAGTTGTCA | [symbol] | | 7279-7260 | |
| Col1a | | | | | |
| Sense | TAAGGGTCCCCAATGGTGAGA | [symbol] | [symbol] | 107-127 | 203 bp |
| Antisense | GGGTCCCTCGACTCCTACAT | [symbol] | | 309-290 | |
| GAPDH | | | | | |
| Sense | TGGCCTTCCGTGTTCCTAC | [symbol] | NM_008084 | 686-704 | 176 bp |
| Antisense | GAGTTGCTGTTGAAGTCGCA | [symbol] | | [symbol] | |

[symbol]

[symbol]

indicates data missing or illegible when filed

PCR was performed with 26 to 35 cycles depending on the gene, denaturation at 94° C. for 30 s, annealing at 65° C. for 30 s, elongation at 72° C. for 1 min followed by a further extension at 72° C. for 5 min. See NPL67. The expression of mRNA was normalized against the glyceraldehyde 3-phosphate dehydrogenase (GAPDH) mRNA expression.

### Transmission Electron Microscopy of Apoptotic Cells

A549 cells (10×10⁴ cells/ml) were plated on a collagen-coated 8-well chamber slides (BD Bioscience, San Jose, CA) and cultured until semi-confluent. Cells were serum-starved for 6 h and stimulated with the pro-apoptotic peptide (5 μM) for 16 h. Cells were fixed with 2% fresh formaldehyde and 2.5% glutaraldehyde in 0.1 M sodium cacodylate buffer (pH 7.4) for 2 h at room temperature. After washing with 0.1 M cacodylate buffer (pH 7.4), they were postfixed with 1% OsO₄ in the same buffer for 2 h at 4° C. The samples were rinsed with distilled water, stained with 1% aqueous uranyl acetate for 2 h or overnight at room temperature, dehydrated with ethanol and propylene oxide, and embedded in epon (Epon 812 resin, Nakalai). After removal of the cells from the glass, ultra-thin sections (94 nm) were cut, stained with uranyl acetate and Reynolds's lead citrate, and viewed with a transmission electron microscope (JEM-1010, JEOL, Tokyo, Japan).

### Cell Cycle Analysis and Cell Viability Assay

We performed DNA content/cell cycle analysis by flow cytometry after culturing the cells for 48 h in the presence or absence of the bacterial supernatant fraction. Cell cycle distribution was evaluated after treating the cells with propidium iodide. Cell viability was performed using a commercial cell counting kit (Dojindo, Tokyo, Japan). The samples used in the assays were fractionated after gel filtration using a Sephadex G25 column.

Expression of S. nepalensis IsaA Transglycosylases

The genes encoding Staphylococcus nepalensis strain CNDG transglycosylase 351 and transglycosylase 531 were synthesized with E. coli optimized codons, amplified to add terminal A and cloned into the TA-cloning vector pGEM-T Easy (Promega, Madison, WI). The genes were then excised and cloned into a modified pET28a vector and transformed into E. coli BL21 DE3 cells and expressed and purified as 6-Histidine tagged (His-tag) proteins. See NPL86.

### Preparation of Antibody Against the Pro-Apoptotic Peptide

Protein A purified rabbit polyclonal antibody against the pro-apoptotic peptide (corisin) was developed by Eurofins Genomics (Tokyo, Japan) using the sequence NH2-C+IVMPESSGNPNAVNPAGYR-COOH (SEQ ID NO:1).

A band at the corresponding molecular weight for the target peptide can be observed in Western blotting of mouse lung tissue samples and culture supernatant of Staphylococcus nepalensis strain CNDG (FIGS. 22A and 22B).

### Corisin Detection and Measurement in Tissue and Body Fluids

The purified anti-corisin IgG antibody was used at 1/1000 dilution for Western blotting in lung tissue. We measured the concentration of corisin in body fluids using a competitive enzyme immune assay. Briefly, the purified corisin from transglycosylase 351 was coated on a 96-well plate at a final concentration of 2 μg/ml in phosphate-buffered saline at 4° C. overnight. After blocking and appropriate washing, the standards, samples and 5 ng/ml of anti-corisin were added to the wells and incubated at 4° C. overnight. The wells were then washed before adding horseradish peroxidase-conjugated goat anti-rabbit IgG (R&D System), as the secondary antibody, in a phosphate-buffered saline solution containing 5 μg/mL human IgG. After appropriate washing and incubation, substrate solution was added for color development and absorbance read at 450 nm. Values were extrapolated from a standard curve prepared using several concentrations of the peptide.

### Phylogenetic Analysis

The five transglycosylase polypeptides (CNDG_8p_00351, CNDG_8p_00513, CNDG_8p_00157, CNDG_8p_00159, and CNDG_8p_00845) were used to search the Genbank protein database (ncbi.nlm.nih.gov/protein/) to retrieve homologous proteins. The protein sequences were aligned with the MUltiple Sequence Comparison by Log-Expectation (MUSCLE) program and the alignment was used in generating a phylogenetic tree based on the neighbor joining method with bootstrap value of 1,000 replicates. All of these programs are available in Geneious Prime 2016 version (www.geneious.com).

More specifically, the phylogenetic tree shown in FIG. 29 was constructed by the Neighbor joining method. Bootstraps were performed with 1,000 replicates. The GenBank accession numbers in this tree are as follows: CLUSTER IsaA-1 ▪ [WP_112369066.1 (transglycosylase, S. arlettae), WP_061853755.1 (hypothetical protein, S. kloosii), WP_107393111.1 (transglycosylase, S. auricularis), WP_049409534.1 (hypothetical protein, S. pettenkoferi), WP_103371985.1 (transglycosylase, S. argensis), WP_046466985.1 (transglycosylase, S. pasteuri), COE35810.1 (transglycosylase, Streptococcus pneumoniae), WP_002467055.1 (hypothetical protein, S. warneri), WP_050969684.1 (transglycosylase, Streptococcus pneumoniae type N), WP_002449188.1 (hypothetical protein, S. hominis), WP_103166037.1 (transglycosylase, S. devriesei), WP_053024542.1 (transglycosylase, S. haemolyticus), WP_103328722.1 (transglycosylase, S. petrasii), WP_126565453.1 (transglycosylase, S. carnosus), WP_107511677.1 (transglycosylase, S. gallinarum), WP_069823097.1 (transglycosylase, S. succinus), WP_069833173.1 (transglycosylase, S. equorum), WP_057513458.1 (hypothetical protein, S. sp. NAM3COL9), WP_002506616.1 (hypothetical protein, S. sp. OJ82), WP_107552346.1 (transglycosylase, S. xylosus), WP_069827045.1 (transglycosylase, S. saprophyticus), WP_099091381.1 (transglycosylase, S. edaphicus), WP_073344326.1 (transglycosylase, S. cohnii), WP_119487699.1 (transglycosylase, S. nepalensis), CNDG_8p_00351 (putative transglycosylase IsaA-1, S. nepalensis)] CLUSTER IsaA-2 ▪ [SUK04795.1 SceA (S. aureus), WP_105995336.1 (hypothetical protein, S. agnetis), WP_105986821.1 (hypothetical protein, S. chromogenes), WP_009384111.1 (hypothetical protein, S. massiliensis), WP_126510217.1 (transglycosylase, S. epidermidis), WP_049407882.1 (hypothetical protein, S. pettenkoferi), WP_103371892.1 (hypothetical protein, S. argensis), WP_061853631.1 (hypothetical protein, S. kloosii), WP_107376802.1 (hypothetical protein, S. arlettae), WP_022791177.1 LysM peptidoglycan-binding domain-containing protein (Weissella halotolerans), WP_105993143.1 (hypothetical protein, S. simulans), WP_114602723.1 (hypothetical protein, S. sp. EZ-P03), WP_095089569.1 (hypothetical protein, S. stepanovicii), WP_017000663.1 (hypothetical protein, S. lentus), WP_119634381.1 (hypothetical protein, S. fleurettii), WP_126476519.1 (hypothetical protein, S. schleiferi), WP_107573021.1 (hypothetical protein, S. sciuri), WP_069822945.1 (hypothetical protein, S. succinus), WP_119484130.1 (hypothetical protein, S. gallinarum), WP_099090334.1 (hypothetical protein, S. edaphicus), WP_107558872.1 (hypothetical protein, S. xylosus), WP_069995535.1 (hypothetical protein, S. saprophyticus), WP_057513315.1 (hypothetical protein, S. sp. NAM3COL9), WP_069817445.1 (hypothetical protein, S. equorum), WP_107384366.1 (hypothetical protein, S. cohnii), CNDG_8p_00513 (putative transglycosylase IsaA-2, S. nepalensis), WP_096808504.1 (hypothetical protein, S. nepalensis)] CLUSTER SceD-1 ▪ [WP_101118359.1 (transglycosylase, S. succinus), WP_107530874.1 (transglycosylase, S. xylosus), WP_011302117.1 transglycosylase SceD 1 (S. saprophyticus), WP_105873943.1 (transglycosylase, S. cohnii), WP_107644182.1 (transglycosylase, S. nepalensis), CNDG_8p_00157 (putative transglycosylase SceD-1, S. nepalensis), WP_071564462.1 (transglycosylase, S. equorum)] CLUSTER SceD-2 ▪ [WP_070812670.1 (transglycosylase, S. sp. HMSC034G07), WP_119486153.1 (transglycosylase, S. gallinarum), WP_047504891.1 (transglycosylase, S. sp. ZWU0021), WP_057513650.1 (transglycosylase, S. sp. NAM3COL9), WP_096808177.1 (transglycosylase, S. nepalensis), CNDG_8p_00159 (putative transglycosylase SceD-2, S. nepalensis)] CLUSTER SceD-3 ▪ [WP_107564333.1 (transglycosylase, S. succinus), WP_115347167.1 (transglycosylase, S. saprophyticus), WP_107557548.1 (transglycosylase, S. xylosus), WP_099091190.1 (transglycosylase, S. edaphicus), WP_064263215.1 (transglycosylase, S. cohnii), CNDG_8p_00161 (putative transglycosylase SceD-3, S. nepalensis), WP_107644349.1 (transglycosylase, S. nepalensis)] CLUSTER SceD-4 ▪ [WP_119569949.1 (transglycosylase, S. succinus), WP_107385877.1 (transglycosylase, S. cohnii), CNDG_8p_00845 (putative transglycosylase SceD-4, S. nepalensis), WP_096808795.1 (transglycosylase, S. nepalensis)]. WP_050969685.1 (transglycosylase, Streptococcus pneumoniae type N), YP_501340.1 (transglycosylase, S. aureus subsp. aureus NCTC 8325), WP_046206716.1 (transglycosylase, S. cohnii subs. cohnii)

### Statistical Analysis

Data are described as the mean ±standard deviation of the means (S.D.) unless otherwise specified. The statistical difference between two variables was assessed by Mann-Whitney U test and the difference between three or more variables by analysis of variance using Tukey's test for post-hoc analysis. P value <0.05 was considered statistically significant. We performed the statistical analysis using GraphPad Prism vs 7 (GraphPad Software, Inc., San Diego, CA).

Additional embodiments of the present disclosure include, but are not limited to:
1. A method for evaluating fibrosis comprising detecting corisin as a target substance.
2. The method according to the above-mentioned Embodiment 1, wherein the 19 amino acid sequence (IVMPESSGNPNAVNPAGYR—SEQ ID NO: 1) in corisin is detected.
3. The method according to the above-mentioned Embodiment 1 or 2, wherein the fibrosis is selected from the group consisting of idiopathic pulmonary fibrosis (IPF), liver cirrhosis, kidney fibrosis, cystic fibrosis, myelofibrosis, and mammary fibrosis.
4. An antibody that binds to corisin and that prevents and/or treats fibrosis.
5. The antibody according to the above-mentioned Embodiment 5, wherein the antibody recognizes the 19 amino acid sequence (IVMPESSGNPNAVNPAGYR—SEQ ID NO: 1).
6. The antibody according to the above-mentioned Embodiment 4 or 5, wherein the antibody is a polyclonal antibody.
7. A method for identifying a corisin receptor protein, comprising searching for a corisin-binding protein that exists on the surface of epithelial cells.
8. A method for identifying a corisin receptor protein, comprising searching for a 19 amino acid sequence (IVMPESSGNPNAVNPAGYR—SEQ ID NO: 1) of a binding protein that exists on the surface of epithelial cells.

### Non-Patent Literature (“NPL”) References Mentioned in the Description Above

NPL1. Ley B, Collard H R, King T E, Jr. Clinical course and prediction of survival in idiopathic pulmonary fibrosis. Am J_Respir Crit Care Med 183, 431-440 (2011). doi: 10.1164/rccm.201006-0894CI
NPL2. Richeldi L, Collard H R, Jones M G. Idiopathic pulmonary fibrosis. Lancet 389, 1941-1952 (2017). doi: 10.1016/S0140-6736(17)30866-8
NPL3. du Bois R M. An earlier and more confident diagnosis of idiopathic pulmonary fibrosis. Eur Respir Rev 21, 141-146 (2012). doi: 10.1183/09059180.00000812
NPL4. King T E, Jr., Pardo A, Selman M. Idiopathic pulmonary fibrosis. Lancet 378, 1949-1961 (2011). doi: 10.1016/S0140-6736(11)60052-4
NPL5. King T E, Jr., Noble P W, Bradford W Z. Treatments for idiopathic pulmonary fibrosis. N Engl J Med 371, 783-784 (2014). doi: 10.1056/NEJMc1407776
NPL6. Molyneaux P L, et al. The role of bacteria in the pathogenesis and progression of idiopathic pulmonary fibrosis. Am J Respir Crit Care Med 190, 906-913 (2014). doi: 10.1164/rccm.201403-0541OC
NPL7. Han M K, et al. Lung microbiome and disease progression in idiopathic pulmonary fibrosis: an analysis of the COMET study. Lancet Respir Med 2, 548-556 (2014). doi: 10.1016/S2213-2600(14)70069-4
NPL8. D'Alessandro-Gabazza C N, et al. Identification of Halophilic Microbes in Lung Fibrotic Tissue by Oligotyping. Front Microbiol 9, 1892 (2018). doi: 10.3389/fmicb.2018.01892
NPL9. O'Dwyer D N, et al. Lung Microbiota Contribute to Pulmonary Inflammation 20 and Disease Progression in Pulmonary Fibrosis. Am J Respir Crit Care Med 199, 1127-1138 (2019). doi: 10.1164/rccm.201809-1650OC
NPL10. Caja L, et al. TGF-beta and the Tissue Microenvironment: Relevance in Fibrosis and Cancer. Int J Mol Sci 19, e1294 (2018). doi: 10.3390/ijms19051294
NPL11. D'Alessandro-Gabazza C N, et al. Development and preclinical efficacy of novel transforming growth factor-betal short interfering RNAs for pulmonary fibrosis. Am J Respir Cell Mot Biol 46, 397-406 (2012). doi: 10.1165/rcmb.2011-0158OC
NPL12. Zhao X, Kwan J Y Y, Yip K, Liu P P, Liu F F. Targeting metabolic dysregulation for fibrosis therapy. Nat Rev Drug Discov 19, 57-75 (2020). doi: 10.1038/s41573-019-0040-5
NPL13. Haruyama N, Cho A, Kulkarni A B. Overview: engineering transgenic constructs and mice. Curr Protoc Cell Biol Chapter 19, Unit 19 10 (2009). doi: 10.1002/0471143030.cb1910s42
NPL14. Schatz V, et al. Elementary immunology: Na(+) as a regulator of immunity. Pediatr Nephrol 32, 201-210 (2017). doi: 10.1007/s00467-016-3349-x
NPL15. Konishi K, et al. Gene expression profiles of acute exacerbations of idiopathic pulmonary fibrosis. Am J Respir Crit Care Med 180, 167-175 (2009). doi: 10.1164/rccm.200810-1596OC
NPL16. Plataki M, Koutsopoulos A V, Darivianaki K, Delides G, Siafakas N M, Bouros D. Expression of apoptotic and antiapoptotic markers in epithelial cells in idiopathic pulmonary fibrosis. Chest 127, 266-274 (2005). doi: 10.1378/chest.127.1.266
NPL17. Fernandez I E, Eickelberg O. The impact of TGF-beta on lung fibrosis: from targeting to biomarkers. Proc Am Thorac Soc 9, 111-116 (2012). doi: 10.1513/pats.201203-023AW
NPL18. Rockey D C, Bell P D, Hill J A. Fibrosis—A Common Pathway to Organ Injury and Failure. N Engl J Med 373, 96 (2015). doi: 10.1056/NEJMc1504848
NPL19. Aschner Y, Downey G P. Transforming Growth Factor-beta: Master Regulator of the Respiratory System in Health and Disease. Am J Respir Cell Mol Biol 54, 647-655 (2016). doi: 10.1165/rcmb.2015-0391TR
NPL20. Azadeh N, Limper A H, Carmona E M, Ryu J H. The Role of Infection in Interstitial Lung Diseases: A Review. Chest 152, 842-852 (2017). doi: 10.1016/j.chest.2017.03.033
NPL21. Thomas B J, Kan O K, Loveland K L, Elias J A, Bardin P G. In the Shadow of Fibrosis: Innate Immune Suppression Mediated by Transforming Growth Factor-beta. Am J Respir Cell Mol Biol 55, 759-766 (2016). doi: 10.1165/rcmb.2016-0248PS
NPL22. Smith J J, Travis S M, Greenberg E P, Welsh M J. Cystic fibrosis airway epithelia fail to kill bacteria because of abnormal airway surface fluid. Cell 85, 229-236 (1996). doi: [symbol]
NPL23. Willebrand R, Kleinewietfeld M. The role of salt for immune cell function and disease. Immunology 154, 346-353 (2018). doi: 10.1111/imm.12915
NPL24. Zabner J, et al. The osmolyte xylitol reduces the salt concentration of airway surface liquid and may enhance bacterial killing. Proc Natl Acad Sci USA 97, 11614-11619 (2000). doi: 10.1073/pnas.97.21.11614
NPL25. Frank J A, Matthay M A. TGF-beta and lung fluid balance in ARDS. Proc Natl Acad Sci USA 111, 885-886 (2014). doi: 10.1073/pnas.1322478111
NPL26. Peters D M, et al. TGF-beta directs trafficking of the epithelial sodium channel ENaC which has implications for ion and fluid transport in acute lung injury. Proc Natl Acad Sci USA 111, E374-383 (2014). doi: 10.1073/pnas.1306798111
NPL27. Frank J, et al. Transforming growth factor-betal decreases expression of the epithelial sodium channel alphaENaC and alveolar epithelial vectorial sodium and fluid transport via an ERK1/2-dependent mechanism. J Biol Chem 278, 43939-43950 (2003). doi: 10.1074/jbc.M304882200
NPL28. Lutful Kabir F, et al. MicroRNA-145 Antagonism Reverses TGF-beta Inhibition of F508del CFTR Correction in Airway Epithelia. Am J Respir Crit Care Med 197, 632-643 (2018). doi: 10.1164/rccm.201704-0732OC
NPL29. Sun H, et al. Tgf-beta downregulation of distinct chloride channels in cystic fibrosis-affected epithelia. PLoS One 9, e106842 (2014). doi: 10.1371/journal.pone.0106842
NPL30. Xu Y, et al. Single-cell RNA sequencing identifies diverse roles of epithelial cells in idiopathic pulmonary fibrosis. JCI Insight 1, e90558 (2016). doi: 10.1172/jci.insight.90558
NPL31. Burgstaller G, Oehrle B, Gerckens M, White E S, Schiller H B, Eickelberg O. The instructive extracellular matrix of the lung: basic composition and alterations in chronic lung disease. Eur Respir J 50, pii: 1601805 (2017). doi: 10.1183/13993003.01805-2016
NPL32. Fischereder M, et al. Sodium storage in human tissues is mediated by glycosaminoglycan expression. Am J Physiol Renal Physiol 313, F319-F325 (2017). doi: 10.1152/ajprenal.00703.2016
NPL33. Lu J, Auduong L, White E S, Yue X. Up-regulation of heparan sulfate 6-0-sulfation in idiopathic pulmonary fibrosis. Am J Respir Cell Mol Biol 50, 106-114 (2014). doi: 10.1165/rcmb.2013-0204OC
NPL34. Titze J, et al. Osmotically inactive skin Na+ storage in rats. Am J Physiol Renal Physiol 285, F1108-1117 (2003). doi: 10.1152/ajprenal.00200.2003
NPL35. Westergren-Thorsson G, et al. Increased deposition of glycosaminoglycans and altered structure of heparan sulfate in idiopathic pulmonary fibrosis. Int J Biochem Cell Biol 83, 27-38 (2017). doi: 10.1016/j.biocel.2016.12.005
NPL36. Collard H R, et al. Acute Exacerbation of Idiopathic Pulmonary Fibrosis. An International Working Group Report. Am J Respir Crit Care Med 194, 265-275 (2016). doi: 10.1164/rccm.201604-0801CI
NPL37. Collard H R, Yow E, Richeldi L, Anstrom K J, Glazer C, investigators IP. Suspected acute exacerbation of idiopathic pulmonary fibrosis as an outcome measure in clinical trials. Respir Res 14, 73 (2013). doi: 10.1186/1465-9921-14-73
NPL38. Jeon K, et al. Prognostic factors and causes of death in Korean patients with idiopathic pulmonary fibrosis. Respir Med 100, 451-457 (2006). doi: 10.1016/j.rmed.2005.06.013
NPL39. Kondoh Y, et al. Risk factors of acute exacerbation of idiopathic pulmonary fibrosis. Sarcoidosis Vasc Diffuse Lung Dis 27, 103-110 (2010). doi:
NPL40. Natsuizaka M, et al. Epidemiologic survey of Japanese patients with idiopathic pulmonary fibrosis and investigation of ethnic differences. Am J Respir Crit Care Med 190, 773-779 (2014). doi: 10.1164/rccm.201403-0566OC
NPL41. Song J W, Hong S B, Lim C M, Koh Y, Kim D S. Acute exacerbation of idiopathic pulmonary fibrosis: incidence, risk factors and outcome. Eur Respir J 37, 356-363(2011). doi: 10.1183/09031936.00159709
NPL42. Huang Y, et al. Microbes Are Associated with Host Innate Immune Response in Idiopathic Pulmonary Fibrosis. Am J Respir Crit Care Med 196, 208-219 (2017). doi: 10.1164/rccm.201607-1525OC
NPL43. Kawamura K, Ichikado K, Yasuda Y, Anan K, Suga M. Azithromycin for idiopathic acute exacerbation of idiopathic pulmonary fibrosis: a retrospective single-center study. BMC Pulm Med 17, 94 (2017). doi: 10.1186/s12890-017-0437-z
NPL44. Macaluso C, et al. The potential impact of azithromycin in idiopathic pulmonary fibrosis. Eur Respir J53, pii:1800628 (2019). doi: 10.1183/13993003.00628-2018
NPL45. Molyneaux P L, et al. Changes in the respiratory microbiome during acute exacerbations of idiopathic pulmonary fibrosis. Respir Res 18, 29 (2017). doi: 10.1186/s12931-017-0511-3
NPL46. Shulgina L, et al. Treating idiopathic pulmonary fibrosis with the addition of co-trimoxazole: a randomised controlled trial. Thorax 68, 155-162 (2013). doi: 10.1136/thoraxjnl-2012-202403
NPL47. Varney V A, Parnell H M, Salisbury D T, Ratnatheepan S, Tayar R B. A double blind randomised placebo controlled pilot study of oral co-trimoxazole in advanced fibrotic lung disease. Pulm Pharmacol Ther 21, 178-187 (2008). doi: 10.1016/j.pupt.2007.02.001
NPL48. Kitsios G D, et al. Microbiome in lung explants of idiopathic pulmonary fibrosis: a case-control study in patients with end-stage fibrosis. Thorax 73, 481-484 (2018). doi: 10.1136/thoraxjn1-2017-210537
NPL49. Knippenberg S, et al. Streptococcus pneumoniae triggers progression of pulmonary fibrosis through pneumolysin. Thorax 70, 636-646 (2015). doi: 10.1136/thoraxjnl-2014-206420
NPL50. Takahashi Y, et al. Impaired diversity of the lung microbiome predicts progression of idiopathic pulmonary fibrosis. Respir Res 19, 34 (2018). doi: 10.1186/s12931-018-0736-9
NPL51. Tong X, et al. Alterations to the Lung Microbiome in Idiopathic Pulmonary Fibrosis Patients. Front Cell Infect Microbiol 9, 149 (2019). doi: 10.3389/fcimb.2019.00149
NPL52. Yang D, et al. Dysregulated Lung Commensal Bacteria Drive Interleukin-17B Production to Promote Pulmonary Fibrosis through Their Outer Membrane Vesicles. Immunity 50, 692-706 e697 (2019). doi: 10.1016/j.immuni.2019.02.001
NPL53. Stapleton M R, et al. Characterization of IsaA and SceD, two putative lytic transglycosylases of Staphylococcus aureus. J Bacteriol 189, 7316-7325 (2007). doi: 10.1128/JB.00734-07
NPL54. Ellson C D, Dunmore R, Hogaboam C M, Sleeman M A, Murray L A. Danger-associated molecular patterns and danger signals in idiopathic pulmonary fibrosis. Am Respir Cell Mot Biol 51, 163-168 (2014). doi: 10.1165/rcmb.2013-0366TR
NPL55. Dik D A, Marous D R, Fisher J F, Mobashery S. Lytic transglycosylases: concinnity in concision of the bacterial cell wall. Crit Rev Biochem Mol Biol 52, 503-542 (2017). doi: 10.1080/10409238.2017.1337705
NPL56. Heidrich C, Ursinus A, Berger J, Schwarz H, Holtje J V. Effects of multiple deletions of murein hydrolases on viability, septum cleavage, and sensitivity to large toxic molecules in Escherichia coli. J Bacteriol 184, 6093-6099 (2002). doi: 10.1128/jb.184.22.6093-6099.2002
NPL57. Scheurwater E M, Clarke A J. The C-terminal domain of Escherichia coli YfhD functions as a lytic transglycosylase. J Biol Chem 283, 8363-8373 (2008). doi: 10.1074/jbc.M710135200
NPL58. Invernizzi R, Molyneaux P L. The contribution of infection and the respiratory microbiome in acute exacerbations of idiopathic pulmonary fibrosis. Eur Respir Rev 28, pii:190045 (2019). doi: 10.1183/16000617.0045-2019
NPL59. McShane P J, Glassroth J. Pulmonary Disease Due to Nontuberculous Mycobacteria: Current State and New Insights. Chest 148, 1517-1527 (2015). doi: 10.1378/chest.15-0458
NPL60. Moffatt M F, Cookson W O. The lung microbiome in health and disease. Clin Med (Lond) 17, 525-529 (2017). doi: 10.7861/clinmedicine.17-6-525
NPL61. Oda K, et al. Respiratory comorbidities and risk of mortality in hospitalized patients with idiopathic pulmonary fibrosis. Respir Investig 56, 64-71 (2018). doi: 10.1016/j.resinv.2017.09.006
NPL62. Sauler M, Bazan I S, Lee P J. Cell Death in the Lung: The Apoptosis-Necroptosis Axis. Annu Rev Physiol 81, 375-402 (2019). doi: 10.1146/annurev-physiol-020518-114320
NPL63. Moskowitz S M, Wiener-Kronish J P. Mechanisms of bacterial virulence in pulmonary infections. Curr Opin Crit Care 16, 8-12 (2010). doi: 10.1097/MCC.0b013e3283354710
NPL64. Marsland B J, Gollwitzer E S. Host-microorganism interactions in lung diseases. Nat Rev Immunol 14, 827-835 (2014). doi: 10.1038/nri3769
NPL65. Meyer K C, et al. An official American Thoracic Society clinical practice guideline: the clinical utility of bronchoalveolar lavage cellular analysis in interstitial lung disease. Am J Respir Crit Care Med 185, 1004-1014 (2012). doi: 10.1164/rccm.201202-0320ST
NPL66. Travis W D, et al. An official American Thoracic Society/European Respiratory Society statement: Update of the international multidisciplinary classification of the idiopathic interstitial pneumonias. Am J Respir Crit Care Med 188, 733-748 (2013). doi: 10.1164/rccm.201308-1483ST
NPL67. Fujiwara K, et al. Inhibition of Cell Apoptosis and Amelioration of Pulmonary Fibrosis by Thrombomodulin. Am J Pathol 187, 2312-2322 (2017). doi: 10.1016/j.ajpath.2017.06.013
NPL68. Yasui H, et al. Intratracheal administration of activated protein C inhibits bleomycin-induced lung fibrosis in the mouse. Am J Respir Crit Care Med 163, 1660-1668 (2001). doi: 10.1164/ajrccm.163.7.9911068
NPL69. Kleinewietfeld M, et al. Sodium chloride drives autoimmune disease by the induction of pathogenic TH17 cells. Nature 496, 518-522 (2013). doi: 10.1038/nature11868
NPL70. Machnik A, et al. Macrophages regulate salt-dependent volume and blood pressure by a vascular endothelial growth factor-C-dependent buffering mechanism. Nat Med 15, 545-552 (2009). doi: 10.1038/nm.1960
NPL71. Cann I K, Stroot P G, Mackie K R, White B A, Mackie R I. Characterization of two novel saccharolytic, anaerobic thermophiles, Thermoanaerobacterium polysaccharolyticum sp. nov. and Thermoanaerobacterium zeae sp. nov., and emendation of the genus Thermoanaerobacterium. Int J Syst Evol Microbiol 51, 293-302 (2001). doi: 10.1099/00207713-51-2-293
NPL72. Koren S, Walenz B P, Berlin K, Miller J R, Bergman N H, Phillippy A M. Canu: scalable and accurate long-read assembly via adaptive k-mer weighting and repeat separation. Genome Res 27, 722-736 (2017). doi: 10.1101/gr.215087.116
NPL73. Loman N J, Quick J, Simpson J T. A complete bacterial genome assembled de novo using only nanopore sequencing data. Nat Methods 12, 733-735 (2015). doi: 10.1038/nmeth.3444
NPL74. Walker B J, et al. Pilon: an integrated tool for comprehensive microbial variant detection and genome assembly improvement. PLoS One 9, e112963 (2014). doi: 10.1371/journal.pone.0112963
NPL75. Hunt M, Silva N D, Otto T D, Parkhill J, Keane J A, Harris S R. Circlator: automated circularization of genome assemblies using long sequencing reads. Genome Biol 16, 294 (2015). doi: 10.1186/s13059-015-0849-0
NPL76. Bankevich A, et al. SPAdes: a new genome assembly algorithm and its applications to single-cell sequencing. J Comput Biol 19, 455-477 (2012). doi: 10.1089/cmb.2012.0021
NPL77. Wick R R, Judd L M, Gorrie C L, Holt K E. Unicycler: Resolving bacterial genome assemblies from short and long sequencing reads. PLoS Comput Biol 13, e1005595 (2017). doi: 10.1371/journal.pcbi.1005595
NPL78. Waterhouse R M, et al. BUSCO applications from quality assessments to gene prediction and phylogenomics. Mol Biol Evol 35, 543-548 (2017). doi: 10.1093/molbev/msx319
NPL79. Gurevich A, Saveliev V, Vyahhi N, Tesler G. QUAST: quality assessment tool for genome assemblies. Bioinformatics 29, 1072-1075 (2013). doi: 10.1093/bioinformatics/btt086
NPL80. Delcher A L, Salzberg S L, Phillippy A M. Using MUMmer to identify similar regions in large sequence sets. Curr Protoc Bioinformatics Chapter 10, Unit 10 13 (2003). doi: 10.1002/0471250953.bi1003s00
NPL81. Seemann T. Prokka: rapid prokaryotic genome annotation. Bioinformatics 30, 2068-2069 (2014). doi: 10.1093/bioinformatics/btu153
NPL82. Foster K A, Oster C G, Mayer M M, Avery M L, Audus K L. Characterization of the A549 cell line as a type II pulmonary epithelial cell model for drug metabolism. Exp Cell Res 243, 359-366 (1998). doi: 10.1006/excr.1998.4172
NPL83. Nardone L L, Andrews S B. Cell line A549 as a model of the type II pneumocyte. Phospholipid biosynthesis from native and organometallic precursors. Biochim Biophys Acta 573, 276-295 (1979). doi: 10.1016/0005-2760(79)90061-4
NPL84. Fadok V A, Voelker D R, Campbell P A, Cohen J J, Bratton D L, Henson P M. Exposure of phosphatidylserine on the surface of apoptotic lymphocytes triggers specific recognition and removal by macrophages. J Immunol 148, 2207-2216 (1992).
NPL85. Koopman G, Reutelingsperger C P, Kuijten G A, Keehnen R M, Pals S T, van Oers M H. Annexin V for flow cytometric detection of phosphatidylserine expression on B cells undergoing apoptosis. Blood 84, 1415-1420 (1994).
NPL86. Zhang M, et al. Xylan utilization in human gut commensal bacteria is orchestrated by unique modular organization of polysaccharide-degrading enzymes. PNAS 111 (35) E3708-E3717 (2014). doi: 10.1073/pnas.1406156111

1-8. (canceled)

9. An antibody that binds to corisin.

10. The antibody according to claim 9, wherein the antibody recognizes an amino acid sequence selected from the group consisting of SEQ ID NO: 1, SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, and SEQ ID No: 13.

11. The antibody according to claim 9, wherein the antibody is a polyclonal antibody.

12. The antibody according to claim 9, wherein the antibody is for use in preventing, ameliorating and/or treating fibrosis in a patient subject having, or suspected of having or developing, fibrosis.

13. The antibody according to claim 12, wherein the fibrosis is selected from the group consisting of idiopathic pulmonary fibrosis (IPF), liver cirrhosis, kidney fibrosis, cystic fibrosis, myelofibrosis, and mammary fibrosis.

14. (canceled)

15. The antibody according to claim 9, wherein the antibody is a neutralizing antibody.

16. A method of treating fibrosis in a patient in need thereof comprising administering a therapeutically effective amount of the antibody of claim 9 to the patient.

17. (canceled)

18. The method according to claim 16, wherein the antibody is administered intraperitoneally or by intratracheal instillation or by inhalation.

19. The method according to claim 16, wherein administration of the antibody reduces the severity of the fibrosis in the subject.

20. A method for use in evaluating a subject having, or suspected of having or developing, fibrosis, the method comprising:
receiving an in vitro biological sample collected from the subject; and
detecting an amount of corisin that is present in the in vitro biological sample.

21. The method according to claim 20, further comprising:
comparing the detected amount of corisin in the in vitro biological sample to one or more predetermined thresholds.

22. (canceled)

23. The method according to claim 20, wherein the in vitro biological sample is selected from the group consisting of sputum, bronchial secretion, pleural effusion, bronchoalveolar lavage fluid (BALF), blood, and tissue collected from the bronchus or the lung.

24. (canceled)

25. The method according to claim 20, wherein the corisin has an amino acid sequence selected from the group consisting of SEQ ID NO: 1, SEQ ID No: 4, SEQ ID No: 5, SEQ ID No: 6, SEQ ID NO: 7, SEQ ID No: 8, SEQ ID No: 9, SEQ ID No: 10, SEQ ID NO: 11, SEQ ID No: 12, and SEQ ID No: 13.

26. The method according to claim 20, wherein the fibrosis is selected from the group consisting of idiopathic pulmonary fibrosis (IPF), liver cirrhosis, kidney fibrosis, cystic fibrosis, myelofibrosis, and mammary fibrosis.

27. (canceled)

28. The method according to claim 20, wherein the corisin is detected by a method selected from the group consisting of mass spectrometry, Western blotting, and enzyme-linked immunosorbent assay (ELISA).

29. The method according to claim 20, wherein corisin is detected by binding to antibody binding.

30-35. (canceled)

36. A method for identifying a corisin receptor protein, comprising searching for a corisin-binding protein present on a surface of an epithelial cell.

37-40. (canceled)

41. A pharmaceutical composition for use in the treatment of fibrosis in a patient, the pharmaceutical composition comprising:
the antibody of claim 9, and
at least one pharmaceutically acceptable additive, salt or excipient.

42. A method of treating fibrosis in a patient in need thereof, comprising administering a therapeutically effective amount of the pharmaceutical composition of claim 41 to the patient.

43-45. (canceled)

46. The method according to claim 20, further comprising administering a corisin inhibitor to the subject.
